SEQUENCE LISTING (31 sequences)

SEQ ID NO: 1 - protein, 775 aa, Pyrococcus furiosus
  source 1..775: note = Pyrococcus furiosus (pfu) polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 2 - protein, 775 aa, synthetic construct
  source 1..775: note = Pfu-RTX
MILDVDYITEEGKPVIRLFKKENGKFKIEHDRTFRPYLYALLRDDSKIEEVKKITGERHGKIVRIVDVEKVEKKFLGKPITVWKLYLEHPQDVPTIMEKVREHPAVVDIFEYDIPFAIRYLIDKGLIPMEGEEELKLLAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLHESHTGGFIKEPEKGLWENIVYLDFRALYPSIIITHNVSPDTLNLEGCKNYDIAPQVGHKFCKDIPGFIPSLLGHLLEERQKIKTRMKETQDPIEKILLDYRQKAIKLLANSLYGYYGYAKARWYCKECAESVIAWGRKYLELVWKELEEKFGFKVLYIDTDGLYATIPGGESEEIKKKALEFVKYINSKLPGLLELEYEGFYKRGLFVTKKRYAVIDEEGKVITRGLEIVRRDWSEIAKETQARVLETILKHGDVEEAVRIVKEVIQKLANYEIPPEKLAIYKQITRPLHEYKAIGPHVAVAKKLAAKGVKIKPGMVIGYIVLRGDGPIVNRAILAEEYDPKKHKYDAEYYIEKQVLPAVLRILEGFGYRKEDLRYQKTRQVGLTSRLNIKKS

SEQ ID NO: 3 - protein, 775 aa, synthetic construct
  source 1..775: note = Pfu-RTXxo-1
MILDVDYITEEGKPVIRLFKKENGKFKIEHDRTFRPYLYALLRDDSKIEEVKKITGERHGKIVRIVDVEKVEKKFLGKPITVWKLYLEHPQDVPTIMEKVREHPAVVDIFEYDIPFAIRYLIDKGLIPMEGEEELKLLAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLHESHTGGFIKEPEKGLWENIVYLDFRALYPSIIITHNVSPDTLNLEGCKNYDIAPQVGHKFCKDIPGFIPSLLGHLLEERQKIKTRMKETQDPIEKILLDYRQKAIKLLANSLYGYYGYAKARWYCKECAESVIAWGRKYLELVWKELEEKFGFKVLYIDTDGLYATIPGGESEEIKKKALEFVKYINSKLPGLLELEYEGFYKRGLFVTKKRYAVIDEEGKVITRGLEIVRRDWSEIAKETQARVLETILKHGDVEEAVRIVKEVIQKLANYEIPPEKLAIYKQITRPLHEYKAIGPHVAVAKKLAAKGVKIKPGMVIGYIVLRGDGPIVNRAILAEEYDPKKHKYDAEYYIEKQVLPAVLRILEGFGYRKEDLRYQKTRQVGLTSRLNIKKS

SEQ ID NO: 4 - protein, 775 aa, synthetic construct
  source 1..775: note = Pfu-RTXxo-2
MVLDVDYITEEGKPVIRLFKKENGKFKIEHDRTFRPYLYALLRDDSKIEEVKKITGERHGKIVRIVDVEKVEKKFLGKPITVWKLYLEHPQDVPTIMEKVREHPAVVDIFEYDIPFAIRYLIDKGLIPMEGEEELKLLAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLHESHTGGFIKEPEKGLWENIVYLDFRALYPSIIITHNVSPDTLNLEGCKNYDIAPQVGHKFCKDIPGFIPSLLGHLLEERQKIKTRMKETQDPIEKILLDYRQKAIKLLANSLYGYYGYAKARWYCKECAESVIAWGRKYLELVWKELEEKFGFKVLYIDTDGLYATIPGGESEEIKKKALEFVKYINSKLPGLLELEYEGFYKRGLFVTKKRYAVIDEEGKVITRGLEIVRRDWSEIAKETQARVLETILKHGDVEEAVRIVKEVIQKLANYEIPPEKLAIYKQITRPLHEYKAIGPHVAVAKKLAAKGVKIKPGMVIGYIVLRGDGPIVNRAILAEEYDPKKHKYDAEYYIEKQVLPAVLRILEGFGYRKEDLRYQKTRQVGLTSRLNIKKS

SEQ ID NO: 5 - protein, 775 aa, synthetic construct
  source 1..775: note = Pfu-RTXxo-3
MVLDVDYITEEGKPVIRLFKKENGKFKIEHDRTFRPYLYALLRDDSKIEEVKKITGERHGKIVRIVDVEKVEKKFLGKPITVWKLYLEHPQDQPTIMEKVREHPAVVDIFEYDIPFAIRYLIDKGLIPMEGEEELKLLAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLHESHTGGFIKEPEKGLWENIVYLDFRALYPSIIITHNVSPDTLNLEGCKNYDIAPQVGHKFCKDIPGFIPSLLGHLLEERQKIKTRMKETQDPIEKILLDYRQKLIKLLANSLYGYYGYAKARWYCKECAESVIAWGRKYLELVWKELEEKFGFKVLYIDTDGLYATIPGGESEEIKKKALEFVKYINSKLPGLLELEYEGFYKRGLFVTKKRYAVIDEEGKVITRGLEIVRRDWSEIAKETQARVLETILKHGDVEEAVRIVKEVIQKLANYEIPPEKLAIYKQITRPLHEYKAIGPHVAVAKKLAAKGVKIKPGMVIGYIVLRGDGPIVNRAILAEEYDPKKHKYDAEYYIEKQVLPAVLRILEGFGYRKEDLRYQKTRQVGLTSRLNIKKS

SEQ ID NO: 6 - protein, 774 aa, Thermococcus kodakarensis
  source 1..774: note = Thermococus kodakarensis (KOD1) polymerase
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 7 - protein, 774 aa, synthetic construct
  source 1..774: note = KOD-RTX
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGT

SEQ ID NO: 8 - protein, 774 aa, unidentified
  source 1..774: note = Wild-Type KodPol
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYEEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYERGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 9 - protein, 774 aa, synthetic construct
  source 1..774: note = KOD-RTX
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYEEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYERGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGT

SEQ ID NO: 10 - protein, 773 aa, Thermococcus gorgonarius
  source 1..773: note = Thermococcus gorgonarius polymerase (Tgo polymerase)
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 11 - protein, 781 aa, synthetic construct
  source 1..781: note = TgoRTxo
MVLDTDYITEDGKPVIRIFKKENGEFKIDYDRNFEPYLYALLKDDSAIEDVKKITAERHGTTVRVVRAEKVKKKFLGRPIEVWKLYFTHPQDVPAIMDKIKEHPAVVDIYEYDIPFAIRYLIDKGLIPMEGDEELKLLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRHESHAGGYIKEPERGLWENIVYLDFRSLYPSIIITHNVSPDTLNREGCEEYDVAPQVGHKFCKDFPGFIPSLLGDLLEERQKVKKRMKATIDPIEKKLLDYRQRAIKILANSLYGYYGYAKARWYCKECAESVIAWGRQYLETTIREIEEKFGFKVLYADTDGFFATIPGADAETVKKKAKEFLDYINAKLPGLLELEYEGFYKRGLFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIVDRAIPFDEFDPAKHKYDAEYYIEKQVLPAVERILRAFGYRKEDLRYQKTRQVGLGARLKPKTLEHHHHHH

SEQ ID NO: 12 - protein, 781 aa, synthetic construct
  source 1..781: note = TgoRT
MVLDTDYITEDGKPVIRIFKKENGEFKIDYDRNFEPYLYALLKDDSAIEDVKKITAERHGTTVRVVRAEKVKKKFLGRPIEVWKLYFTHPQDVPAIMDKIKEHPAVVDIYEYDIPFAIRYLIDKGLIPMEGDEELKLLDFEIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRHESHAGGYIKEPERGLWENIVYLDFRSLYPSIIITHNVSPDTLNREGCEEYDVAPQVGHKFCKDFPGFIPSLLGDLLEERQKVKKRMKATIDPIEKKLLDYRQRAIKILANSLYGYYGYAKARWYCKECAESVIAWGRQYLETTIREIEEKFGFKVLYADTDGFFATIPGADAETVKKKAKEFLDYINAKLPGLLELEYEGFYKRGLFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIVDRAIPFDEFDPAKHKYDAEYYIEKQVLPAVERILRAFGYRKEDLRYQKTRQVGLGARLKPKTLEHHHHHH

SEQ ID NO: 13 - protein, 6 aa, synthetic construct
  source 1..6: note = histidine purification tag
HHHHHH

SEQ ID NO: 14 - protein, 11 aa, synthetic construct
  source 1..11: note = short peptide C-terminal tag
SEEDEEKEEDG

SEQ ID NO: 15 - protein, 7 aa, synthetic construct
  source 1..7: note = Tobacco etch virus protease
ENLYFQG

SEQ ID NO: 16 - protein, 5 aa, synthetic construct
  source 1..5: note = Enterokinase (EntK) cleavage site
DDDDK

SEQ ID NO: 17 - protein, 4 aa, synthetic construct
  source 1..4: note = Factor Xa (Xa) cleavage site
IEGR

SEQ ID NO: 18 - protein, 6 aa, synthetic construct
  source 1..6: note = Thrombin (Thr) cleavage site
LVPRGS

SEQ ID NO: 19 - protein, 8 aa, Homo sapiens
  source 1..8: note = human rhinovirus 3C protease cleavage site
LEVLFQGP

SEQ ID NO: 20 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 21 - protein, 775 aa, Pyrococcus sp.
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 22 - protein, 775 aa, Thermococcus sp.
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 23 - DNA, 1980 bp, synthetic construct
  source 1..1980: mol_type = other DNA; note = MMLV variant
acttggctgtctgatttccctcaggcgtgggccgaaacgggtggcatgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 24 - protein, 660 aa, synthetic construct
  source 1..660: note = MMLV variant
TWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 25 - protein, 773 aa, synthetic construct
  source 1..773: note = RTx_ (Tgo-RTX)
MVLDTDYITEDGKPVIRIFKKENGEFKIDYDRNFEPYLYALLKDDSAIEDVKKITAERHGTTVRVVRAEKVKKKFLGRPIEVWKLYFTHPQDVPAIMDKIKEHPAVVDIYEYDIPFAIRYLIDKGLIPMEGDEELKLLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRHESHAGGYIKEPERGLWENIVYLDFRSLYPSIIITHNVSPDTLNREGCEEYDVAPQVGHKFCKDFPGFIPSLLGDLLEERQKVKKRMKATIDPIEKKLLDYRQRAIKILANSLYGYYGYAKARWYCKECAESVIAWGRQYLETTIREIEEKFGFKVLYADTDGFFATIPGADAETVKKKAKEFLDYINAKLPGLLELEYEGFYKRGLFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIVDRAIPFDEFDPAKHKYDAEYYIEKQVLPAVERILRAFGYRKEDLRYQKTRQVGLGARLKPKT

SEQ ID NO: 26 - protein, 775 aa, synthetic construct; identical to SEQ ID NO: 2
  source 1..775: note = Pfu-RTX

SEQ ID NO: 27 - protein, 775 aa, synthetic construct; identical to SEQ ID NO: 3
  source 1..775: note = Pfu-RTX

SEQ ID NO: 28 - protein, 774 aa, synthetic construct
  source 1..774: note = Targ-RTX
MILAADYITKDGKPIVRIFKKENGEFKIELDPHFRPYLYALLRDDSAIEEIMQIKGERHGKTVRIVDAIKVKKKFLRRPVEVWKLIFEHPQDVPAMMGKIRSHPAVVDIYEYDIPFAIRYLIDKGLVPMEGEEDLKLLAFDIETFYHEGDEFGKGEIIMISYADDEEAGVITWKRINLPYVHVVSNEREMIKRFVQIIKEKDPDVIITYNGDNFDLPYLIKRAEKLGVRLLLGRDKEHPEPKIQRMGDSFAVEIKGRIHFDLFPVVRRTVNLPTYTLEAVYETVLGKQKTKLGAEEIAAIWETEEGMKKLAQYSMEDAKATYELGREFFPMEAELAKVIGQSVWDVSRSSTGNLVEWYMLRVAYERNELAPNKPSDEEYKRRLHTTHIGGYIKEPERGLWGNIVYLDFRSLYPSIIVTHNVSPDTLEREGCQDYEVAPIVGYRFCKDFSGFIPSILENLIETRQEVKKRMKSTTDPVERKMLDYRQRALKILANSLYGYQGYPKARWYSKECAESVIAWGRHYLEMSIREIEEKFGFKVLYADTDGFYATIPGEKPDNIKKKAKEFLDYINSKLPGLLELEYEGFYLRGLFVTKKRYAVIDEDGRITTRGLEVVRRDWSEIAKETQAKVLEAILREGSVEKAVEIVKSVVERIAKYKVPLEKLVIHKQITRELKDYKAIGPHVAIAKRLAAKGIKVKPGTIISYIVLKGGGKIVDRVVLLTEYDPRKHKYDPDYYIDKQVLPAVLRILEAFGYKKEDLRYQRSKQTGLEARLRR

SEQ ID NO: 29 - protein, 774 aa, synthetic construct
  source 1..774: note = Targ-RTX
MILAADYITKDGKPIVRIFKKENGEFKIELDPHFRPYLYALLRDDSAIEEIMQIKGERHGKTVRIVDAIKVKKKFLRRPVEVWKLIFEHPQDVPAMMGKIRSHPAVVDIYEYDIPFAIRYLIDKGLVPMEGEEDLKLLAFAIATFYHEGDEFGKGEIIMISYADDEEAGVITWKRINLPYVHVVSNEREMIKRFVQIIKEKDPDVIITYNGDNFDLPYLIKRAEKLGVRLLLGRDKEHPEPKIQRMGDSFAVEIKGRIHFDLFPVVRRTVNLPTYTLEAVYETVLGKQKTKLGAEEIAAIWETEEGMKKLAQYSMEDAKATYELGREFFPMEAELAKVIGQSVWDVSRSSTGNLVEWYMLRVAYERNELAPNKPSDEEYKRRLHTTHIGGYIKEPERGLWGNIVYLDFRSLYPSIIVTHNVSPDTLEREGCQDYEVAPIVGYRFCKDFSGFIPSILENLIETRQEVKKRMKSTTDPVERKMLDYRQRALKILANSLYGYQGYPKARWYSKECAESVIAWGRHYLEMSIREIEEKFGFKVLYADTDGFYATIPGEKPDNIKKKAKEFLDYINSKLPGLLELEYEGFYLRGLFVTKKRYAVIDEDGRITTRGLEVVRRDWSEIAKETQAKVLEAILREGSVEKAVEIVKSVVERIAKYKVPLEKLVIHKQITRELKDYKAIGPHVAIAKRLAAKGIKVKPGTIISYIVLKGGGKIVDRVVLLTEYDPRKHKYDPDYYIDKQVLPAVLRILEAFGYKKEDLRYQRSKQTGLEARLRR

SEQ ID NO: 30 - protein, 774 aa, synthetic construct; identical to SEQ ID NO: 7
  source 1..774: note = KOD-RTXKOD-RTX

SEQ ID NO: 31 - protein, 774 aa, Thermococcus sp.
  source 1..774: note = Thermococcus argininiproducens
MILAADYITKDGKPIVRIFKKENGEFKIELDPHFRPYIYALLRDDSAIEEIMQIKGERHGKTVRIVDAIKVKKKFLRRPVEVWKLIFEHPQDVPAMRGKIRSHPAVVDIYEYDIPFAKRYLIDKGLVPMEGEEDLKLLAFDIETFYHEGDEFGKGEIIMISYADDEEAGVITWKRINLPYVHVVSNEREMIKRFVQIIKEKDPDVIITYNGDNFDLPYLIKRAEKLGVRLLLGRDKEHPEPKIQRMGDSFAVEIKGRIHFDLFPVVRRTVNLPTYTLEAVYETVLGKQKTKLGAEEIAAIWETEEGMKKLAQYSMEDAKATYELGREFFPMEAELAKVIGQSVWDVSRSSTGNLVEWYMLRVAYERNELAPNKPSDEEYKRRLRTTYIGGYVKEPERGLWGNIVYLDFRSLYPSIIVTHNVSPDTLEREGCQDYEVAPIVGYRFCKDFSGFIPSILENLIETRQEVKKRMKSTTDPVERKMLDYRQRALKILANSYYGYQGYPKARWYSKECAESVTAWGRHYIEMSIREIEEKFGFKVLYADTDGFYATIPGEKPDNIKKKAKEFLDYINSKLPGLLELEYEGFYLRGFFVTKKRYAVIDEDGRITTRGLEVVRRDWSEIAKETQAKVLEAILREGSVEKAVEIVKSVVERIAKYKVPLEKLVIHEQITRELKDYKAIGPHVAIAKRLAAKGIKVKPGTIISYIVLKGGGKISDRVVLLTEYDPRKHKYDPDYYIDNQVLPAVLRILEAFGYKKEDLRYQRSKQTGLEAWLRR

(19) United States
(12) Patent Application Publication
(10) Pub. No.: US 2026/0152775 A1
(43) Pub. Date: Jun. 4, 2026

(54) ENGINEERED NON-STRAND DISPLACING FAMILY B POLYMERASES FOR REVERSE TRANSCRIPTION AND GAP-FILL APPLICATIONS

(71) Applicant: 10X Genomics, Inc., Pleasanton, CA (US)

(72) Inventors: Derek Hunter Vallejo, Pleasanton, CA (US); Ruijie Zhang, Milpitas, CA (US)

(73) Assignee: 10X Genomics, Inc., Pleasanton, CA (US)

(21) Appl. No.: 19/391,813

(22) Filed: Nov. 17, 2025

Related U.S. Application Data
(63) Continuation of application No. PCT/US2024/030100, filed on May 17, 2024.
(60) Provisional application No. 63/467,541, filed on May 18, 2023.

(51) Int. Cl. C12P 19/34; C12N 9/12; C40B 70/00

(52) U.S. Cl. CPC C12P 19/34; C12N 9/1252; C12Y 207/07007; C40B 70/00

(57) ABSTRACT

The present disclosure relates generally to engineered nucleic acid processing enzymes, based on DNA polymerases (e.g., engineered family B polymerases), and derivatives thereof having reverse transcriptase activity and substantially lacking strand displacement activity; kits comprising the engineered family B polymerases; and methods of generating and using the engineered family B polymerases.

### CROSS REFERENCE

This application is a bypass Continuation of International Patent Application No. PCT/US2024/030100, filed May 17, 2024, which claims priority from U.S. Provisional Patent Application No. 63/467,541, filed May 18, 2023. The entire contents of these applications are hereby incorporated by reference in their entirety for all purposes.

### SEQUENCE LISTING

The present application contains a Sequence Listing which is hereby incorporated by reference in its entirety. Said Sequence Listing xml file was created on Jul. 26, 2024, is 51,300 bytes in size, and is named 131488-0232_Sequence_Listing.xml.

### TECHNICAL FIELD

The present disclosure relates to the fields of molecular biology, cell biology, biochemistry, and diagnostics, as they pertain to genetic engineering of reverse transcriptase (RT) enzymes for the reverse transcription of nucleic acid molecules.

### BACKGROUND

The following description of the background of the present technology is provided simply as an aid in understanding the present technology and is not admitted to describe or constitute prior art to the present technology.

The discovery of reverse transcriptase (RT) in the 1970's revolutionized the understanding of eukaryotic biology by demonstrating that genetic information did not flow unidirectionally from DNA to RNA to proteins. Rather, the genetic information could also flow in the reverse direction from RNA back to DNA. The ability to convert mature mRNA back into cDNA, without the introns present in genomic DNA is critical for obtaining information in a wide variety of biomedical contexts, including diagnostics, prognostics, biotechnology, and forensic biology. Since then, RT enzymes (RTs) have become ubiquitous tools in molecular biology driving enabling technologies such as next-generation RNA-Sequencing, Maxam-Gilbert sequencing and chain-termination methods, or de novo sequencing methods including shotgun sequencing and bridge PCR, or next-generation methods including polony sequencing, 454 pyrosequencing, Illumina sequencing, SOLiD sequencing, Ion Torrent semiconductor sequencing, HeliScope single molecule sequencing, SMRT® sequencing.

RT enzymes were initially found in retroviruses such as Moloney murine leukemia virus (MMLV)). It is now clear that RTs are present in other microorganisms, including transposable elements, where RTs are responsible for converting the RNA genome of these organisms into DNA to facilitate the integration of the microorganisms into a host's chromosome. All known natural RTs are derived from a shared common ancestor. Generally, RTs are mesophilic enzymes that function best at moderate temperatures ranging from 20° C. to 45° C. The mesophilic nature of RTs is problematic for in vitro amplification reactions because RNAs tend to adopt stable secondary structures at lower temperatures resulting in inefficient reverse transcription reactions at these low to moderate temperatures. In addition to the RNA secondary structures, RT reactions and amplification reactions also fail because biological samples from which nucleic acids are extracted often contain additional compounds that are inhibitory to reverse transcription and/or amplification reactions. This inhibition is particularly problematic when the volume of an amplification reaction is very small (e.g., nanoliter), such as in single cell profiling reactions and additional methods where small reaction volumes are preferred.

An example of an additional method is RNA-templated ligation (RTL). RTK is used to analyze spatial heterogeneity of cells/analytes within a biological sample (e.g., a tissue). RTL and related methods utilize multiple oligonucleotides that target adjacent or nearby complementary sequences, and often require gap filling.

Accordingly, there is a need for improved reverse transcriptases with improved properties, such as improved efficiency, processivity, thermoreactivity, thermostability with and without strand displacement activity. The present disclosure addresses this need.

### SUMMARY OF THE PRESENT TECHNOLOGY

The present disclosure provides engineered recombinant Family-B polymerases (e.g., engineered family B polymerases; engineered DNA polymerase enzymes; engineered polymerases) that have the fidelity and thermostability of known DNA polymerases in combination with a reverse transcriptase activity, and a substantial lack of strand displacement activity or no detectable strand displacement activity. Further provided are methods of using the engineered family B polymerases to generate polymerized nucleic acid products; nucleic acid extension methods comprising the engineered family B polymerases; methods for determining a location of a target nucleic acid in a biological sample comprising the engineered family B polymerases; and methods of analyzing a sample comprising a nucleic acid molecule using the engineered family B polymerases.

One aspect of the present disclosure provides a method of producing a polymerized nucleic acid product, the method comprising, consisting of, or consisting essentially of: (a) contacting an engineered family B polymerase with a probe-hybridized nucleic acid template and deoxyribonucleotide triphosphates, wherein the probe-hybridized nucleic acid template comprises a first probe end hybridized to a first region and a second probe end hybridized to a second region, and an unhybridized region between the first region and the second region; and (b) generating an extended product by extending the first probe end in the unhybridized region. In some embodiments, the engineered family B polymerase comprises mutations that confer reverse transcriptase activity. In some embodiments, the nucleic acid template comprises RNA.

In some embodiments of the method of producing a polymerized nucleic acid product described herein: (a) the first probe end and the second probe end are of a same probe molecule; or (b) the first probe end and the second probe end are of different probe molecules. In some embodiments, the nucleic acid templates are in a biological sample.

In some embodiments, the biological sample comprises a cell or tissue sample. In that embodiment, the cell or tissue sample comprises a Formalin-Fixed Paraffin-Embedded (FFPE) sample, a formalin-fixed sample, a paraffin-embedded sample, a frozen sample, or a fresh sample.

In some embodiments of the method of producing a polymerized nucleic acid product described herein, the unhybridized region comprises a site of genetic variability.

In some embodiments of the method of producing a polymerized nucleic acid product described herein, the method further comprises ligating a 3′ end of the extension product to a 5′ end of the second probe end.

In some embodiments of the method of producing a polymerized nucleic acid product described herein, the method further comprises modifying the extension product or an amplification copy thereof, to incorporate a barcode. In that embodiment, the barcode comprises a spatial barcode. In that embodiment, the method is performed in a spatial location in the biological sample, and the spatial barcode identifies the spatial location. In that embodiment, the barcode comprises a single cell barcode. In that embodiment, the method is performed in a partitioned cell.

In some embodiments of the method of producing a polymerized nucleic acid product described herein, the mutations that confer reverse transcriptase activity comprise mutations to positions 38, 97, 118, 137, 382, 385, 390, 467, 494, 515, 522, 588, 665, 712, 736, and 769 corresponding to positions of SEQ ID NO: 1; or 38, 97, 118, 137, 381, 384, 389, 466, 493, 514, 521, 587, 664, 711, 735, and 768 corresponding to positions of SEQ ID NO: 10. In that embodiment, the mutations that confer reverse transcriptase activity comprise: 38L, 97M, 118I, 137L, 381H, 384H, 389I, 466R, 493L, 514I, 521L, 587L, 664K, 711V, 735K, and 768R corresponding to positions of SEQ ID NO: 10.

In some embodiments of the method of producing a polymerized nucleic acid product described herein, the engineered family B polymerase is selected from the group consisting of Pyrococcus furiosus (pfu) polymerase, Thermococcus gorgonarius polymerase (Tgo polymerase), a Thermococcus kodakarensis (KOD1) polymerase, a Thermococcus litoralis (VENT®) polymerase, a Pyrococcus sp. (Deep Vent) polymerase, a Thermococcus sp. (9°N) polymerase, or a Thermococcus argininiproducens (Targ) polymerase.

In some embodiments, the engineered family B polymerase has an amino acid sequence selected from the group consisting of: SEQ ID NO: 2, SEQ ID NO: 3, SEQ ID NO: 4, SEQ ID NO: 5, SEQ ID NO: 11, SEQ ID NO: 12, SEQ ID NO: 25, SEQ ID NO: 26, SEQ ID NO: 27, SEQ ID NO: 28, SEQ ID NO: 29, and SEQ ID NO: 30. In that embodiment, the engineered family B polymerase has an amino acid sequence selected from the group consisting of: SEQ ID NO: 12, SEQ ID NO: 25, SEQ ID NO: 26, SEQ ID NO: 28, and SEQ ID NO: 30. In another embodiment, the engineered family B polymerase has an amino acid sequence of SEQ ID NO: 12 or SEQ ID NO: 25. In another embodiment, the engineered family B polymerase has an amino acid sequence of SEQ ID NO: 12.

In some embodiments of the method of producing a polymerized nucleic acid product described herein, the engineered family B polymerase further comprises one or more mutations that reduce or abolish exonuclease activity. In that embodiment, the one or more mutations that reduce or abolish exonuclease activity are at one or more of positions 2, 93, 141, 143, and 485, with respect to the positions of SEQ ID NO: 10.

In that embodiment, the one or more mutations that reduce or abolish exonuclease activity comprise mutations at positions 141 and 143 with respect to the positions of SEQ ID NO: 10, optionally wherein the mutations that reduce or abolish exonuclease activity comprise 141A and 143A with respect to the positions of SEQ ID NO: 10. In that embodiment, the engineered family B polymerase has an amino acid sequence selected from the group consisting of: SEQ ID NO: 3, SEQ ID NO: 4, SEQ ID NO: 5, SEQ ID NO: 11, SEQ ID NO: 27, and SEQ ID NO: 29. In that embodiment, the engineered family B polymerase has the amino acid sequence of SEQ ID NO: 11.

Another aspect of the present disclosure provides a nucleic acid extension method comprising, consisting essentially of, or consisting of: (a) contacting a target RNA molecule with (i) an engineered family B polymerase comprising mutations that confer reverse transcriptase activity, (ii) a first probe, and (iii) a second probe, where the first and second probe target non-adjacent regions of the target RNA molecule; and (b) incubating the target RNA molecule, the engineered family B polymerase, and the first and second probes under conditions in which the first and second probes hybridize to the target nucleic acid molecule; and (c) extending in a region between a 3′ end of the first probe and a 5′ end of the second probe to generate an extension product;

In some embodiments, the engineered family B polymerase comprises mutations: 38L, 97M, 118I, 137L, 381H, 384H, 389I, 466R, 493L, 514I, 521L, 587L, 664K, 711V, 735K, and 768R corresponding to positions of SEQ ID NO: 10. In that embodiment, the RNA molecule comprises a messenger RNA (mRNA) molecule.

In some embodiments, the first and/or the second probe comprises a capture sequence, and the method further comprises hybridizing the capture sequence to a barcode nucleic acid molecule.

In some embodiments of the nucleic acid extension method described herein, the barcode nucleic acid molecule is attached to a support; optionally the support is selected from the group consisting of an array, a bead, a gel bead, a microparticle, and a polymer.

Another aspect of the present disclosure provides a method for determining a location of a target nucleic acid in a biological sample, the method comprising, consisting essentially of, or consisting of: (a) contacting the biological sample with a plurality of first probe oligonucleotides and a plurality of second probe oligonucleotides, wherein: (i) the plurality of first probe oligonucleotides and the plurality of second probe oligonucleotides target a plurality of nucleic acids in the biological sample, (ii) each first probe and each second probe of the plurality comprise sequences that are substantially complementary to a target nucleic acid in the biological sample, and (iii) each second probe of the plurality comprises a capture probe domain sequence; where each first probe oligonucleotide and each second probe oligonucleotide of the plurality hybridize to sequences that are separated on a target nucleic acid of the plurality of nucleic acids, optionally where each first probe and each second probe of the oligonucleotide of the plurality are part of the same molecule or are part of different molecules; (c) extending each first probe oligonucleotide of the plurality using an engineered family B polymerase to generate an extended first probe oligonucleotide, thereby filling in a gap between the first probe oligonucleotide and the second probe oligonucleotide of the plurality, wherein the engineered family B polymerase comprises mutations that confer reverse transcriptase activity; (d) ligating the extended first probe oligonucleotide and the second probe oligonucleotide of the plurality, thereby creating a ligated product; (e) releasing the ligated product from the target nucleic acid; (f) contacting the biological sample with a substrate comprising a plurality of capture probes, wherein each capture probe of the plurality of capture probes comprises: (i) a spatial barcode and (ii) a capture domain, wherein the capture domain comprises a sequence that is complementary to all or a portion of the capture probe domain of the second probe oligonucleotide; and (g) hybridizing the ligation product to the capture domain of the capture probe affixed to the substrate.

In some embodiments of the method for determining a location of a target nucleic acid in a biological sample described herein, the method further comprises: (h) determining (i) all or a part of the sequence of extended first probe oligonucleotide, or a complement thereof, and (ii) all or a part of the sequence of the spatial barcode, or a complement thereof, and using the determined sequence of (i) and (ii) to identify the location of the analyte in the biological sample.

In some embodiments, the ligating the extended first probe to the second probe utilizes a ligase. In some embodiments, optionally the ligase: (a) comprises a family B ligase; (b) is selected from the group consisting of T4 DNA ligase, T4 RNA ligase, Chlorella virus DNA ligase, Paramecium bursaria Chlorella virus 1 DNA ligase I (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase; or (c) comprises a single stranded DNA ligase, or an Archaeal RNA ligase.

In some embodiments of the method for determining a location of a target nucleic acid in a biological sample described herein, each first probe oligonucleotide of the plurality and each second probe oligonucleotide of the plurality hybridize to nucleic acid sequences of a target nucleic acid that are: (a) about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other; or (b) 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900, or 1000 nucleotides away from each other.

In that embodiment, each first probe oligonucleotide of the plurality and each second probe oligonucleotide of the plurality hybridize to nucleic acid sequences of a target nucleic acid that are: (a) at least about 1-100, at least about 1-90, at least about 1-80, at least about 1-70, at least about 1-60, at least about 1-50, at least about 1-40, at least about 1-30, at least about 1-20, at least about 1-10, at least about 1-9, at least about 1-8, at least about 1-7, at least about 1-6, at least about 1-5, at least about 1-4, at least about 1-3, at least about 1-2 nucleotides apart; or (b) 1-100, 1-90, 1-80, 1-70, 1-60, 1-50, 1-40, 1-30, 1-20, 1-10, 1-9, 1-8, 1-7, 1-6, 1-5, 1-4, 1-3, or 1-2 nucleotides apart.

In some embodiments of the method for determining a location of a target nucleic acid in a biological sample described herein, the method further comprises extending a 3′ end of the capture probe using the ligation product.

In some embodiments of the method for determining a location of a target nucleic acid in a biological sample described herein, the determining step (h) comprises amplifying all or part of the ligation product using the engineered family B polymerase. In that embodiment, the amplifying amplifies (h) all or part of sequence of the ligation product, or a complement thereof, and (ii) the sequence of the spatial barcode, or a complement thereof.

Another aspect of the present disclosure provides a method of analyzing a sample comprising a target nucleic acid molecule, the method comprising, consisting essentially of, or consisting of: (a) providing: (i) a cell or nuclei sample comprising the target nucleic acid molecule, wherein the target nucleic acid molecule comprises a first target region and a second target region, optionally wherein the first target region is adjacent to the second target region; (ii) a first probe comprising a first probe sequence, and optionally another probe sequence, where the first probe sequence of the first probe is complementary to the first target region of the nucleic acid molecule; and (iii) a second probe comprising a second probe sequence, wherein the second probe sequence of the second probe is complementary to the second target region of the target nucleic acid molecule; (b) subjecting the sample to conditions sufficient to hybridize the first probe to the first target region and the second probe to the second target region, where the first target region and the second target region are non-adjacent; (c) partitioning a cell or nuclei of the cell or nuclei sample into a partition, generating an extension product from the first probe by extending between the first probe and the second probe by contacting the first probe with an engineered family B polymerase comprising mutations that confer reverse transcriptase activity; ligating the extension product to the second probe to generate a ligation product; denaturing the ligation product to remove the target nucleic acid molecule; and modifying the ligation product or an extension product thereof to incorporate a partition-specific barcode.

In some embodiments of the method of analyzing a sample comprising a target nucleic acid molecule described herein, the method further comprises: (h) determining (i) all or a part of the sequence of the extension product, or a complement thereof, and (ii) all or a part of the sequence of the spatial barcode, or a complement thereof, and using the determined sequence of (i) and (ii) to identify the location of the analyte in the biological sample.

In some embodiments, the partition comprises a single cell, a single nucleus, nucleic acids from a single cell, single cell nuclei, or a combination thereof.

In some embodiments, when a partition comprises multiple cells, the cells comprise any suitable barcode and/or index that permits computationally identifying nucleic acids that originated from a single cell and/or nucleus.

In some embodiments of the method of analyzing a sample comprising a target nucleic acid molecule described herein, the first probe, the second probe, or the first and second probes comprise additional sequences selected from probe specific barcode sequences, UMI, or any further sequences for nucleic acid processing and sequencing library generation.

In some embodiments of the method of analyzing a sample comprising a target nucleic acid molecule described herein: steps (a), (b) and (d) are conducted in bulk, prior to (c) partitioning; or steps (a) and (b) are conducted in bulk, and (d) is conducted after (c) partitioning; or in step (e), the ligating the extension product utilizes a ligase, optionally wherein the ligase: (i) comprises a family B ligase; (ii) is selected from the group consisting of T4 DNA ligase, T4 RNA ligase, Chlorella virus DNA ligase, Paramecium bursaria Chlorella virus 1 DNA ligase I (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase; or (iii) comprises a single stranded DNA ligase, or an Archaeal RNA ligase.

In some embodiments of the method of analyzing a sample comprising a target nucleic acid molecule described herein, the partition is a droplet, a well, a cell and/or a nucleus.

In some embodiments of the method of analyzing a sample comprising a target nucleic acid molecule described herein, the first target region is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second target region.

In some embodiments of the methods described herein, the engineered family B polymerase comprises an amino acid sequence that has: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 11, SEQ ID NO: 12, SEQ ID NO: 25, SEQ ID NO: 26, SEQ ID NO: 27, SEQ ID NO: 28, SEQ ID NO: 29, or SEQ ID NO: 30; (b) at least 95% identity to the amino acid sequence of SEQ ID NO: 11, SEQ ID NO: 12, SEQ ID NO: 25, SEQ ID NO: 26, SEQ ID NO: 27, SEQ ID NO: 28, SEQ ID NO: 29, or SEQ ID NO: 30; (c) at least 97% identity to the amino acid sequence of SEQ ID NO: SEQ ID NO: 11, SEQ ID NO: 12, SEQ ID NO: 25, SEQ ID NO: 26, SEQ ID NO: 27, SEQ ID NO: 28, SEQ ID NO: 29, or SEQ ID NO: 30; or (d) 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 11, SEQ ID NO: 12, SEQ ID NO: 25, SEQ ID NO: 26, SEQ ID NO: 27, SEQ ID NO: 28, SEQ ID NO: 29, or SEQ ID NO: 30.

In some embodiments of the method of analyzing a sample comprising a target nucleic acid molecule described herein, the sample is fixed.

In some embodiments of any of the methods described herein, the extending is performed at a temperature of between about 42° C. and about 55° C., optionally wherein the extending is performed at a temperature of between about 48° C. and about 53° C.

Another aspect of the present disclosure provides a method for analyzing a target nucleic acid in a biological sample, the method comprising, consisting essentially of, or consisting of: (a) contacting the biological sample with: (i) a first probe comprising a first probe sequence, and optionally another probe sequence, wherein the first probe sequence of the first probe is complementary to a first target region of the nucleic acid molecule, and wherein the first probe sequence comprises a first reactive moiety; and (ii) a second probe comprising a second probe sequence, wherein the second probe sequence of the second probe is complementary to a second target region of the nucleic acid molecule, and wherein the second probe sequence comprises a second reactive moiety; (b) hybridizing the first probe to the first target region and the second probe to the second target region, such that the first target region is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second target region, optionally wherein the first probe and the second probe are part of the same molecule or part of different molecule; (c) generating an extended first probe by contacting the first probe with an engineered family B polymerase comprising mutations that confer reverse transcriptase activity to generate a probe-linked nucleic acid molecule, thereby filling a gap between the first region and the second region; (d) ligating the extended first probe to the second probe to repair a residual nick between the probe-linked nucleic acid molecule and the second probe, and optionally releasing the probe-linked nucleic acid molecule from the target nucleic acid; (e) contacting the probe-linked nucleic acid molecule with a substrate comprising a plurality of capture probes to hybridize the probe-linked nucleic acid molecule to a capture domain of the capture probe which is affixed to the substrate; (f) further processing the hybridized probe-linked nucleic acid molecule to generate a sequencing library; (g) determining sequences of probe-linked nucleic acid molecules in the sequencing library or a complement thereof, and (h) using the determined sequences to identify the location of the target nucleic acid sequence in the biological sample.

In some embodiments, each second probe comprises a capture probe domain sequence. In some embodiments, the biological sample is fixed to a solid support. In that embodiment, the solid support is a slide, and the method determines spatial position of the target nucleic acids in the biological sample.

In some embodiments of the method for analyzing a target nucleic acid in a biological sample described herein, the engineered family B polymerase has reverse transcriptase activity and substantially lacks strand displacement activity; Optionally, in some embodiments, the engineered family B polymerase comprises a Pyrococcus furiosus (pfu) polymerase, a Thermococcus gorgonarius polymerase (Tgo polymerase), a Thermococcus kodakarensis polymerase, a Thermococcus litoralis (VENT®) polymerase, a Pyrococcus sp. (Deep Vent) polymerase, Thermococcus sp. (9°N) polymerase (SEQ ID NO: 22), or a Thermococcus argininiproducens (Targ) polymerase.

In some embodiments of the method for analyzing a target nucleic acid in a biological sample, the first probe, the second probe, or the first and second probes comprise additional sequences selected from probe specific barcode sequences, UMI, or any further sequences for nucleic acid processing and sequencing library generation.

In some embodiments of the method for analyzing a target nucleic acid in a biological sample, the engineered family B polymerase comprises an amino acid sequence that has: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 11, SEQ ID NO: 12, SEQ ID NO: 25, SEQ ID NO: 26, SEQ ID NO: 27, SEQ ID NO: 28, SEQ ID NO: 29, or SEQ ID NO: 30; (b) at least 95% identity to the amino acid sequence of SEQ ID NO: 11, SEQ ID NO: 12, SEQ ID NO: 25, SEQ ID NO: 26, SEQ ID NO: 27, SEQ ID NO: 28, SEQ ID NO: 29, or SEQ ID NO: 30; or (c) at least 97% identity to the amino acid sequence of SEQ ID NO: 11, SEQ ID NO: 12, SEQ ID NO: 25, SEQ ID NO: 26, SEQ ID NO: 27, SEQ ID NO: 28, SEQ ID NO: 29, or SEQ ID NO: 30.

In some embodiments, the engineered family B polymerase comprises the amino acid sequence set forth in SEQ ID NO: 2-5, 11, 12, and 25-30; or an amino acid sequence having at least 90% sequence identity to the amino acid sequence set forth in SEQ ID NO: 2-5, 11, 12, and 25-30.

In some embodiments of the method for analyzing a target nucleic acid in a biological sample, the ligase: (a) comprises a family B ligase; (b) is selected from the group consisting of T4 DNA ligase, T4 RNA ligase, Chlorella virus DNA ligase, Paramecium bursaria Chlorella virus 1 DNA ligase I (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase; or (c) comprises a single stranded DNA ligase, or an Archaeal RNA ligase.

In some embodiments of the method for analyzing a target nucleic acid in a biological sample described herein, (c) is performed at a temperature of between about 42° C. and about 55° C., optionally wherein the extending is performed at a temperature of between about 48° C. and about 53° C.

In some embodiments of any of the methods described herein, the engineered family B polymerase: (a) substantially lacks strand displacement activity; or (b) displaces: (i) no more than 10, 9, 8, 7, 6, 5, 4, 3, 2, or 1 nucleotides; (ii) 1-2, 1-3, 1-4, 1-5, 1-6, 1-7, 1-8, 1-9, or 1-10 nucleotides; or (iii) 1, 2, 3, 4, 5, 6, 7, 8, 9 or 10 nucleotides; (iv) about 6 nucleotides; or (v) about 10 nucleotides.

Another aspect of the present disclosure provides a kit comprising an engineered family B polymerase (e.g., Tgo polymerase) comprising, consisting essentially of, or consisting of one or more mutations that confer reverse transcriptase activity and a ligase.

In some embodiments, the engineered family B polymerase (e.g., Tgo polymerase) comprises the amino acid sequence of SEQ ID NO: 11, SEQ ID NO: 12, or SEQ ID NO: 25.

In some embodiments, the kit further comprises dNTPs.

In some embodiments, the kit further comprises a first oligonucleotide probe designed to hybridize to a first target region and a second oligonucleotide probe designed to hybridize to a second target region, wherein the first and the second target regions are non-adjacent,

In some embodiments of the kit described herein, the first and the second region are separated by at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, or at least 200 nucleotides

Both the foregoing summary and the following description of the drawings and detailed description are exemplary and explanatory. They are intended to provide further details of the disclosure but are not to be construed as limiting. Other objects, advantages, and novel features will be readily apparent to those skilled in the art from the following detailed description of the disclosure.

### BRIEF DESCRIPTION OF THE DRAWINGS

FIG. 1 shows an overview of the assay used to test the engineered family B polymerases of the present disclosure using 100 bp Glyceraldehyde 3-phosphate dehydrogenase (GAPDH) as a template; a 30-bp FAM-labeled primer that can be extended by an engineered family B polymerase; and a 29 bp 5′ Phosho blocking oligonucleotide (blocking oligo) that can be displaced by an engineered family B polymerase having strand-displacement activity. The expected size of the full length product is 100 nucleotides (nt) in the absence of the blocking oligo and about 71 nt in the presence of the blocking oligo. Reverse transcriptase (RT) enzymes tested include a variant Moloney Murine Leukemia Virus (MMLV) reverse-transcriptase (e.g., a control enzyme); an engineered Thermococcus kodakarensis polymerase (KOD-RTX; Family B Engineered Polymerase), which shows no strand displacement activity (SEQ ID NO: 7, 9, 30); an engineered Thermococcus gorgonarius polymerase (Tgo-RTX), engineered to have mutations on Tgo backbone (SEQ ID NO: 11, 12, 25) that resulted in a Tgo variant enzyme with minimal strand displacement activity; and Bst 3.0 (a Family A Engineered Polymerase) with strong strand displacement activity and no exonuclease (exo) activity.

FIGS. 2A-B show chromatographs illustrating amplification products obtained using the control MMLV RT with RT reagents in the absence (FIG. 2A) or the presence (FIG. 2B) of a blocking oligo; and demonstrating that the control MMLV RT completely displaced the 29 bp blocking oligo without any issues because a full length product of about 100 nt is produced.

FIGS. 3A-B show chromatographs illustrating amplification products obtained using an engineered Tgo RT enzyme (Tgo RTX; SEQ ID NO: 11, 12, or 25) with RT reagent B in the absence (FIG. 3A) or the presence (FIG. 3B) of a blocking oligo; and demonstrating that the Tgo-RTx had minimal strand displacement activity. The Tgo-RTx amplified the FAM-labeled primer to generate a product of about 77 nt (the major peak is around 77 nt), which meant that it displaced about 6 nt of the blocking oligo before termination (expected start of the blocking oligo is around 71 nt). Tgo-RTx could not produce full length product (e.g., 100 nt) in the presence of a blocking oligo.

FIGS. 4A-B show chromatographs illustrating amplification products obtained using an engineered KOD RT enzyme (KOD RTX; SEQ ID NO: 7, 9, or 30) with RT reagent B in the absence (FIG. 4A) or the presence (FIG. 4B) of a blocking oligo; and unexpectedly demonstrating that the KOD-RTX had no strand displacement activity. The FAM-labeled primer was extended up to the start of the blocking oligo (70 nt) and the blocking oligo was expected to start at about 71 nt. The reverse transcriptase activity of KOD RTX was also not as efficient as the reverse transcriptase activity of the Tgo (e.g., multiple truncated products in the absence of a blocking oligo).

FIGS. 5A-B show chromatographs illustrating amplification products obtained using Bst 3.0 with RT reagent B in the absence (FIG. 5A) or the presence (FIG. 5B) of a blocking oligo; and demonstrating that Bst 3.0, as expected, strand displaced very well (e.g., the same size product was obtained in the absence and the presence of the blocking oligo). Bst 3.0 was not as good as the MMLV variant (control) shown in FIGS. 2A-B because of the presence of minor truncated products around the expected start of the blocking oligo.

FIGS. 6A-D show an amino acid sequence alignment of some engineered family B polymerases disclosed herein, highlighting the positions of the mutations contemplated by the present disclosure and showing that pfu and Targ may contain an amino acid insertion after position 380. In addition, Targ may contain two additional amino acid insertions after position 236 (EH).

FIG. 7 shows the percent sequence identity between sequences aligned in FIGS. 6A-D; and illustrates that the amino acid sequence of wild type pfu (SEQ ID NO: 1) is at least about 79% identical to the amino acid sequence of wild type KOD1 (SEQ ID NO: 7); at least 80% identical to the amino acid sequence of wild type Tgo, and at least about 70% to the amino acid sequence of wild type Targ. The percent identity matrix was created using Clustal2.1 at ebi.ac.uk/Tools/msa/clustalo/.

FIGS. 8A-B show an overview of the assay used to further demonstrate that Tgo-RTX and Tgo-RTXo had minimal strand displacing activity and were therefore suitable for gap filling reactions.

FIG. 8A shows the 259 bp 5′ end of Glyceraldehyde 3-phosphate dehydrogenase (GAPDH) that was used as a template; the 30-bp FAM-labeled primer that can be extended by Tgo-RTX or Tgo-RTXo; and a 29 bp 5′ Phosho blocking oligonucleotide (blocking oligo) that can be displaced by an enzyme having strand-displacement activity. The expected size of the full length product was 259 nucleotides (nt) in the absence of the blocking oligo and about 230 nt in the presence of the blocking oligo (see also FIG. 1).

FIG. 8B shows an exemplary chromatograph obtained from the assay of FIG. 8A showing a single amplification product that illustrates gap filling with limited strand displacement (e.g., size is between 231 nt and 258 nt).

FIGS. 9A-B show bar graphs quantifying amplification products obtained using various concentrations (0.125 uM, 0.250 uM, 0.500 uM, and 1.00 uM) of Tgo-RTX(exo⁻) (FIG. 9A) and Tgo-RT(exo⁺) (FIG. 9B) at a temperature of 37° C.; and demonstrating that a significant fraction of the products generated from the reactions were truncated products. “Fully displaced” means a full length product with full strand displacement and had the expected size of 259 nt. “Truncated products” were products with less than 230 nt (i.e., reaction terminated before the expected start of the blocking oligo). “Gapfill” referred to optimal products that had exactly 230 nt (i.e., no strand displacement). “Partial displaced” referred to products that had between 231 nt to 258 nt (i.e., limited strand displacement). “Truncated Probe” referred to products that were shorter than the FAM-primer (fewer than 30 nt), and “Probe” referred to products that were exactly the length of the FAM-primer (30 nt in length).

FIGS. 10A-B show the same assay as in FIGS. 9A-B performed at a temperature of 42° C. In reactions comprising Tgo-RTX(exo⁻) (FIG. 10A) the majority of products were “Partial Displaced” products (231 nt to 258 nt), but in reactions comprising Tgo-RTX(exo⁺) (FIG. 10B) most products were “Truncated Products” (less than 230 nt) and Gapfill (230 nt).

FIGS. 11A-B show the same assay as in FIGS. 9A-B performed at a temperature of 48° C. In reactions comprising Tgo-RTX(exo⁻) (FIG. 11A) the majority of products were “Partial Displaced” products (231 nt to 258 nt), but in reactions comprising Tgo-RTX(exo⁺) (FIG. 11B) most products were Gapfill (230 nt).

FIGS. 12A-B show the same assay as in FIGS. 9A-B performed at a temperature of 53° C. In reactions comprising Tgo-RTX(exo⁻) (FIG. 12A) the majority of products were “Partial Displaced” products (231 nt to 258 nt) at lower enzyme concentrations (0.125 uM and 0.250 uM) and “Fully displaced” at higher enzyme concentrations (0.500 uM and 1.00 uM). In reactions comprising Tgo-RTX(exo⁺) (FIG. 11B) most products were Gapfill (230 nt).

### DETAILED DESCRIPTION

It is to be appreciated that certain aspects, modes, embodiments, variations and features of the present methods are described below in various levels of detail in order to provide a substantial understanding of the present technology.

While various embodiments of the disclosure have been shown and described herein, it will be obvious to those skilled in the art that such embodiments are provided by way of example only. Numerous variations, changes, and substitutions may occur to those skilled in the art without departing from any inventions of the present disclosure. It should be understood that various alternatives to the embodiments of the invention described herein may be employed.

### I. Overview

The spatial position of a cell within a tissue can affect its functional characteristics and behavior. For example, the spatial position can affect the cell's morphology, differentiation, fate, viability, proliferation, behavior, cell-cell signaling, and intracellular signaling.

RNA-templated ligation (RTL), or simply templated ligation is a heterogeneity assay that was developed to provide nucleic acid analysis in single cell sequencing application, and/or the spatial and temporal information of a single cell within a tissue. Unlike techniques that rely on targeting a common transcript sequence such as, e.g., a poly(A) mRNA-like tail to target a particular analyte in a biological sample, RTL offers an alternative to indiscriminate targeted RNA capture through the utilization of multiple oligonucleotides that target adjacent or nearby complementary sequences. As such, RTL seeks to increase target-specific detection of an analyte through hybridization of multiple (e.g., at least two) oligonucleotides, or probes, that are ligated together to one oligonucleotide product that can be detected by a capture probe, on any support, e.g., a bead, for single cell analysis, or on a spatial array.

RTL can be used to detect targets that vary by as small as a single nucleotide (e.g., in the setting of a single nucleotide polymorphism (SNP)). RTL can also be used for targeted RNA capture to interrogate spatial gene expression in a sample (e.g., a fresh or a fixed tissue). Compared to poly(A) mRNA capture, targeted RNA capture is less affected by RNA degradation associated with fixation (e.g., FFPE). Targeted RNA capture is also less affected by RNA degradation associated with fixation when compared to methods that depend on oligo-dT capture and reverse transcription of mRNA. Further targeted RNA capture allows for sensitive measurement of specific genes of interest that otherwise might be missed with a whole transcriptomic approach. Targeted RNA capture with RTL can be used to capture a defined set of RNA molecules of interest, or it can be used at a whole transcriptome level, or anything in between. When combined with the spatial methods known in the art, the location and abundance of the RNA targets can be determined.

However, some embodiments of the RTL require gap filling when the hybridization of the two oligonucleotides creates a gap between the hybridized oligonucleotides. In that case a nucleic acid processing enzyme (e.g., a DNA polymerase or a reverse transcriptase) is needed to extend one of the oligonucleotides prior to ligation, which is required in RTL. In particular, for RTL-based gap fill, such as an RNA targeted ligation for SNP detection, an RT is needed that can fill in any gaps between adjacent RTL probes or oligonucleotides without displacing the probe down-stream. Yet, wild-type reverse transcriptases have varying levels of strand displacement activity, making them unsuitable for gap fill during an RTL reaction.

To overcome these limitations, described herein are engineered nucleic processing enzymes based on B-family DNA polymerase enzymes that have been engineered to perform a reverse transcriptase activity. DNA polymerases have high fidelity, high thermostability, and many are known to lack strand displacing activity or to have minimal strand displacing activity. In contrast to canonical reverse transcriptase (RT) enzymes, the engineered DNA polymerase enzymes described herein have RT activity but lack strand displacing activity or have minimal strand displacing activity.

Accordingly, the present disclosure provides engineered Family-B polymerases (i.e., engineered family B polymerases) that have the fidelity and thermostability of known DNA polymerases in combination with a reverse transcriptase activity; and substantially lack strand displacement activity. The family B polymerases were genetically engineered, via mutagenesis, to have the properties of a reverse transcriptase, while maintaining the DNA polymerase activity. These polymerase enzymes were engineered based on the amino acid sequence of an engineered T. kodakarensis polymerase (KOD-TRX). See e.g., Ellefson et al. Science, 352(6293):1590-3 (2016).

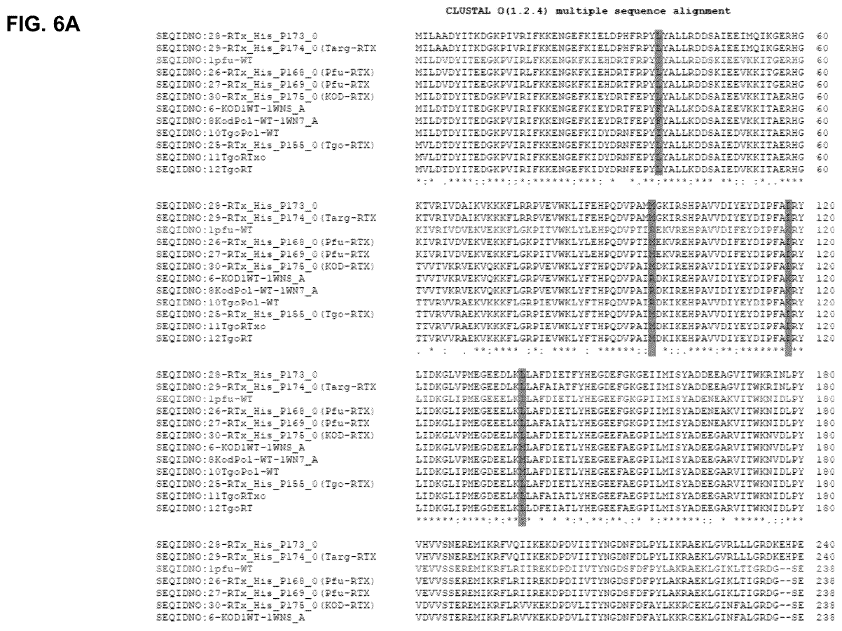

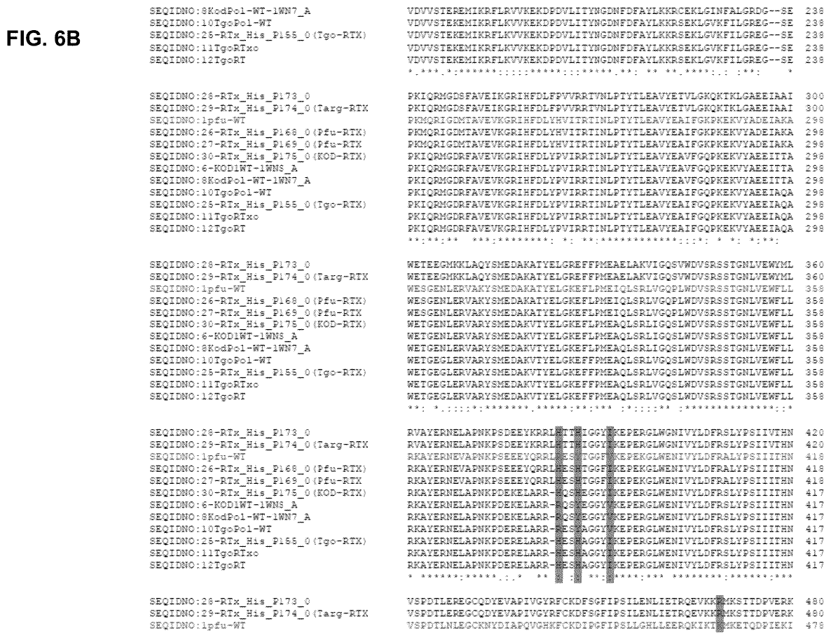

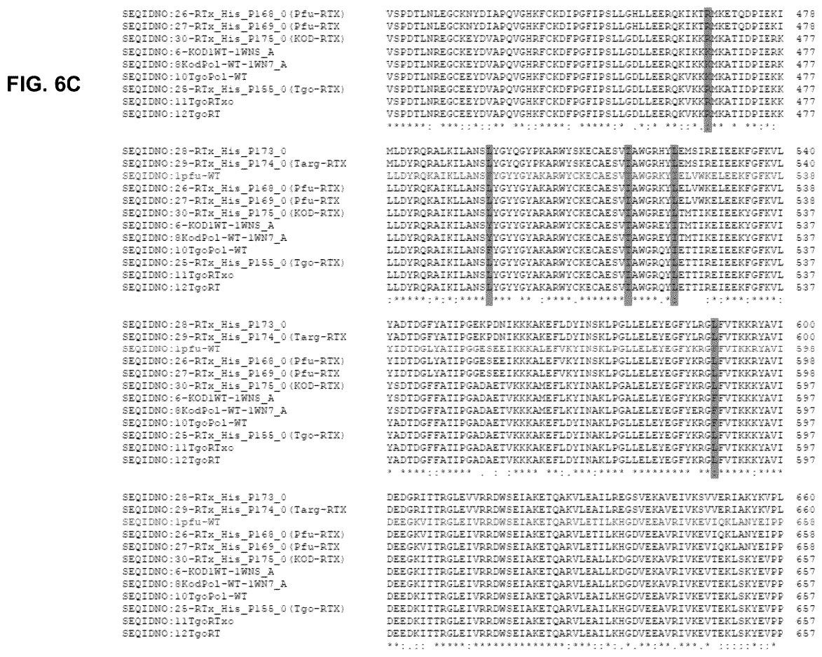

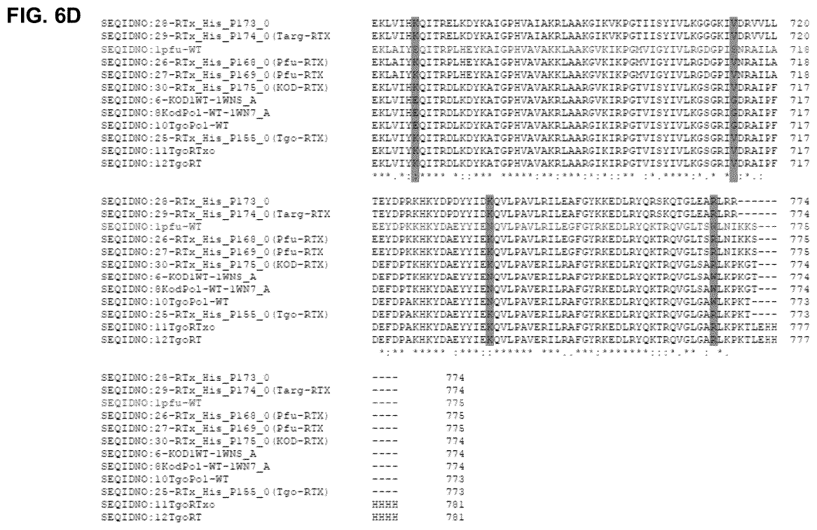

Family-B polymerases (polB) were selected because they have been widely adopted in modern molecular biology due to their hyperthermostability, processivity, and fidelity. However, Family B polymerase enzymes show little to no activity on RNA templates. Family-B polymerases (polB) contemplated by the present disclosure include, but are not limited to Pyrococcus furiosus (pfu) polymerase (SEQ ID NO: 1), Thermococcus gorgonarius polymerase (Tgo polymerase) (SEQ ID NO: 10), Thermococcus kodakarensis (KOD1) polymerase (SEQ ID NO: 6 or 8), Thermococcus litoralis (VENT®) polymerase (SEQ ID NO: 20), Pyrococcus sp. (Deep Vent) polymerase (SEQ ID NO: 21), Thermococcus sp. (9°N) polymerase (SEQ ID NO: 22), or Thermococcus argininiproducens (Targ) polymerase (SEQ ID NO: 31). The sequences of the exemplary engineered family B polymerases contemplated by the present disclosure are shown in FIGS. 6A-D.

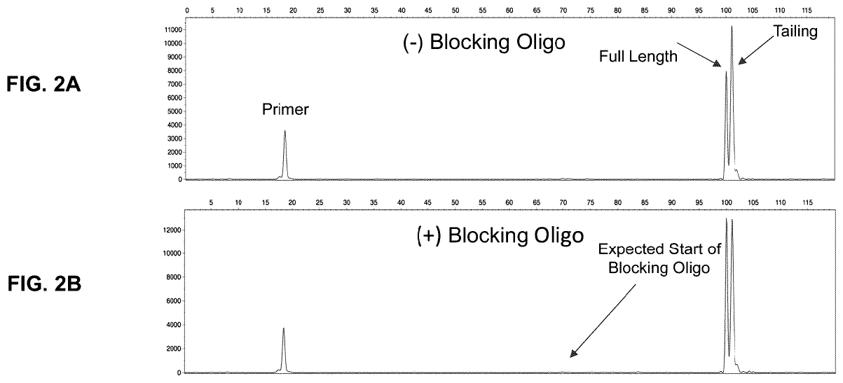

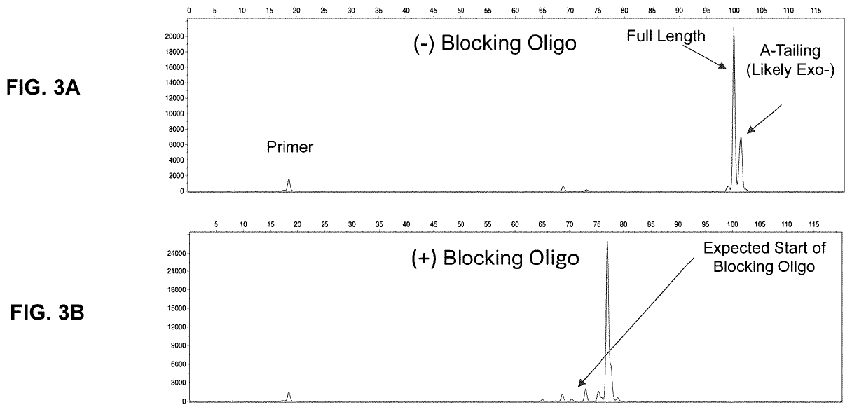

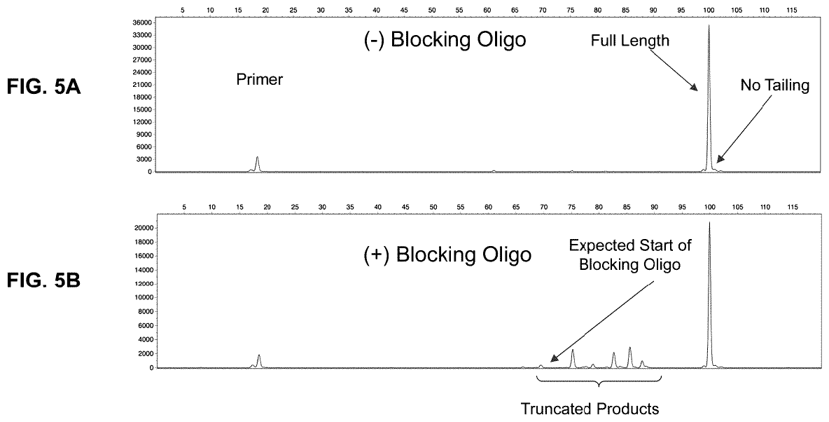

In contrast to a variant MMLV RT (control) enzyme (FIGS. 2A-B) and Bst 3.0 (FIG. 5A-B), which show strong strand displacement activity, engineered family B polymerases disclosed herein showed minimal to no strand displacement activity. For example, an engineered Thermococcus gorgonarius polymerase (Tgo polymerase) of the present disclosure exhibited a combination of reverse transcriptase activity, high thermostability, and minimal strand displacement activity (FIGS. 3A-B). For example, Tgo was able to amplify about 6 nt in the presence of a blocking oligo (71 nt (expected start of the blocking oligo) to 77 nt (final product)). In addition, the engineered Tgo reverse transcriptase described herein was more efficient and processive than a wild-type Tgo polymerase.

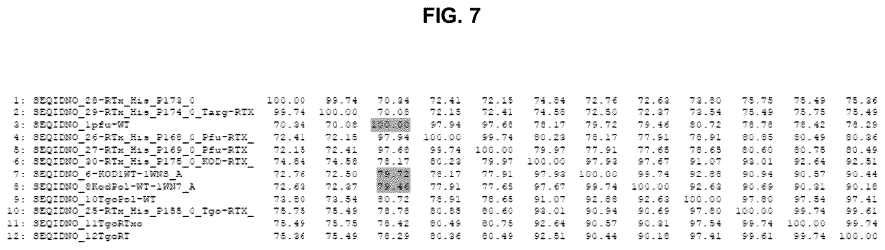

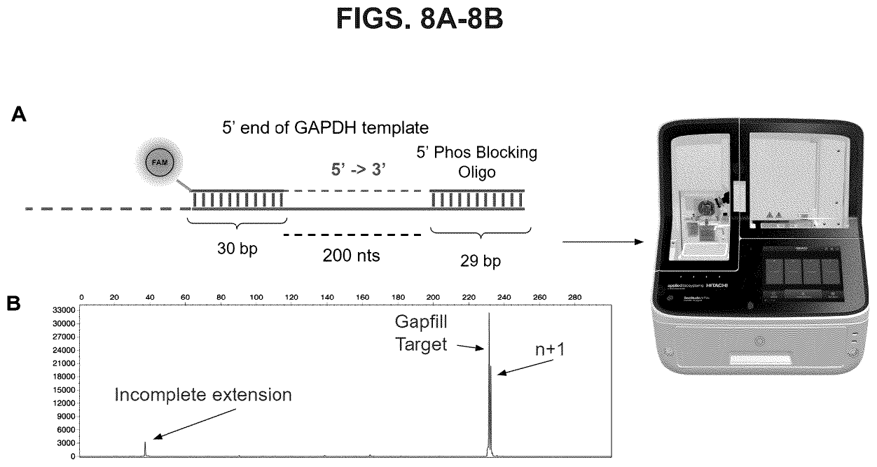

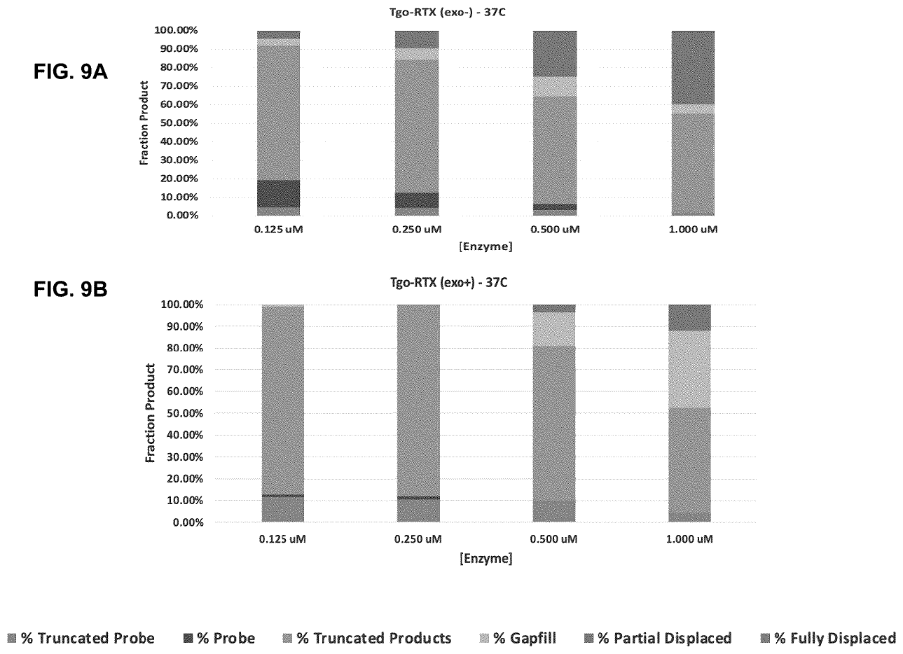

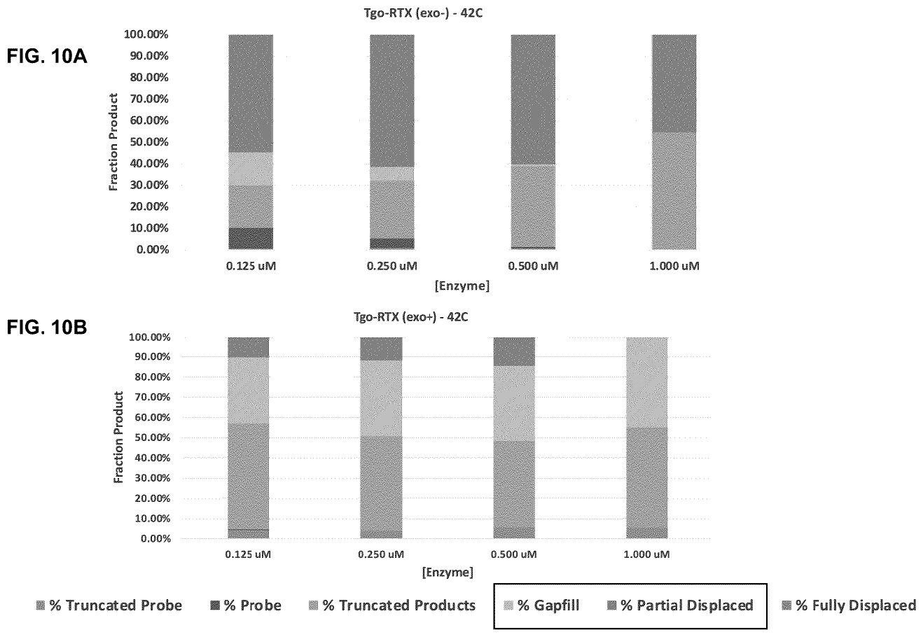

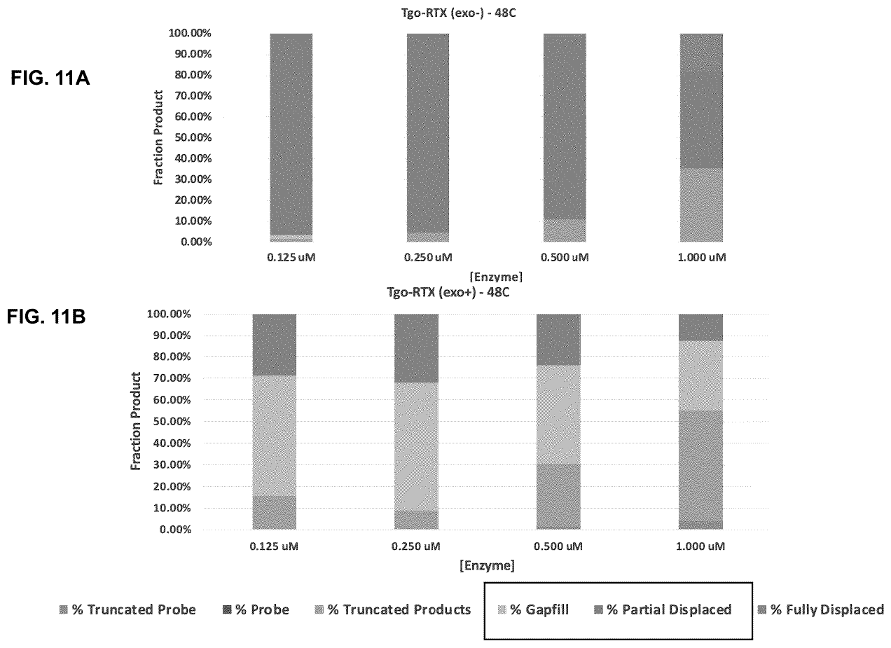

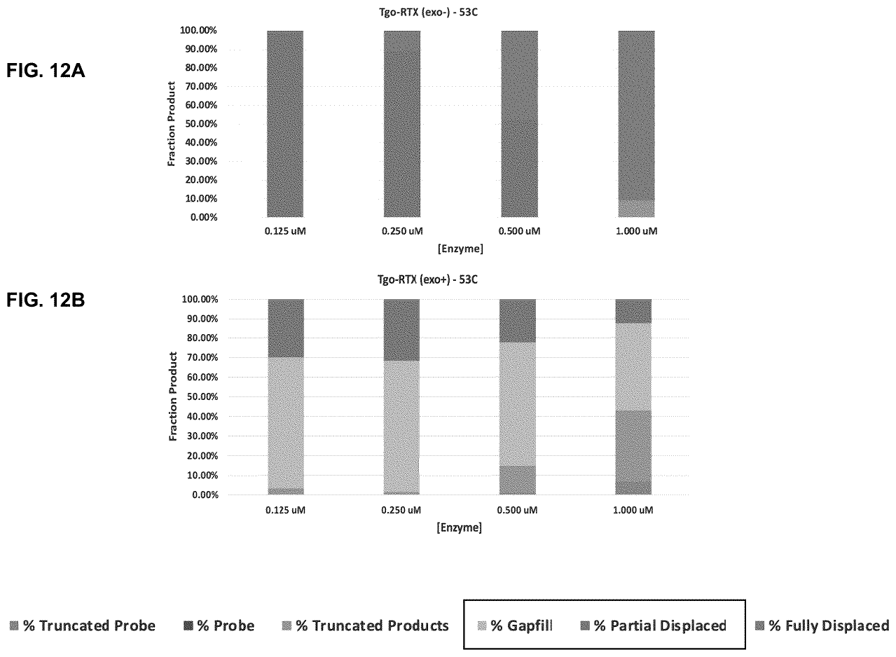

Unexpectedly, the non-strand displacing activity of the engineered Tgo polymerase was dependent on the concentration of the enzyme, the temperature of the reaction, and the exonuclease activity (FIGS. 8A-B, 9A-B, 10A-B, 11A-B, and 12A-B). For example, Tgo-RTX (exo⁻) generated products that were mostly “Truncated Products” (i.e., product length was less than 230 nt) at 37° C. (FIG. 9A); mostly “Partial Displaced” products (i.e., product length between 231 nt and 258 nt) at 42° C. (FIG. 10A), 48° C. (FIG. 11A); and 53° C. (FIG. 12A). At 53° C., the majority of products were surprisingly “Fully displaced” products (259 nt) when 0.500 μM or 1.000 μM Tgo-RTX (exo⁻) was used (FIG. 12A). Thus, the engineered Tgo Exo⁻ variant began to fully displace the blocking oligo at a concentration of about 0.250 μM (see also, 1.000 μM at 48° C.; FIG. 11A).

Tgo-RTX (exo⁺) also generated products that were mostly “Truncated Products” (i.e., product length was less than 230 nt) at 37° C. (FIG. 9B); mostly “Gapfill” products (i.e., product length 230 nt) at 42° C. (FIG. 10B), 48° C. (FIG. 11B); and 53° C. (FIG. 12B). “Gapfill” products were also generated when higher concentrations (e.g., 0.500 μM and 1.00 μM) of Tgo-RTX (exo⁺) were used at 37° C. (FIG. 9B). At 53° C., lower concentrations of exo⁺ gave nearly complete conversion to desired product (Gapfill (70%) or Partial displaced (25-40%).

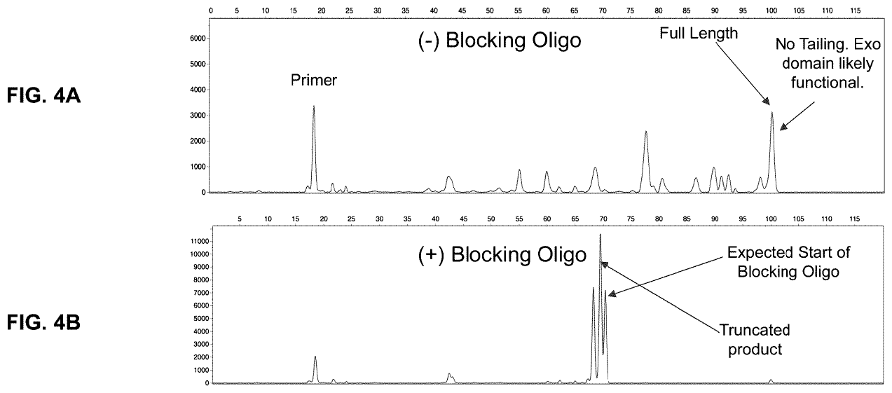

The engineered KOD also showed no strand displacement activity (FIGS. 4A-B).

In addition to improving gap-filling during RTL, the engineered family B polymerases of the present disclosure can also be used in a single-step amplification reaction to generate a nucleic acid amplification product (DNA) by first generating a cDNA from mRNA and then amplifying that cDNA using the single engineered reverse transcriptase polymerase enzyme of the present disclosure. This one-step reaction has many advantages. For example, a thermophilic reverse transcriptase enzyme with dual reverse transcriptase and DNA polymerase activity would: (1) render unnecessary the use of template switching oligonucleotides; (2) reduce the dependence on template switching for amplification reactions as found in spatial arrays and single cell transcriptomics assays, and (3) simplify and expedite any RT-PCR reactions known in the art.

Furthermore, the engineered family B polymerases (e.g., engineered recombinant Family-B polymerases; engineered DNA polymerase enzymes; engineered DNA polymerases; or engineered enzymes) disclosed herein can be used in any amplification schemes that require a reverse transcriptase and/or a DNA polymerase, including, but not limited to, Reverse Transcription Loop-mediated Isothermal Amplification (RT-Lamp), self-sustained sequence replication reaction (3SR) or nucleic acid sequence-based amplification (NASBA), transcription mediated amplification (TMA), Rolling circle amplification (RCA), Recombinase polymerase amplification (RPA), or helicase-dependent amplification (HAD).

The engineered family B polymerases (e.g., engineered recombinant Family-B polymerases; engineered DNA polymerase enzymes; engineered DNA polymerases; engineered enzymes) of the present disclosure are novel tools for overcoming the limitations associated with sequencing RNA templates and/or using RNA templates in a single cell analysis system, or in spatial array single cell transcriptomics assays, and/or RTL as disclosed herein.

### II. Engineered Reverse Transcriptases

### A. Polymerases Suitable for Engineering

In one aspect, the present disclosure provides an engineered family B polymerase (e.g., a nucleic acid processing enzyme) comprising, consisting essentially of, or consisting of an amino acid sequence having at least 75% sequence identity to the amino acid sequence of Pyrococcus furiosus (pfu) polymerase (SEQ ID NO: 1), Thermococcus gorgonarius polymerase (Tgo polymerase) (SEQ ID NO: 10), Thermococcus kodakarensis (KOD1) polymerase (SEQ ID NO: 6 or 8), Thermococcus litoralis (VENT®) polymerase (SEQ ID NO: 20), Pyrococcus sp. (Deep Vent) polymerase (SEQ ID NO: 21), Thermococcus sp. (9°N) polymerase (SEQ ID NO: 22), or Thermococcus argininiproducens (Targ) polymerase (SEQ ID NO: 31).

In some embodiments, the engineered family B polymerase has a reverse transcriptase activity and substantially lacks strand displacement amplification activity. Alternatively, the engineered family B polymerase can have reverse transcriptase activity and no detectable strand displacement activity. In particular, for RTL-based gap fill, such as an RNA targeted ligation for SNP detection, an RT is needed that can fill in any gaps between adjacent RTL probes or oligonucleotides without displacing the probe down-stream.

In some embodiments, the engineered family B polymerase has DNA, RNA, and DNA and RNA polymerase activity.

Archaeal Family-B polymerases (polB) have been widely adopted in modern molecular biology due to their hyperthermostability, processivity, and fidelity. Accordingly, polymerases suitable for engineering a reverse transcriptase enzyme as described herein are not limited to a Thermococcus gorgonarius (Tgo) polymerase, Thermococcus kodakarensis (KOD1), Thermococcus litoralis polymerase (SEQ ID NO: 20), Pyrococcus sp. (Deep Vent) polymerase, Thermococcus sp. (9°N) polymerase, or Thermococcus argininiproducens (Targ) polymerase. In some embodiments, polymerases suitable for engineering a reverse transcriptase of the present disclosure include, but are not limited to archaeal, bacterial, and eukaryotic polymerases. Polymerases include both DNA-dependent polymerases and RNA-dependent polymerases such as reverse transcriptases. At least five families of DNA-dependent DNA polymerases are known, although most fall into families A, B and C. There is little or no sequence similarity among the various families. Most family A polymerases are single chain proteins that can contain multiple enzymatic functions including polymerase activity, 3′ to 5′ exonuclease activity and 5′ to 3′ exonuclease activity. Family B polymerases typically have a single catalytic domain with a polymerase, and 3′ to 5′ exonuclease activity, as well as accessory factors. Family C polymerases are typically multi-subunit proteins with polymerizing activity and 3′ to 5′ exonuclease activity.

In some embodiments, the polymerase of the present disclosure is a B-type family DNA polymerase. B-type Family DNA polymerases include, but are not limited to, any DNA polymerase that is classified as a member of the Family B DNA polymerases. The Family B classification is based on structural similarity to E. coli DNA polymerase II and is also based on the presence of known and conserved regions referred to as motif A and motif B of the family B polymerases. B-type family polymerases include bacterial and bacteriophage polymerases. In some embodiments, the B-type family polymerase is E. coli DNA polymerase II; PRD1 DNA polymerase; phi29 DNA polymerase; M2 DNA polymerase; and T4 DNA polymerase. In some embodiments, the B-type family polymerase is an archaeal DNA polymerases such as Thermococcus litoralis DNA polymerase (Vent); Pyrococcus furiosus DNA polymerase; Sulfolobus solfataricus DNA polymerase; Thermococcus gorgonarius DNA polymerase (Tgo pol); Pyrodictium occultum DNA polymerase; Methanococcus voltae DNA polymerase; Thermococcus species TY; T. kodakarensis polymerase (KodPol); Sulfolobus acidocaldarius DNA polymerase; Thermococcus species 9° N-7 (Therminator™); or Thermococcus species 9°N.

In some embodiments, the polymerase is an Eukaryotic B-type family DNA polymerases selected from the group consisting of DNA polymerase alpha; Human DNA polymerase (alpha); S. cerevisiae DNA polymerase (alpha); S. pombe DNA polymerase I (alpha); Drosophila melanogaster DNA polymerase (alpha); Trypanosoma brucei DNA polymerase (alpha); DNA polymerase delta; Human DNA polymerase (delta); Bovine DNA polymerase (delta); S. cerevisiae DNA polymerase III (delta); S. pombe DNA polymerase III (delta); and Plasmodium falciparum DNA polymerase (delta).

DNA polymerases have a common overall structure that has been likened to a human right hand, with fingers, thumb, and palm subdomains. The palm subdomain contains motif A which in turn contains a catalytically active aspartic acid residue. In native DNA polymerases, motif A begins at an anti-parallel P-strand containing predominantly hydrophobic residues and is followed by a turn and an a-helix. In native DNA polymerases, motif A interacts with a next correct nucleotide via coordination with divalent metal ions that participate in the polymerization reaction. Motif B contains an alpha-helix with positive charges. Further characteristics of motif A and motif B are known in the art, for example, as set forth in Delarue et al., Protein Eng., 3: 461-467 (1990); Shinkai et al., J. Biol. Chem., 276: 18836-18842 (2001), and Steitz, T. A., J. Biol. Chem., 274:17395-17398 (1999).

In some embodiments, the polymerase is a family B polymerase comprising a motif A and a motif B conserved regions. The terms “motif A” and “motif B” are intended to be used in accordance with their known meaning in the art. The terms are used to refer to regions of structural homology in the nucleotide binding sites of B family and other polymerases. Motif A and motif B are conserved regions among polymerases involved in nucleotide binding and substrate specificity. In some embodiments, motif A refers specifically to amino acids 408-410 of SEQ ID NO: 10 (Wild type Tgo Pol), or a motif that includes amino acids 408-410 of SEQ ID NO: 10. In some embodiments, motif B refers specifically to amino acids 484-486 of SEQ ID NO: 10, or to the motif that includes amino acids 484-486 SEQ ID NO: 10. Functionally equivalent or homologous “motif A” and “motif B” regions of polymerases other than the ones described herein can be identified on the basis of amino acid sequence alignment and/or molecular modelling. Sequence alignments may be compiled using any of the standard alignment tools known in the art, such as for example BLAST or CLUSTAL W. An exemplary sequence alignment is shown in FIGS. 6A-D and an identity matrix showing the sequence homology/identity is shown in FIG. 7.

Other polymerases that can be engineered include, for example, those that are members of families identified as A, C, D, X, Y, and RT. The RT (reverse transcriptase) family of DNA polymerases includes, but is not limited to, retrovirus reverse transcriptases and eukaryotic telomerases. Exemplary RNA polymerases include, but are not limited to, viral RNA polymerases such as, T7 RNA polymerase; eukaryotic RNA polymerases, such as RNA polymerase I, RNA polymerase II, RNA polymerase III, RNA polymerase IV, and RNA polymerase V; and archaea RNA polymerase. Motif A is present in RNA polymerases and can be modified at specified positions to generate DNA polymerases. Conversely, DNA polymerases can be modified as disclosed herein to engineer an enzyme with RT activity.

In some embodiments, the engineered family B polymerase contemplated by the present disclosure comprises an amino acid sequence that has at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31. In some embodiments, the engineered family B polymerase described herein comprises 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 6, 8, 10, 20-22, or 31. In some embodiments, the engineered family B polymerase can also have at least 95% identity to the amino acid sequence of SEQ ID NO: 1, 6, 8, 10, 20-22, or 31. Alternatively, the engineered family B polymerase has at least 97% identity to the amino acid sequence of SEQ ID NO: 1, 10, 20-22, or 31.

The percent sequence identity, in the context of two or more nucleic acid or polypeptide sequences, refers to the number of residues or bases that are the same for a given alignment of two polypeptide or nucleic acid sequences. Sequences sharing a specified percentage of nucleotides or amino acid residues, respectively, that are the same, when compared and aligned for a given parameter such as maximum correspondence, as measured using one of the sequence comparison algorithms described below (or other algorithms available to persons of skill) or by visual inspection.

By convention, amino acid additions, substitutions, and deletions within an aligned reference sequence are all differences that may reduce the percent identity depending upon the parameters used to assess percent identity. Often, additions, substitutions, and deletions within an aligned reference sequence are evaluated in an equivalent manner. In some cases, length variation between two sequences resulting in one sequence having bases or residues beyond the N- or C-terminus or 5′ or 3′ end of the other sequence are discarded in sequence alignment, such that the aligned region is defined by the ends of the shorter or earlier ending sequence and amino acids extending beyond the N- or C-terminus of a polynucleotide or 5′ or 3′ end of the earlier terminating sequence have no effect on percent identity scoring for aligned regions. For example, by one calculation approach, alignment of a 105 amino acid long polypeptide to a reference sequence 100 amino acids long would have a 100% identity score if the reference sequence fully was contained as a consecutive ungapped segment within the longer polynucleotide with no amino acid differences. Under such an assessment, a single amino acid difference (addition, deletion or substitution) between the two sequences within the 100-amino acid span of the aligned reference sequence would mean the two sequences were 99% identical.

In contrast, “Substantially identical,” in the context of two nucleic acids or polypeptides (e.g., DNAs encoding a polymerase, or the amino acid sequence of a polymerase) refers to two or more sequences or subsequences that have at least about 60%, at least about 80%, at least about 90-95%, at least about 98%, at least about 99% or more nucleotide or amino acid residue identity, when compared and aligned for maximum correspondence, as measured using a sequence comparison algorithm, or by visual inspection. Such “substantially identical” sequences are typically considered to be “homologous,” without reference to actual ancestry. The “substantial identity” exists over a region of the sequences that is at least about 50 residues in length, at least about 100 residues, at least about 150 residues, or over the full length of the two sequences to be compared.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity over about 50, about 100, about 150 or more residues is routinely used to establish homology. Higher levels of sequence similarity, such as at least about 30%, about 40%, about 50%, about 60%, about 70%, about 80%, about 90%, about 95%, or about 99% or more, can also be used to establish homology.

Methods for determining sequence similarity percentages (e.g., BLAST protein (BLASTP) and nucleotide (BLASTN) using default parameters) are described herein and are generally available. For sequence comparison and homology determination, typically one sequence acts as a reference sequence to which test sequences are compared. When using a sequence comparison algorithm, test and reference sequences can be input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison are known to those skilled in the art.

In some embodiments, the engineered family B polymerase of the present disclosure comprises a mutation. In some embodiments, the engineered family B polymerase can comprise a substitution at a position corresponding to a position selected from 38, 97, 118, 137, 381, 384, 389I, 466, 493, 514, 521, 587, 664, 711, 735, or 768 in SEQ ID NO: 7, or any combination thereof, or the combination of all substitutions. Alternatively, the enzyme can comprise an amino acid substitution at positions corresponding to a position selected from selected from 38, 97, 118, 137, 381, 384, 389I, 466, 493, 514, 521, 587, 664, 711, 735, and 768 in SEQ ID NO: 6.

In some embodiments, the engineered family B polymerase comprises a substitution corresponding to any amino acid substitution selected from 38L, 97M, 118I, 137L, 381H, 384H, 389I, 466R, 493L, 514I, 521L, 587L, 664K, 711V, 735K, 768R, or any combination thereof, or the combination of all substitutions. FIGS. 6A-D show relevant corresponding positions for contemplated substitution as described herein. An identity matrix showing the homology/identity between the sequences is shown in FIG. 7.

In some embodiments of the engineered family B polymerase described herein, the engineered family B polymerase further comprises an amino acid substitution at any position corresponding to position I2, V93, D141, E143, A485 in SEQ ID NO: 7, or any combination thereof, or the combination of all substitutions. Optionally, in some embodiments, the substitutions are I2V, V93Q, D141A, E143A, A485L, or any combination thereof, or the combination thereof in SEQ ID NO: 7. In those embodiments, the engineered family B polymerase comprises the amino acid sequence of SEQ ID NO: 11, 12, 25, or 28-30.

In some embodiments, the engineered family B polymerase as described herein comprises one or more substitutions selected from the group consisting of an aspartic acid substitution at position 141; a glutamic acid substitution at position 143; an alanine substitution at position 485; a valine substitution at position 93; an arginine substitution at position 97; a tyrosine substitution at position 384; a valine substitution at position 389; a phenylalanine at position 493; a phenylalanine substitution at position 587; a glutamic acid substitution at position 664; a glycine substitution at position 711; a tryptophan substitution at position 768; an isoleucine substitution at position 2; an isoleucine substitution at position 38; a lysine substitution at position 118; a methionine substitution at position 137; an arginine substitution at position 381; a lysine substitution at position 466; a tyrosine substitution at position 514; an isoleucine substitution at position 521; and an asparagine substitution at position 735 of SEQ ID NO: 10.

In some embodiments, the engineered family B polymerase as described herein comprises one or more substitutions selected from the group consisting of an aspartic acid to alanine substitution at position 141 (D141A); a glutamic acid to alanine substitution at position 143 (E143A); an alanine to leucine substitution at position 485 (A485L); a valine to glutamine substitution at position 93 (V93Q); an arginine to methionine substitution at position 97 (R97M); a tyrosine to histidine substitution at position 384 (Y384H); a valine to isoleucine substitution at position 389 (V389I); a phenylalanine to leucine substitution at position 493 (F493L); a phenylalanine to leucine substitution at position 587 (F587L); a glutamic acid to lysine substitution at position 664 (E664K); a glycine to valine substitution at position 711 (G711V); a tryptophan to arginine substitution at position 768 (W768R); an isoleucine to valine substitution at position 2 (I2V); an isoleucine to leucine substitution at position 38 (I38L); a lysine to isoleucine substitution at position 118 (K118I); a methionine to leucine substitution at position 137 (M137L); an arginine to histidine substitution at position 381 (R381H); a lysine to arginine substitution at position 466 (K466R); a tyrosine to isoleucine substitution at position 514 (T514I); an isoleucine to leucine substitution at position 521 (I521L); and an asparagine to lysine substitution at position 735 (N735K) of SEQ ID NO: 10.

In some embodiments, the engineered family B polymerase comprises a substitution at positions 141 and 143 of SEQ ID NO: 1-12 and 20-31. In some embodiments, the polymerase domain comprises a substitution at position 141 of SEQ ID NO: 3-5, 11, and 20-31 and/or lacks proofreading activity.

In one embodiment, the engineered family B polymerase lacks proofreading activity (3′-5′ exonuclease). Methods for inactivating the exonuclease activity of an enzyme via genetic engineered disruption of the exonuclease domain are well known in the art. In some embodiments, the exonuclease deficient enzyme comprises D141A and E143A in any one of SEQ ID NO: 1-12 and 20-31. In some embodiments, the engineered family B polymerase has proofreading activity. In such an embodiment, the disclosed engineered family B polymerase shows at least two, at least three, or least four fold improvement in fidelity over existing reverse transcriptases. As used herein, the “exonuclease domain” refers to the amino acids of the polymerase that binds to the primer terminus in the editing mode for removing misincorporations. This mechanism is important for proofreading (3′-5′ exonuclease) and contributes to processivity. In some embodiments, as shown in FIG. 3A, the engineered Tgo-RTX disclosed herein showed A-tailing, which may indicate that the enzyme may lack or may have reduced exonuclease activity (e.g., likely Exo⁻).

In some embodiments of the engineered family B polymerase described herein, the engineered family B polymerase is an engineered Thermococcus kodakarensis (KOD1). In some embodiments, the wild-type KOD polymerase comprises the amino acid of SEQ ID NO: 6 or 8. In some embodiments, the engineered KOD1 comprises the amino acid sequence of SEQ ID NO: 7, 9, or 30.

In some embodiments of the engineered family B polymerase described herein, the engineered family B polymerase is an engineered Thermococcus argininiproducens (Targ) polymerase. In some embodiments, the wild-type Targ polymerase can comprise the amino acid of SEQ ID NO: 31. In some embodiments, the engineered family B polymerase (e.g., engineered Targ) as described herein comprises one or more substitutions selected from the group consisting of an aspartic acid substitution at position 141; a glutamic acid substitution at position 143; an alanine substitution at position 488; a valine substitution at position 93; an arginine substitution at position 97; a tyrosine substitution at position 387; a valine substitution at position 392; a phenylalanine at position 496; a phenylalanine substitution at position 590; a glutamic acid substitution at position 667; a glycine substitution at position 714; a tryptophan substitution at position 771; an isoleucine substitution at position 2; an isoleucine substitution at position 38; a lysine substitution at position 118; a methionine substitution at position 137; an arginine substitution at position 384; a lysine substitution at position 469; a tyrosine substitution at position 517; an isoleucine substitution at position 524; and an asparagine substitution at position 738 of SEQ ID NO: 28.

In some embodiments, the engineered family B polymerase as described herein comprises one or more substitutions selected from the group consisting of an aspartic acid to alanine substitution at position 141 (D141A); a glutamic acid to alanine substitution at position 143 (E143A); an alanine to leucine substitution at position 488 (A488L); a valine to glutamine substitution at position 93 (V93Q); an arginine to methionine substitution at position 97 (R97M); a tyrosine to histidine substitution at position 387 (Y387H); a valine to isoleucine substitution at position 392 (V392I); a phenylalanine to leucine substitution at position 496 (F496L); a phenylalanine to leucine substitution at position 590 (F590L); a glutamic acid to lysine substitution at position 667 (E667K); a glycine to valine substitution at position 714 (G714V); a tryptophan to arginine substitution at position 771 (W771R); an isoleucine to valine substitution at position 2 (I2V); an isoleucine to leucine substitution at position 38 (I38L); a lysine to isoleucine substitution at position 118 (K118I); a methionine to leucine substitution at position 137 (M137L); an arginine to histidine substitution at position 384 (R384H); a lysine to arginine substitution at position 469 (K469R); a tyrosine to isoleucine substitution at position 517 (T517I); an isoleucine to leucine substitution at position 524 (I524L); and an asparagine to lysine substitution at position 738 (N738K) of SEQ ID NO: 28. In some embodiments, the engineered Targ comprises the amino acid sequence of SEQ ID NO: 28 or 29.

### 1. Engineered pfu

In some embodiments of the engineered family B polymerase described herein, the engineered family B polymerase is an engineered Pyrococcus furiosus (pfu) polymerase. The pfu may comprise the amino acid sequence of SEQ ID NO: 1. In some embodiments, the engineered family B polymerase described herein comprises an amino acid sequence having 90%, 91%, 92%, 93%, 94%, 95%, 96%, 97%, 98%, or 99% sequence identity to the amino acid sequence of SEQ ID NO: 1.

In some embodiments, the engineered family B polymerase comprises an amino acid substitution in SEQ ID NO: 1 selected from I38L, R97M, K118I, I137L, R382H, Y385H, V390I, K467R, F494L, T515I, I522L, F588L, E665K, S712V, N736K, or W769R in SEQ ID NO: 1, or any combination thereof, or the combination of all substitutions. The engineered family B polymerase can comprise I38L, R97M, K118I, I137L, R382H, Y385H, V390I, K467R, F494L, T515I, I522L, F588L, E665K, S712V, N736K, and W769R in SEQ ID NO: 1. In some embodiments, the engineered family B polymerase comprises any amino acid substitution selected from 38L, 97M, 118I, 137L, 382H, 385H, 390I, 467R, 494L, 515I, 522L, 588L, 665K, 712V, 736K, 769R, or any combination thereof, or the combination of all substitutions in SEQ ID NO: 1.

FIG. 6 shows that SEQ ID NO: 1 has one insertion at position 381 when compared to SEQ ID NO: 10 (FIG. 6B) and one insertion at position 773 (FIG. 6D).

In some embodiments, the engineered family B polymerase can comprise an amino acid substitution at any position in SEQ ID NO: 1 corresponding to position F38, R97, K118, M137, R381, Y384, V389I, K466R, Y493L, T514I, I521L, F587L, E664K, G711V, N735K, W768R in SEQ ID NO: 7.

In some embodiments, the engineered family B polymerase can further comprise an amino acid substitution at a position in SEQ ID NO: 1 corresponding to any one of position 12, V93, D141, E143, or A485 in SEQ ID NO: 7, or any combination thereof, or the combination of all substitutions. Alternatively, the substitutions can be I2V, V93Q, D141A, E143A, A485L, or any combination thereof, or the combination thereof in SEQ ID NO: 7. In some embodiments, the engineered family B polymerase further comprises one or more substitution selected from I2V, V93Q, D141A, E143A, or A486L in SEQ ID NO: 1. In some embodiments, the engineered family B polymerase further comprises I2V, V93Q, D141A, E143A, or A486L in SEQ ID NO: 1.

In some embodiments of the engineered family B polymerase described herein, the engineered family B polymerase is pfu and comprises the amino acid of SEQ ID NO: 1 and can further comprise one or more substitutions selected from the group consisting of: an aspartic acid substitution at position 141; a glutamic acid substitution at position 143; an alanine substitution at position 485; a valine substitution at position 93; an arginine substitution at position 97; a tyrosine substitution at position 384; a valine substitution at position 389; a phenylalanine at position 494; a phenylalanine substitution at position 588; a glutamic acid substitution at position 665; a serine substitution at position 712; a tryptophan substitution at position 769; an isoleucine substitution at position 2; an isoleucine substitution at position 38; a lysine substitution at position 118; a isoleucine substitution at position 137; an arginine substitution at position 381; a lysine substitution at position 466; a tyrosine substitution at position 514; an isoleucine substitution at position 521; and/an asparagine substitution at position 735 in SEQ ID NO: 1.

In some embodiments of the engineered family B polymerase described herein, the engineered family B polymerase is pfu and comprises the amino acid of SEQ ID NO: 1 and can further comprise one or more substitutions selected from the group consisting of: an aspartic acid to alanine substitution at position 141 (D141A); a glutamic acid to alanine substitution at position 143 (E143A); an alanine to leucine substitution at position 485 (A485L); a valine to glutamine substitution at position 93 (V93Q); an arginine to methionine substitution at position 97 (R97M); a tyrosine to histidine substitution at position 384 (Y384H); a valine to isoleucine substitution at position 389 (V389I); a phenylalanine to leucine substitution at position 494 (F494L); a phenylalanine to leucine substitution at position 588 (F588L); a glutamic acid to lysine substitution at position 665 (E665K); a serine to valine substitution at position 712 (S712V); a tryptophan to arginine substitution at position 769 (W769R); an isoleucine to valine substitution at position 2 (I2V); an isoleucine to leucine substitution at position 38 (I38L); a lysine to isoleucine substitution at position 118 (K118I); a isoleucine to leucine substitution at position 137 (I137L); an arginine to histidine substitution at position 381 (R381H); a lysine to arginine substitution at position 466 (K466R); a tyrosine to isoleucine substitution at position 514 (T514I); an isoleucine to leucine substitution at position 521 (I521L); and/an asparagine to lysine substitution at position 735 (N735K) in SEQ ID NO: 1.

In some embodiments, the engineered family B polymerase (pfu) comprises a substitution at positions 141 and/or 143 of SEQ ID NO: 1 and lacks proofreading activity. In some embodiments, the engineered family B polymerase (pfu) comprises a substitution at position 141 of SEQ ID NO: 1 and lacks proofreading activity.

In some embodiments, the engineered family B polymerase comprises R97M, D141A, E143A, Y385H, V393I, Y494L, F588L, E665K, S712V, and W769R substitutions in SEQ ID NO: 1. Alternatively, the engineered family B polymerase comprises I2V, I38L, R97M, K118I, I137L, E143A, R382H; Y385H, V390I, K465R, F494L, T515I, I522L, F588L, E665K, S712V, N736K, and W769R substitutions in SEQ ID NO: 1. The engineered family B polymerase can also comprise I2V, I38L, R97M, K118I, I137L, D141A, E143A, R382H, Y385H, V390I, K465R, F494L, T515I, I522L, F588L, E665K, S712V, N736K, and W769R substitutions in SEQ ID NO: 1. In some embodiments, the engineered family B polymerase comprises I2V, I38L, V93Q, R97M, K118I, I137L, D141A, E143A, R382H, Y385H, A486L, V390I, K467R, F494L, T515I, I522L, F588L, E665K, S712V, N736K, and W769R substitutions in SEQ ID NO: 1.

In some embodiments, the engineered pfu comprises the amino acid sequence of SEQ ID NO: 2, 3, 4, 5, 26, or 27. Alternatively, the engineered pfu can comprise an amino acid sequence having at least 72% identity to the amino acid sequence of SEQ ID NO: 2, 3, 4, 5, 26, or 27.

In one embodiment, the engineered family B polymerase, as described herein, comprises at least one, at least two, at least three, at least five, at least six, at least seven, at least eight, at least nine, at least ten, at least eleven, at least twelve, at least fifteen, or at least twenty of the substitutions disclosed herein in SEQ ID NO: 1, 10, 20-22, or 31. In some embodiments, the engineered family B polymerase described herein comprises at least about 10, at least about 15, at least about 16, at least about 18, at least about 20, at least about 25, or at least about 30 substitutions in the amino acid sequence of SEQ ID NO: 1. In some embodiments, the engineered family B polymerase described herein comprises at least 97% identity to the amino acid sequence of SEQ ID NO: 1, 10, 20-22, or 31 and at least about 16 substitutions in the amino acid sequence of SEQ ID NO: 1, 10, 20-22, or 31.

### 2. Engineered Tgo

In some embodiments of the engineered family B polymerase described herein, the engineered family B polymerase is an engineered Thermococcus gorgonarius polymerase (Tgo polymerase). In some embodiments, the wild-type Tgo comprises the amino acid of SEQ ID NO: 10.

In some embodiments, the engineered family B polymerase, as described herein, comprises one or more substitutions selected from the group consisting of an aspartic acid substitution at position 141; a glutamic acid substitution at position 143; an alanine substitution at position 485; a valine substitution at position 93; an arginine substitution at position 97; a tyrosine substitution at position 384; a valine substitution at position 389; a phenylalanine at position 493; a phenylalanine substitution at position 587; a glutamic acid substitution at position 664; a glycine substitution at position 711; a tryptophan substitution at position 768; an isoleucine substitution at position 2; an isoleucine substitution at position 38; a lysine substitution at position 118; a methionine substitution at position 137; an arginine substitution at position 381; a lysine substitution at position 466; a tyrosine substitution at position 514; an isoleucine substitution at position 521; and an asparagine substitution at position 735 of SEQ ID NO: 10. In some embodiments, the engineered Tgo enzyme comprises the amino acid sequence of SEQ ID NO: 11, 12, or 25.

In one embodiment, the engineered Tgo enzyme described herein comprises a combination of R97M, D141A, E143A, Y384H, V389I, Y493L, F587L, E664K, G711V, and W768R substitutions in SEQ ID NO: 10. In another embodiment, the engineered Tgo enzyme described herein comprises I2V, I38L, R97M, K118I, M137L, E143A, R381H; Y384H, V389I, K466R, F493L, T514I, I521L, F587L, E664K, G711V, N735K, and W768R substitutions in SEQ ID NO: 10. Yet in another embodiment, the engineered Tgo enzyme described herein comprises I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 10. Alternatively, the engineered Tgo enzyme described herein comprises I2V, I38L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 10.

In some embodiments of the engineered family B polymerase described herein, the engineered family B polymerase can bind a DNA, an RNA, or a DNA-RNA hybrid complex. The DNA-RNA hybrid can be continuous or discontinuous. The DNA-RNA hybrid can be a DNA structure in which one of the DNA stands is replaced with RNA. As used herein, “continuous DNA-RNA hybrid” refers to a DNA-RNA hybrid that does not contain a single strand break, which can be a nick (e.g., nicked DNA), or DNA-RNA hybrid that does not contain a DNA or RNA 3′- and 5′-overhangs. As used herein, “discontinuous DNA-RNA hybrid” refers to a DNA-RNA hybrid containing a single strand break, which can be incorporated with a nick (e.g., nick DNA), or DNA-RNA hybrid containing a DNA or RNA 3′- and 5′-overhangs. These terms have the same meaning as those used in the art.

Those of skilled in the art understand that nick and 3′-overhang structures are DNA replication intermediates. Indeed, during DNA replication, the overall growth of the antiparallel two daughter DNA chains appears to occur 5′-to-3′ direction in the leading-strand and 3′-to-5′ direction in the lagging-strand using enzyme system only able to elongate 5′-to-3′ direction. The lagging strand multistep synthesis reactions, called Discontinuous Replication Mechanism, involve short RNA primer synthesis, primer-dependent short DNA chains (Okazaki fragments) synthesis, primer removal from the Okazaki fragments and gap filling between Okazaki fragments by RNase H and DNA polymerase I, and long lagging strand formation by joining between Okazaki fragments with DNA ligase. See e.g., Okazaki T, Proc Jpn Acad Ser B Phys Biol Sci. 93(5): 322-338 (2017).

Accordingly, the ability to bind DNA-RNA hybrid complements can enhance the efficiency and processive characteristics of the engineered family B polymerase of the present disclosure. Indeed, endogenous polymerases possess at least three properties: (1) the 5′-to-3′ polymerase activity, (2) the 5′-to-3′ exonuclease activity, which is specific to double strand DNA or RNA-DNA hybrid molecules, and (3) the 3′-to-5′ exonuclease activity, which is specific to single-stranded DNA substrate and provides the proofreading function. When the 5′-to-3′ polymerase and the 5′-to-3′ exonuclease activities function in a coordinated manner, a nick on the double strand DNA migrates towards the 3′ direction and is eventually filled.

The engineered Tog-RTX enzyme of the present disclosure can comprise all these activities while also acting as a reverse transcriptase enzyme. Indeed, FIG. 3B shows that the enzyme disclosed herein can displaced about 6 nucleotides. This minimal strand-displacement activity can be attributed to the mutations introduced therein.

### B. Tag Proteins

One aspect of the present disclosure provides an engineered family B polymerase (e.g., a nucleic acid processing enzyme) comprising, consisting essentially of, or consisting of an amino acid sequence having at least 75% sequence identity to the amino acid sequence of Pyrococcus furiosus (pfu) polymerase (SEQ ID NO: 1), Thermococcus gorgonarius polymerase (Tgo polymerase) (SEQ ID NO: 10), Thermococcus litoralis (VENT®) polymerase (SEQ ID NO: 20), Pyrococcus sp. (Deep Vent) polymerase (SEQ ID NO: 21), Thermococcus sp. (9°N) polymerase (SEQ ID NO: 22), or Thermococcus argininiproducens (Targ) polymerase (SEQ ID NO: 31).

In some embodiments, the engineered family B polymerase described herein further comprises a tag protein selected from the group consisting of an affinity tag, a fluorescent tag, or an expression, and/or solubility enhancement tag. In some embodiments, the tag protein is selected from hexahistidine tag (his-tag), Fasciola hepatica 8-kDa antigen tag (Fh8), Glutathione-S-transferase (GST) tag, maltose-binding protein tag (MBP), Flag tag peptide (FLAG tag), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin), small ubiquitin-like modifier tag (SUMO), a strep tag, Thioredoxin (Trx) tag, a VariFlex™ C-Terminal solubility enhancement tag, a short peptide C-terminal tag, Solubility-enhancer peptide sequences (SET) tag, IgG domain B1 of Protein G (GB1) tag, IgG repeat domain ZZ of Protein A (ZZ) tag, Mutated dehalogenase tag (HaloTag), Solubility eNhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Stress-responsive proteins tag (e.g., RpoA, tag, SlyD Tsf tag, RpoS tag, PotD tag, or Crr tag), and E. coli acidic proteins tag (e.g., msyB tag, yigD tag, and rpoD tag). Additional affinity tags and solubility enhancer tags are known to those skill in the art. See Costa et al., Front. Microbiol., 63(5): (2014); Esposito and Chatterjee Curr. Opin. Biotechnol., 17: 353-358 (2006); Malhotra, A. “Tagging for protein expression,” in Guide to Protein Purification, 2nd Edn, eds. R. R. Burgess and M. P. Deutscher (San Diego, CA: Elsevier), 463:239-258 (2009).

In some embodiments, the tag is selected from hexahistidine tag (his-tag), small ubiquitin-like modifier tag (SUMO), a short peptide C-terminal tag, Thioredoxin (Trx) tag, a VariFlex™ C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin).

Tags used in the practice of the disclosure may serve any number of purposes and a number of tags may be added to impart one or more different functions to the engineered reverse transcriptase, and/or derivatives thereof, of the disclosure. For example, tags may (1) contribute to protein-protein interactions both internally within a protein and with other protein molecules, (2) make the protein amenable to particular purification methods, (3) enable one to identify whether the protein is present in a composition; or (4) give the protein other functional characteristics.

In one embodiment, the tag is an affinity tag selected from a histidine tag such as, a hexahistidine tag (his-tag or 6 His-tag), Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In one embodiment, the tag is a hexahistidine tag.

In some embodiments, the tag is selected from a small ubiquitin-like modifier tag (SUMO), a VariFlex™ C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the solubility enhancer tag is selected from the group consisting of a SUMO tag, a GST tag, a Trx tag, a VariFlex™ C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments, the tag is an affinity tag. In one embodiment, the tag is an affinity tag and comprises a histidine purification tag. In one embodiment, the tag is a hexahistidine tag (his tag). In one embodiment, the tag comprises an amino acid sequence of the sequence HHHHHH (SEQ ID NO: 13). In one embodiment, the tag is a solubility enhancer tag. In one embodiment, the solubility enhancer tag is a short peptide C-terminal tag. In one embodiment, the solubility enhancer tag comprises an amino acid sequence of SEEDEEKEEDG (SEQ ID NO: 14) or an amino acid sequence having at least 90% sequence identity to the amino acid sequence of SEQ ID NO: 14.

In some embodiments, the tag further comprises an endoprotein cleavage site selected from ENLYFQ/G (SEQ ID NO: 15), DDDDK/ (SEQ ID NO: 16), IEGR/ (SEQ ID NO: 18), LVPR/GS (SEQ ID NO: 148), or LEVLFQ/GP (SEQ ID NO: 19).

In some embodiments, the engineered family B polymerase or a derivative thereof further comprises a protease cleavage sequence. In some embodiments, the cleavage of the protease cleavage sequence by a protease results in cleavage of the affinity tag from the engineered reverse transcriptase enzyme or a derivative thereof. In some instances, the protease cleavage sequence/site is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase. In some embodiments, the protease cleavage sequence is a thrombin cleavage sequence.

In some embodiments, the tag is cleaved or removed from the engineered family B polymerase or derivatives thereof via the cleavage site. In one embodiment, the tag is cleaved or removed using an endoprotein selected from the group consisting of tobacco etch virus protease (Tev), enterokinase (EntK), factor Xa (Xa), thrombin (Thr), genetically engineered derivative of human rhinovirus 3C protease (PreScission), Catalytic core of Ulp1 (SUMO protease). In one embodiment, the tag is cleaved at ENLYFQ/G (SEQ ID NO: 15) using tobacco etch virus protease (Tev). In another embodiment, the tag is cleaved at DDDDK/ (SEQ ID NO: 16) using Enterokinase (EntK). In another embodiment, the tag is cleaved at IEGR/(SEQ ID NO: 17) using Factor Xa (Xa). In another embodiment, the tag is cleaved at LVPR/GS (SEQ ID NO: 18) using thrombin (Thr). In another embodiment, the tag is cleaved at LEVLFQ/GP (SEQ ID NO: 19) using a genetically engineered derivative of human rhinovirus 3C protease. In another embodiment, the tag is cleaved with Catalytic core of Ulp1 (SUMO protease). Catalytic core of Ulp1 recognizes SUMO tertiary structure and cleaves at the C-terminal end of the conserved Gly-Gly sequence in SUMO.

In some embodiments, the engineered family B polymerase or derivatives thereof comprises an affinity tag at the N-terminus or at the C-terminus of the amino acid sequence. In some embodiments, the affinity tag include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, the affinity tag is at least 5 histidine amino acids.

In some embodiments, the engineered family B polymerase comprises an amino acid sequence of SEQ ID NO: 11 or 12; or an amino acid sequence having at least 90% sequence identity to the amino acid sequence of SEQ ID NO: 11 or 12. In some embodiments, the engineered family B polymerase described herein or a derivative thereof comprises an amino acid sequence of ENLYFQ/G (SEQ ID NO: 11), DDDDK/ (SEQ ID NO: 12), IEGR/(SEQ ID NO: 13), LVPR/GS (SEQ ID NO: 14), or LEVLFQ/GP (SEQ ID NO: 15).

One of skill will recognize that modifications can additionally be made to the engineered family B polymerases (e.g., engineered family B polymerases; engineered DNA polymerase enzymes; engineered polymerases) of the present disclosure without diminishing their biological activity. Some modifications may be made to facilitate the cloning, expression, or incorporation of a domain into a fusion protein. Such modifications are well known to those of skill in the art and include, for example, the addition of codons at either terminus of the polynucleotide that encodes the binding domain to provide, for example, a methionine added at the amino terminus to provide an initiation site, or additional amino acids placed on either terminus to create conveniently located restriction sites or termination codons or purification sequences.

One or more of the domains of the engineered family B polymerases (e.g., engineered family B polymerases; engineered DNA polymerase enzymes; engineered polymerases) described herein may also be modified to facilitate the linkage of a variant enzyme described herein to obtain one or more polynucleotides that encode the engineered family B polymerases of the present disclosure. Thus, engineered family B polymerases that are modified by such methods are also part of the disclosure.

### C. Thermostability and Processivity

### 1. Thermostability

As used herein, the term “Thermostable” generally refers to an enzyme, such as a reverse transcriptase, or a polymerase, or an engineered family B polymerase (e.g., engineered family B polymerases; engineered DNA polymerase enzymes; engineered polymerases)), which retains a greater percentage or amount of its activity after a heat treatment than is retained by the same enzyme having wild type thermostability or a control enzyme having a certain thermostability, after an identical treatment. Thus, an r engineered family B polymerase having increased/enhanced thermostability may be defined as an engineered family B polymerase having any increase in thermostability, preferably from about 1.2 to about 10,000 fold, from about 1.5 to about 10,000 fold, from about 2 to about 5,000 fold, or from about 2 to about 2000 fold, or any value in between these amounts, and retention of activity after a heat treatment sufficient to cause a reduction in the activity of a reverse transcriptase that is wild type for thermostability or a control enzyme having a certain thermostability.

In other aspects of the disclosure, the increase in thermostability can be about 5 fold, about 10 fold, about 25 fold about 50 fold, about 75 fold, about 100 fold, about 150 fold, about 200 fold, about 300 fold, about 400 fold, about 500 fold, about 600 fold, about 700 fold, about 800, about 900 fold, or about 1000 fold.

In other aspects, the increase in thermostability is 1-5 fold, 5-10 fold, 10-15 fold, 15-20 fold, 20-25 fold, 25-30 fold, 30-35 fold, 35-40 fold, 40-45 fold, 45-50 fold, 50-55 fold, 55-60 fold, 60-65 fold, 65-70 fold, 70-75 fold, 75-80 fold, 80-85 fold, 85-90 fold, 90-95 fold, 95-100 fold, 100-105 fold, 105-110 fold, 110-115 fold, 115-120 fold, 120-125 fold, 125-130 fold, 135-135 fold, 135-140 fold, 140-145 fold, 145-150 fold, 150-200 fold, 200-250 fold, 250-300 fold, 300-350 fold.

In other aspects, the increase in thermostability is 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 31 fold, 32 fold, 33 fold, 34 fold, 35 fold, 36 fold, 37 fold, 38 fold, 39 fold, 40 fold, 42 fold, 44 fold, 46 fold, 48 fold, 50 fold, 52 fold, 54 fold, 56 fold, 58 fold, 60 fold, 62 fold, 64 fold, 68 fold, 70 fold, 72 fold, 74 fold, 76 fold, 78 fold, 80 fold, 82 fold, 84 fold, 86 fold, 88 fold, 90 fold, 92 fold, 94 fold, 96 fold, 98 fold, or 100 fold.

In other aspects, the increase in thermostability is 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.2 fold, 4.4 fold, 4.6 fold, 4.8 fold, 5.0 fold, 5.2 fold, 5.4 fold, 5.6 fold, 5.8 fold, 6.0 fold, 6.2 fold, 6.4 fold, 6.8 fold, 7.0 fold, 7.2 fold, 7.4 fold, 7.6 fold, 7.8 fold, 8.0 fold, 8.2 fold, 8.4 fold, 8.6 fold, 8.8 fold, 9.0 fold, 9.2 fold, 9.4 fold, 9.6 fold, 9.8 fold, or 10.0 fold.

To determine the thermostability of the engineered family B polymerase of the present disclosure, the engineered family B polymerase can be compared to the corresponding wild-type polymerase (e.g., Tgo, pfu, targ, or KOD1) and/or a wild type MMLV or a variant thereof (e.g., control) to determine the relative enhancement or increase in thermostability. In a non-limiting example, after a heat treatment at 60° C. for 5 minutes, the engineered family B polymerase may retain approximately 90% of the activity present before the heat treatment, whereas a wild type MMLV or a MMLV variant (e.g., FIGS. 2A-B) may retain 10% of its original activity. Likewise, after a heat treatment at 60° C. for 15 minutes, the engineered family B polymerase may retain approximately 80% of its original activity, whereas a wild type MMLV or a MMLV variant may have no measurable activity. Similarly, after a heat treatment at 60° C. for 15 minutes, the engineered family B polymerase may retain approximately 50%, approximately 55%, approximately 60%, approximately 65%, approximately 70%, approximately 75%, approximately 80%, approximately 85%, approximately 90%, or approximately 95% of its original activity, whereas a wild type MMLV or a MMLV variant may have no measurable activity or may retain 20%, 15%, 10%, or none of its original activity. In the first instance (i.e., after heat treatment at 60° C. for 5 minutes), the engineered family B polymerase would be said to be 9-fold more thermostable than the wild-type reverse transcriptase (90% compared to 10%). Examples of conditions which may be used to measure thermostability of an enzyme such as reverse transcriptases are set out in further detail below and in the Examples.

The thermostability of an engineered family B polymerase (e.g., engineered family B polymerases; engineered DNA polymerase enzymes; engineered polymerases) can be determined, for example, by comparing the residual activity of an engineered family B polymerase that has been subjected to a heat treatment, e.g., incubated at a certain temperature, e.g. without limitation 60° C. for a given period of time, for example, five minutes, to a control sample of the same reverse transcriptase that has been incubated at room temperature for the same length of time as the heat treatment. One way the residual activity may be measured is by following the incorporation of a radiolabeled deoxyribonucleotide into an oligodeoxyribonucleotide primer using a complementary oligoribonucleotide template. For example, the ability of the reverse transcriptase to incorporate [α-³²P]-dGTP into an oligo-dG primer using a poly(riboC) template may be assayed to determine the residual activity of the reverse transcriptase. Methods for measuring residual activity of reverse transcriptase and polymerases are known by those of skill in the art. See e.g., Nikiforov, T. T., Anal Biochem., 2011, 412(2): 229-36, which is hereby incorporated by reference.

In some embodiments, the engineered family B polymerase of the present disclosure is thermophilic. In one embodiment, the engineered family B polymerase is resistant to thermal inactivation when compared to a wild-type polymerase. In another embodiment, the engineered family B polymerase is resistant to thermal inactivation at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C. In yet another embodiment, the engineered family B polymerase is resistant to thermal inactivation at a temperature of about 68° C.

In certain embodiments, the engineered family B polymerases of the disclosure have high thermostability, e.g., thermostability at temperatures above 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

In certain embodiments, the engineered family B polymerases of the disclosure have high thermostability, e.g., thermostability at temperatures of 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

In certain embodiments, the engineered family B polymerases of the disclosure have high thermostability, e.g., thermostability at temperatures of about: 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

In another embodiment, the thermostability of the engineered family B polymerase is determined by measuring the half-life of the engineered family B polymerase. Such half-life may be compared to a control or wild type polymerase enzyme to determine the difference (or delta) in half-life.

### 2. Half-Life

In some embodiments, the engineered family B polymerase possesses an enhanced half-life when compared to a wild-type polymerase and/or a wild-type reverse transcriptase at a temperature from about 53° C. to about 75° C.; from about 55° C. to about 75° C.; from about 60° C. to about 75° C.; from about 53° C. to about 68° C.; from about 55° C. to about 68° C.; from about 45° C. to about 68° C.; or from about 50° C. to about 68° C.

In certain embodiments, half-life of the engineered family B polymerases of the disclosure is measured at temperatures above 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

In certain embodiments, half-life of the engineered family B polymerases of the disclosure is measured at temperatures of 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

In certain embodiments, half-life of the engineered family B polymerases of the disclosure is measured at temperatures of about: 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C. or more, and optionally have proofreading activity.

The half-life of the engineered family B polymerase of the disclosure is preferably determined at elevated temperatures (e.g., greater than 37° C.) and preferably at temperatures ranging from 40° C. to 80° C., or temperatures ranging from 45° C. to 75° C., 50° C. to 70° C., 55° C. to 65° C., and 58° C. to 62° C. Preferred half-lives of the engineered family B polymerase of the present disclosure may range from about 4 minutes to about 10 hours, about 4 minutes to about 7.5 hours, about 4 minutes to about 5 hours, about 4 minutes to about 2.5 hours, or about 4 minutes to about 2 hours, depending upon the temperature used. For example, the reverse transcriptase activity of the engineered family B polymerase of the present disclosure may have a half-life of at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 20 minute, at least about 25 minutes, at least about 30 minutes, at least about 40 minutes, at least about 50 minutes, at least about 60 minutes, at least about 70 minutes, at least about 80 minutes, at least about 90 minutes, at least about 100 minutes, at least about 115 minutes, at least about 125 minutes, at least about 150 minutes, at least about 175 minutes, at least about 200 minutes, at least about 225 minutes, at least about 250 minutes, at least about 275 minutes, at least about 300 minutes, at least about 400 minutes, at least about 500 minutes, or any time period in between these values, at temperatures of about 48° C., about 50° C., about 52° C., about 54° C., about 56° C., about 58° C., about 60° C., about 62° C., about 64° C., about 66° C., about 68° C., and/or about 70° C.

In some embodiments, the thermostability of the engineered family B polymerase enhances the half-life of the engineered family B polymerase.

In some embodiments, the engineered family B polymerase possesses one or more of the following characteristics when compared to a wild-type polymerase and/or a wild-type reverse transcriptase: increased thermostability; increased thermoreactivity; increased resistance to reverse transcriptase inhibitors; increased ability to reverse transcribe difficult templates; increased speed; increased processivity; increased specificity; enhanced polymerization activity; increased sensitivity, or any combination thereof.

### 3. Processivity

Processivity can be defined as the ability of a polymerase to carry out continuous nucleic acid synthesis on a template nucleic acid without frequent dissociation. It can be measured by the average number of nucleotides incorporated by a polymerase on a single association/disassociation event. DNA polymerase alone produces short DNA product strand per binding event. Most DNA polymerases are intrinsically low-processivity enzymes. The low processivity of DNA polymerase alone is insufficient for the timely replication of a large genome.

In some embodiments, the polymerization activity of the engineered family B polymerase as described herein is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments, the engineered family B polymerase reverse transcribes a RNA molecule having at least about 6, at least about 7, at least about 8, at least about 9, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 100, at least about 200, at least about 300, at least about 400, at least about 500, at least about 600, at least about 700, at least about 800, at least about 900, or at least about 1000 nucleotides.

In another embodiment, the engineered family B polymerase reverse transcribes a RNA molecule comprising 6, 7, 8, 9, 10, 15, 20, 25, 30, 35, 40, 45, 50, 55, 60, 65, 70, 75, 80, 85, at least about 90, 100, 200, 300, 400, 500, 600, 700, 800, 900, or 1000 nucleotides.

In some embodiments, the engineered family B polymerase reverse transcribes a RNA molecule that is at least about 1-1000, at least about 1-750, at least about 1-500, at least about 1-300, at least about 1-200, at least about 1-100, at least about 1-90, at least about 1-80, at least about 1-70, at least about 1-60, at least about 1-50, at least about 1-40, at least about 1-30, at least about 1-20, at least about 1-10, at least about 1-9, at least about 1-8, at least about 1-7, at least about 1-6, at least about 1-5, at least about 1-4, 1-3, or at least about 1-2 nucleotides. Alternatively, the engineered family B polymerase can reverse transcribe a RNA molecule that is 1-1000, 1-750, 1-500, 1-300, 1-200, 1-100, 1-90, 1-80, 1-70, at least about 1-60, 1-50, 1-40, 1-30, 1-20, 1-10, 1-9, 1-8, 1-7, 1-6, 1-5, 1-4, 1-3, or 1-2 nucleotides.

In another embodiment, the engineered family B polymerase reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In another embodiment, the engineered family B polymerase reverse transcribes a RNA molecule that is at least about 7 kb or at least about 8 kb.

In some embodiments, the increase in thermoreactivity, resistance to reverse transcriptase inhibitors, ability to reverse transcribe difficult templates, speed, processivity, specificity, or sensitivity of the engineered family B polymerase as described herein has is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

### 4. Strand Displacement

Synthetic Biology relies on the ability to build novel DNAs from component parts. Double strand (ds) DNA molecules have been assembled by creating staggered ends at the both ends of a first DNA duplex. This has been achieved using restriction endonucleases or by using exonuclease digestion or by a wild-type DNA polymerase (e.g., a T4 polymerase) followed by hybridization and optional ligation of a second DNA duplex to the first duplex.

In the RTL assay described herein, this characteristic is important to ensure that a hybridized oligonucleotide and/or probes are not removed by the polymerase during the extension of a first oligonucleotide. Without strand displacement, only the most 3′-directed primer to the preselected region is successfully extended to the location corresponding to the first primer.

As such, to generate a ligation product using exonucleases and ligases in a reaction mixture (e.g., RTL), a non-strand displacing polymerases or RT is preferred. An example of a non-strand displacing enzyme includes Phusion® polymerase (Thermo Fisher, Waltham, MA) (which is generally described as non-strand displacing), 9°N, Vent® or Pfu DNA polymerases. Additional DNA polymerases without strand displacement activity include T7, Q5 or T4 DNA polymerase. Indeed, as shown in FIGS. 3-4, non-strand displacing polymerases usually terminate synthesis of a template DNA when it encounters a blocking oligonucleotide.

Since DNA polymerases with strand displacement activity can displace a DNA oligonucleotide from a template strand of DNA at least as good as dissolving secondary or tertiary structure, the hybridization of the oligonucleotide and gap filling can be enhanced by using a non-strand displacing enzyme. In addition, the non-strand displacing requirement is necessary for successful post gap-fill ligation. Ligation typically does not occur if a portion of the probe is displaced, though a flap-endonuclease for example FEN1 endonuclease, could help remove the flap if some displacement occurs.

In the gap fill reactions and method described herein, an enzyme without strand displacing activity is desirable so as to fill in the gap between a first and a second probe which are not immediately adjacent to each other, without displacing the second/right hand side probe which may contain additional sequences which are not part of the nucleic acid target. Such additional sequences may include without limitation functional sequences such as constant sequence, probe barcode, and/or various capture sequences, or spatial capture sequences. These functional sequences are used in different steps of the methods of the disclosure. For non-limiting examples of functional sequences see User Guide CG000477, and the Visium Spatial Gene Expression Reagent Kits User Guide (e.g., Rev F, dated January 2022) cited infra.

### D. Nucleic Acids and Expression Vectors

### 1. Nucleic Acids

One aspect of the present disclosure provides an isolated nucleic acid molecule encoding the engineered family B polymerase or a derivatives thereof (e.g., engineered family B polymerases; engineered DNA polymerase enzymes; engineered polymerases) as described herein. In some embodiments, the engineered family B polymerase is encoded by a nucleic acid set forth herein or readily derived in light of polypeptide information provided herein and known in the art. The engineered family B polymerase described herein need not be encoded by any specific nucleic acid exemplified herein. For example, redundancy in the genetic code allows for variations in nucleotide codon sequences that nevertheless encode the same amino acid. Accordingly, engineered family B polymerases (i.e., polymerases) of the present disclosure can be produced from nucleic acid sequences that are different from those set forth herein, for example, being codon optimized for a particular expression system. Codon optimization can be carried out, for example, as set forth in Athey et al., BMC Bioinformatics, 18:391-401 (2017).

Wild type polymerase nucleic acids may be isolated from naturally occurring sources to be used as starting material to generate novel polymerases described herein. Generally, the nomenclature and the laboratory procedures in recombinant DNA technology described below are those well-known and commonly employed in the art. Standard techniques for cloning, DNA and RNA isolation, amplification and purification are known. Enzymatic reactions involving DNA ligase, DNA polymerase, restriction endonucleases are the like are performed according to the manufacturer's specifications. These techniques and various other techniques are generally performed according to Sambrook & Russell, Molecular Cloning—A Laboratory Manual, Cold Spring Harbor Laboratory, Cold Spring Harbor, N.Y., (1989) or Ausubel et al., Current Protocols in Molecular Biology, Vol. 1-3, John Wiley & Sons, Inc. (1994-1998).

The isolation of polymerase nucleic acids may be accomplished by a variety of techniques. The polymerase nucleic acids of the present disclosure can be generated from the wild type sequences. The wild type sequences can be altered to create modified sequences. Wild type polymerases (e.g., SEQ ID NO: 1, 10, 20, 21, 22, or 31 or variants thereof) can be modified to create the polymerases claimed in the present application using methods that are well known in the art. Exemplary modification methods are site-directed mutagenesis, point mismatch repair, or oligonucleotide-directed mutagenesis.

Methods of producing an engineered family B polymerase or a derivative thereof of the present disclosure are known to those of skill in the art of molecular biology or molecular genetics. For example, nucleic acids encoding the wild-type polymerase or nucleic acid binding domains can be generated using routine techniques in the field of recombinant genetics. Basic texts disclosing the general methods of use in this disclosure include Sambrook and Russell, Molecular Cloning, A Laboratory Manual (3rd ed. 2001); Kriegler, Gene Transfer and Expression: A Laboratory Manual (1990); Current Protocols in Molecular Biology (Ausubel et al., eds., 1994-1999); Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

### 2. Vectors

Another aspect of the present disclosure provides an expression vector comprising the isolated nucleic acid encoding the engineered family B polymerase or derivatives thereof as described herein. A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. The polymerases of the present disclosure can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsiella, Proteus, Salmonella, Serratia, Shigella, Rhizobia, Vitreoscilla, and Paracoccus. Filamentous fungi that are useful as expression hosts include, for example, the following genera: Aspergillus, Trichoderma, Neurospora, Penicillium, Cephalosporium, Achlya, Podospora, Mucor, Cochliobolus, and Pyricularia. See, e.g., U.S. Pat. No. 5,679,543 and Stahl and Tudzynski, Eds., Molecular Biology in Filamentous Fungi, John Wiley & Sons, 1992. Synthesis of heterologous proteins in yeast is well known and described in the literature. Methods in Yeast Genetics, Sherman F. et al., Cold Spring Harbor Laboratory (1982) is a well-recognized work describing the various methods available to produce the enzymes in yeast. There are many expression systems for producing the polymerase polypeptides of the present disclosure that are well known to those of ordinary skill in the art. See Gene Expression Systems, Fernandex and Hoeffler, Eds. Academic Press, 1999; Sambrook & Russell, supra; and Ausubel et al, Current Protocols in Molecular Biology, Vol. 1-3, John Wiley & Sons, Inc. (1994-1998).

### 3. Cells

Another aspect of the present disclosure provides a host cell transfected with the expression vector comprising the isolated nucleic acid encoding the engineered family B polymerase as described herein. Eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, rous sarcoma virus promoter, polyhedrin promoter, or other promoters shown effective for expression in eukaryotic cells.

Once expressed, the engineered family B polymerase or a derivative thereof can be purified according to standard procedures of the art, including ammonium sulfate precipitation, affinity purification columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification, Academic Press, Inc. N.Y. (1990)). Substantially pure compositions of at least about 90 to about 95% homogeneity are preferred, and about 98 to about 99% or more homogeneity are most preferred. Once purified, partially or to homogeneity as desired, the polypeptides may then be used (e.g., as immunogens for antibody production).

To facilitate purification of the engineered family B polymerase or a derivative thereof, the nucleic acids that encode the engineered family B polymerase or derivatives thereof can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the disclosure, and corresponding detection systems are known to those of skill in the art as described herein, and several are commercially available (e.g., FLAG (Kodak, Rochester N.Y.). Another example of a suitable tag is a polyhistidine sequence, which is capable of binding to metal chelate affinity ligands. Typically, six adjacent histidines are used (6His-tag, his-tag), although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

One of skill in the art would recognize that after biological expression or purification, the engineered family B polymerase or derivatives thereof may possess a conformation substantially different than the native conformations of the constituent polypeptides. In this case, it may be necessary or desirable to denature and reduce the engineered family B polymerase or a derivative thereof and cause the engineered family B polymerase or a derivative thereof to re-fold into the preferred conformation. Methods of reducing and denaturing proteins and inducing re-folding are well known to those of skill in the art (See Debinski et al. (1993) J. Biol. Chem., 268: 14065-14070; Kreitman and Pastan (1993) Bioconjug. Chem., 4: 581-585; and Buchner et al. (1992) Anal. Biochem., 205: 263-270). Debinski et al., for example, describe the denaturation and reduction of inclusion body proteins in guanidine-DTE. The protein is then refolded in a redox buffer containing oxidized glutathione and L-arginine.

### E. Compositions and Reaction Mixtures Comprising the Engineered Family B Polymerase or Derivatives Thereof

The present disclosure further provides compositions comprising a variety of components in various combinations needed for nucleic acid amplification. In some embodiments of the present disclosure, the compositions are formulated by admixing one or more engineered family B polymerases or derivatives thereof (e.g., engineered family B polymerases; engineered DNA polymerase enzymes; engineered polymerases) of the present disclosure in a buffered salt solution. One or more DNA polymerases and/or one or more nucleotides, and/or one or more primers may optionally be added to create the compositions of the disclosure. These compositions can be used in the methods disclosed herein to produce, analyze, quantitate and otherwise manipulate nucleic acid molecules (e.g., using reverse transcription or one-step RT-PCR procedures).

In some embodiments, the engineered family B polymerases are provided at working concentrations (e.g., 1×) in stable buffered salt solutions. The terms “stable” and “stability” as used herein generally mean the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4° C., about two to six months at a temperature of about −20° C., and about six months or longer at a temperature of about −80° C. As used herein, the term “working concentration” means the concentration of an enzyme that is at or near the optimal concentration used in a solution to perform a particular function such as reverse transcription of nucleic acids.

Such compositions can also be formulated as concentrated stock solutions (e.g., 2×, 3×, 4×, 5×, 6×, 10×, etc.). In some embodiments, having the composition as a concentrated (e.g., 5×) stock solution allows a greater amount of nucleic acid sample to be added (such as, for example, when the compositions are used for nucleic acid synthesis). The water used in forming the compositions of the present disclosure is preferably distilled, deionized and sterile filtered (through a 0.1-0.2 micrometer filter),and is free of contamination by DNase and RNase enzymes. Such water is available commercially, for example from Life Technologies (Carlsbad, Calif.) or may be made as needed according to methods well known to those skilled in the art.

### III. Methods of Using the Engineered Enzyme or a Derivative Thereof

### A. Amplification Methods

Another aspect of the present disclosure provides a method of using an engineered family B polymerase or a derivative thereof as described herein, the method comprising, consisting essentially of, or consisting of contacting the engineered family B polymerase or a derivative thereof (e.g., engineered family B polymerases; engineered DNA polymerase enzymes; engineered polymerases) with a with a plurality of nucleic acid templates under suitable conditions to produce a polymerized nucleic acid product.

The engineered family B polymerase can comprise an amino acid sequence having at least 75% sequence identity to the amino acid sequence of Pyrococcus furiosus (pfu) polymerase (SEQ ID NO: 1). The engineered family B polymerase can comprise an amino acid sequence having at least 75% sequence identity to the amino acid sequence of Thermococcus gorgonarius polymerase (Tgo polymerase) (SEQ ID NO: 10). The engineered family B polymerase can comprise an amino acid sequence having at least 75% sequence identity to the amino acid sequence of Thermococcus kodakarensis (KOD1) polymerase (SEQ ID NO: 6 or 8). The engineered family B polymerase can comprise an amino acid sequence having at least 75% sequence identity to the amino acid sequence of Thermococcus litoralis (VENT®) polymerase (SEQ ID NO: 20). The engineered family B polymerase can comprise an amino acid sequence having at least 75% sequence identity to the amino acid sequence of Pyrococcus sp. (Deep Vent)polymerase (SEQ ID NO: 21). The engineered family B polymerase can comprise an amino acid sequence having at least 75% sequence identity to the amino acid sequence of Thermococcus sp. (9°N) polymerase (SEQ ID NO: 22). The engineered family B polymerase can comprise an amino acid sequence having at least 75% sequence identity to the amino acid sequence of Thermococcus argininiproducens (Targ) polymerase (SEQ ID NO: 31).

The engineered family B polymerase is an engineered polymerase enzyme that has reverse transcriptase activity and substantially lacks strand displacement activity. Optionally, the engineered family B polymerase has no detectable strand displacement activity. In some embodiments, the engineered family B polymerase has no detectable strand displacement activity when it cannot amplify at least 1 nucleotide of the template in the presence of a blocking oligo and does not produce the full-length expected product or any intermediate products. For example, as shown in FIG. 4B, KOD-RTX showed no strand displacement activity. In fact, the amplified product appeared before the expected start of the blocking oligo.

In some embodiments, the engineered family B polymerase has minimal strand displacement activity when the engineered family B polymerase can amplify a template in the presence of a blocking oligo but does not generate the expected full-length product. (e.g., FIG. 3B). The engineered family B polymerase can displace no more than 10, 9, 8, 7, 6, 5, 4, 3, 2, or 1 nucleotides. In some embodiments, the engineered family B polymerase can displace 1-2, 1-3, 1-4, 1-5, 1-6, 1-7, 1-8, 1-9, or 1-10 nucleotides. In some embodiments, the engineered family B polymerase can displace 1, 2, 3, 4, 5, 6, 7, 8, 9 or 10 nucleotides. In some embodiments, the engineered family B polymerase can displace about 6 nucleotides. In some embodiments, the engineered family B polymerase can displace about 10 nucleotides.

In some embodiments, the nucleic acid template comprises a first probe and a second probe, which are hybridized to a first and a second target nucleic acids/target regions. Optionally, the second target nucleic acid/target region can be a mRNA. The first probe can be operably linked to the second probe. Alternatively, the first probe and the second probe can be part of the same molecule. The first probe and the second probe can be part of different molecules. In some embodiments, the polymerized product can be generated between the first probe hybridized to the first target sequence/target region and the second probe hybridized to the second target sequence/target region.

In some embodiments, the first probe hybridized to the first target sequence and the second probe hybridized to the second target sequence are not immediately adjacent to each other. Optionally, there is more than 1 nucleotide between the first and the second target sequences and/or the first and the second probes.

In some embodiments, the polymerized product can be generated between the first probe hybridized to the first target sequence/target region and the second probe hybridized to the second target sequence/target region.

In some embodiments, the first and the second target sequences or the first and the second probes hybridized to the first and the second target sequences are separated. In some embodiments, the first and the second target sequences or the first and the second probes hybridized to the first and the second target sequences are separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides.

In some embodiments, the first and the second target sequences or the first and the second probes hybridized to the first and the second target sequences are separated by 1-1000, 1-750, 1-500, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides.

The plurality of nucleic acid templates can be located in a biological sample. The biological sample can comprise a Formalin-Fixed Paraffin-Embedded (FFPE) sample, a formalin-fixed sample, a paraffin-embedded sample, a frozen sample, or a fresh sample. The biological sample can comprise a single cell. The biological sample can comprise a tissue.

In some embodiments of the method described herein, the method can determine the presence of a genetic variant in a nucleic acid. Optionally, the variant is at a spatial location in the biological sample. In some embodiments, the method can determine the location of a genetic variant in a target nucleic acid in the biological sample. In some embodiments, the method can comprise RNA-templated ligation.

In some embodiments, the plurality of nucleic acid templates can be a plurality of RNAs, DNAs, or nucleic acids comprising an unnatural nucleotide.

The engineered family B polymerase or a derivative thereof as described herein may be used to make nucleic acid molecules from one or more templates. Such methods can comprise mixing one or more nucleic acid templates (e.g., DNA or RNA, such as non-coding RNA (ncRNA), messenger RNA (mRNA), micro RNA (miRNA), and small interfering RNA (siRNA) molecules) with one or more of the reverse transcriptases of the disclosure and incubating the mixture under conditions sufficient to generate one or more nucleic acid molecules complementary to all or a portion of the one or more nucleic acid templates. Other methods of cDNA synthesis which may advantageously use the present disclosure will be readily apparent to one of ordinary skill in the art.

In some embodiments, the method of using the engineered family B polymerase or a derivative thereof as described herein (e.g., engineered family B polymerases; engineered DNA polymerase enzymes; engineered polymerases) can comprise the amplification of one or more nucleic acid molecules comprising mixing one or more nucleic acid templates with one of the engineered family B polymerases or derivative thereof of the disclosure. The mixture can be incubated under conditions sufficient to amplify the one or more nucleic acid molecules complementary to all or a portion of the one or more nucleic acid templates. In one embodiment, the method may further comprise the use of one or more DNA polymerases and may be employed as in standard reverse transcription-polymerase chain reaction (RT-PCR) reactions. In another embodiment, the method can only comprise an engineered family B polymerase or a derivative thereof (e.g., Tgo enzyme) that functions in a single-step reverse transcription-polymerase chain reaction.

In some embodiments, the method of using the engineered family B polymerase or a derivative thereof as described herein may be one-step (e.g., one-step RT-PCR) or two-step (e.g., two-step RT-PCR) reactions. In one embodiment, the one-step RT-PCR type reactions may be accomplished in one tube thereby lowering the possibility of contamination. Such one-step reactions comprise (a) mixing a nucleic acid template (e.g., mRNA) with one or more engineered family B polymerases or derivatives thereof of the present disclosure (e.g., Tgo enzyme) and (b) incubating the mixture under conditions sufficient to amplify a nucleic acid molecule complementary to all or a portion of the template. Such amplification may be accomplished by the reverse transcriptase activity of the engineered family B polymerase alone (e.g., Tgo enzyme) or in combination with the DNA polymerase activity of the engineered family B polymerase.

In another embodiment, a two-step RT-PCR reaction may be accomplished in two separate steps. Such a method comprises (a) mixing a nucleic acid template (e.g., mRNA) with an engineered family B polymerase or a derivative thereof of the present disclosure, (b) incubating the mixture under conditions sufficient to make a nucleic acid molecule (e.g., a DNA molecule) complementary to all or a portion of the template, (c) mixing the nucleic acid molecule with one or more DNA polymerases and (d) incubating the mixture of step (c) under conditions sufficient to amplify the nucleic acid molecule. For amplification of long nucleic acid molecules (i.e., greater than about 3-5 kb in length), a combination of DNA polymerases and the engineered family B polymerase or a derivative thereof of the present disclosure may be used.

Amplification methods which may be used with one or more engineered family B polymerases or derivatives thereof of the present disclosure can include PCR, Isothermal Amplification, Strand Displacement Amplification (SDA), Reverse Transcription Loop-mediated Isothermal Amplification (RT-Lamp), self-sustained sequence replication reaction (3SR), transcription mediated amplification (TMA), Rolling circle amplification (RCA), Recombinase polymerase amplification (RPA), or helicase-dependent amplification (HAD), and Nucleic Acid Sequence-Based Amplification (NASBA); as well as more complex PCR-based nucleic acid fingerprinting techniques such as Random Amplified Polymorphic DNA (RAPD) analysis, Arbitrarily Primed PCR (AP-PCR) DNA Amplification Fingerprinting (DAF); microsatellite PCR; Directed Amplification of Minisatellite-region DNA (DAVID); digital droplet PCT (ddPCR) and Amplification Fragment Length Polymorphism (AFLP) analysis. See, e.g., EP 0 534 858; Vos, P., et al. Nucl. Acids Res. 23(21):4407-4414 (1995); Lin, J. J., and Kuo, J. FOCUS 17(2):66-70 (1995); U.S. Pat. Nos. 4,683,195 and 4,683,202; PCT Publication No. WO 2006/081222; U.S. Pat. No. 5,455,166; EP 0 684 315. U.S. Pat. No. 5,409,818; EP 0 329 822; Williams, J. G. K., et al., Nucl. Acids Res. 18(22):6531-6535, (1990); Welsh, J., and McClelland, M., Nucl. Acids Res. 18(24):7213-7218 (1990); Caetano-Anollés et al., Bio/Technology 9:553-557 (1991); Heath, D. D., et al. Nucl. Acids Res. 21(24): 5782-5785 (1993). Nucleic acid sequencing techniques which may employ the present compositions include dideoxy sequencing methods such as those disclosed in U.S. Pat. Nos. 4,962,022 and 5,498,523.

In some embodiments, the engineered family B polymerases (e.g., engineered family B polymerases; engineered DNA polymerase enzymes; engineered polymerases) may be used in methods of amplifying or sequencing a nucleic acid molecule comprising one or more polymerase chain reactions (PCRs), such as any of the PCR-based methods described above.

In some embodiments, the method determines the presence of a genetic variant in a nucleic acid at a spatial location in the biological sample. In some embodiments, the method determines the location of a target nucleic acid in the biological sample. In some embodiments, the method can comprise RNA-templated ligation.

### B. Nucleic Acid Sample Processing

One aspect of the present disclosure provides a nucleic acid extension method comprising contacting a target nucleic acid molecule with an engineered family B polymerase or a derivative thereof (e.g., engineered family B polymerases; engineered DNA polymerase enzymes; engineered polymerases) and a plurality of nucleic acid barcoded molecules comprising a barcode sequence (e.g., a capture probe), and incubating the target nucleic acid, the engineered family B polymerase or a derivative thereof and barcoded molecules under conditions in which the barcoded molecules are extended by the engineered family B polymerase. The target nucleic acid hybridizes to one of the plurality of barcoded molecules and the hybridized barcoded molecule is extended by the engineered family B polymerase using the target nucleic acid (e.g., RNA, mRNA) as a template, thereby creating a first strand nucleic acid (e.g., cDNA).

In some embodiments, the engineered family B polymerase comprises an amino acid sequence having at least 75% sequence identity to the amino acid sequence of Pyrococcus furiosus (pfu) polymerase (SEQ ID NO: 1). The engineered family B polymerase can comprise an amino acid sequence having at least 75% sequence identity to the amino acid sequence of Thermococcus gorgonarius polymerase (Tgo polymerase) (SEQ ID NO: 10). The engineered family B polymerase can comprise an amino acid sequence having at least 75% sequence identity to the amino acid sequence of Thermococcus litoralis (VENT®) polymerase (SEQ ID NO: 20). The engineered family B polymerase can comprise an amino acid sequence having at least 75% sequence identity to the amino acid sequence of Pyrococcus sp. (Deep Vent) polymerase (SEQ ID NO: 21). The engineered family B polymerase can comprise an amino acid sequence having at least 75% sequence identity to the amino acid sequence of Thermococcus sp. (9°N) polymerase (SEQ ID NO: 22). The engineered family B polymerase can comprise an amino acid sequence having at least 75% sequence identity to the amino acid sequence of Thermococcus argininiproducens (Targ) polymerase (SEQ ID NO: 31).

In some embodiments, the engineered family B polymerase has reverse transcriptase activity and substantially lacks strand displacement activity as described herein. Alternatively, the engineered family B polymerase has reverse transcriptase activity and has no detectable strand displacement activity.

In some embodiments, the engineered family B polymerase can comprise an amino acid sequence that has at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31.

In some embodiments, the engineered family B polymerase described herein comprises 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 6, 8, 10, 20-22, or 31. In some embodiments, the engineered family B polymerase described herein comprises 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 10, 11, or 12. In some embodiments, the engineered family B polymerase described herein comprises the amino acid sequence of SEQ ID NO: 10, 11, or 12.

In some embodiments, the engineered family B polymerase can also have at least 95% identity to the amino acid sequence of SEQ ID NO: 1, 10, 20-22, or 31. Alternatively, the engineered family B polymerase has at least 97% identity to the amino acid sequence of SEQ ID NO: 1, 10, 20-22, or 31.

In some embodiments, the engineered family B polymerase can comprise a substitution at a position corresponding to a position selected from 38, 97, 118, 137, 381, 384, 389I, 466, 493, 514, 521, 587, 664, 711, 735, or 768 in SEQ ID NO: 7, or any combination thereof, or the combination of all substitutions. Alternative, the enzyme can comprise an amino acid substitution at positions corresponding to a position selected from selected from 38, 97, 118, 137, 381, 384, 389L, 466, 493, 514, 521, 587, 664, 711, 735, and 768 in SEQ ID NO: 6.

In some embodiments, the engineered family B polymerase comprises a substitution corresponding to any one amino acid substitution selected from 38L, 97M, 118I, 137L, 381H, 384H, 389I, 466R, 493L, 514I, 521L, 587L, 664K, 711V, 735K, 768R, or any combination thereof, or the combination of all substitutions. FIGS. 6A-D show relevant corresponding positions for contemplated substitution. An identity matrix showing the sequence homology/identity between the sequences is shown in FIG. 7

In some embodiments of the engineered family B polymerase described herein, the engineered family B polymerase further comprises an amino acid substitution at any position corresponding to position I2, V93, D141, E143, A485 in SEQ ID NO: 7, or any combination thereof, or the combination of all substitutions. Optionally, in some embodiments, the substitutions are I2V, V93Q, D141A, E143A, A485L, or any combination thereof, or the combination thereof in SEQ ID NO: 7. In those embodiments, the engineered family B polymerase comprises the amino acid sequence of SEQ ID NO: 11, 12, 25, or 28-30.

In some embodiments, the engineered family B polymerase as described herein comprises one or more substitutions selected from the group consisting of an aspartic acid substitution at position 141; a glutamic acid substitution at position 143; an alanine substitution at position 485; a valine substitution at position 93; an arginine substitution at position 97; a tyrosine substitution at position 384; a valine substitution at position 389; a phenylalanine at position 493; a phenylalanine substitution at position 587; a glutamic acid substitution at position 664; a glycine substitution at position 711; a tryptophan substitution at position 768; an isoleucine substitution at position 2; an isoleucine substitution at position 38 (I38L); a lysine substitution at position 118 (K118I); a methionine to leucine substitution at position 137 (M137L); an arginine to histidine substitution at position 381 (R381H); a lysine to arginine substitution at position 466 (K466R); a tyrosine to isoleucine substitution at position 514 (T514I); an isoleucine to leucine substitution at position 521 (I521L); and an asparagine to lysine substitution at position 735 (N735K) of SEQ ID NO: 10.

In some embodiments, the engineered family B polymerase as described herein comprises one or more substitutions selected from the group consisting of an aspartic acid to alanine substitution at position 141 (D141A); a glutamic acid to alanine substitution at position 143 (E143A); an alanine to leucine substitution at position 485 (A485L); a valine to glutamine substitution at position 93 (V93Q); an arginine to methionine substitution at position 97 (R97M); a tyrosine to histidine substitution at position 384 (Y384H); a valine to isoleucine substitution at position 389 (V389I); a phenylalanine to leucine substitution at position 493 (F493L); a phenylalanine to leucine substitution at position 587 (F587L); a glutamic acid to lysine substitution at position 664 (E664K); a glycine to valine substitution at position 711 (G711V); a tryptophan to arginine substitution at position 768 (W768R); an isoleucine to valine substitution at position 2 (I2V); an isoleucine to leucine substitution at position 38 (I38L); a lysine to isoleucine substitution at position 118 (K118I); a methionine to leucine substitution at position 137 (M137L); an arginine to histidine substitution at position 381 (R381H); a lysine to arginine substitution at position 466 (K466R); a tyrosine to isoleucine substitution at position 514 (T514I); an isoleucine to leucine substitution at position 521 (I521L); and an asparagine to lysine substitution at position 735 (N735K) of SEQ ID NO: 10.

In some embodiments of the engineered family B polymerase described herein, the engineered family B polymerase is an engineered Thermococcus kodakarensis (KOD1). In some embodiments, the wild-type KOD polymerase comprises the amino acid of SEQ ID NO: 6 or 8. In some embodiments, the engineered KOD1 comprises the amino acid sequence of SEQ ID NO: 7, 9, or 30.

In some embodiments of the engineered family B polymerase described herein, the engineered family B polymerase is an engineered Thermococcus argininiproducens (Targ) polymerase. In some embodiments, the wild-type Targ polymerase can comprise the amino acid of SEQ ID NO: 31. In some embodiments, the engineered KOD1 comprises the amino acid sequence of SEQ ID NO: 28 or 29.

### 1. Engineered pfu

In some embodiments of the engineered family B polymerase described herein, the engineered family B polymerase is an engineered Pyrococcus furiosus (pfu) polymerase. The pfu may comprise the amino acid sequence of SEQ ID NO: 1. In some embodiments, the engineered family B polymerase described herein comprises an amino acid sequence having 90%, 91%, 92%, 93%, 94%, 95%, 96%, 97%, 98%, or 99% sequence identity to the amino acid sequence of SEQ ID NO: 1.

In some embodiments, the engineered family B polymerase comprises an amino acid substitution in SEQ ID NO: 1 selected from I38L, R97M, K118I, I137L, R382H, Y385H, V390I, K467R, F494L, T515I, I522L, F588L, E665K, S712V, N736K, or W769R in SEQ ID NO: 1, or any combination thereof, or the combination of all substitutions. The engineered family B polymerase can comprise I38L, R97M, K118I, I137L, R382H, Y385H, V390I, K467R, F494L, T515I, I522L, F588L, E665K, S712V, N736K, and W769R in SEQ ID NO: 1. In some embodiments, the engineered family B polymerase comprises any amino acid substitution selected from 38L, 97M, 118I, 137L, 382H, 385H, 390I, 467R, 494L, 515I, 522L, 588L, 665K, 712V, 736K, 769R, or any combination thereof, or the combination of all substitutions in SEQ ID NO: 1.

In some embodiments, the engineered family B polymerase can comprise an amino acid substitution at any position in SEQ ID NO: 1 corresponding to position F38, R97, K118, M137, R381, Y384, V389L, K466R, Y493L, T514I, I521L, F587L, E664K, G711V, N735K, W768R in SEQ ID NO: 7.

In some embodiments, the engineered family B polymerase can further comprise an amino acid substitution at a position in SEQ ID NO: 1 corresponding to any one of position I2, V93, D141, E143, or A485 in SEQ ID NO: 7, or any combination thereof, or the combination of all substitutions. Alternatively, the substitutions can be I2V, V93Q, D141A, E143A, A485L, or any combination thereof, or the combination thereof in SEQ ID NO: 7. In some embodiments, the engineered family B polymerase further comprises one or more substitution selected from I2V, V93Q, D141A, E143A, or A486L in SEQ ID NO: 1. In some embodiments, the engineered family B polymerase further comprises I2V, V93Q, D141A, E143A, or A486L in SEQ ID NO: 1.

In some embodiments of the engineered family B polymerase described herein, the engineered family B polymerase is pfu and comprises the amino acid of SEQ ID NO: 1 and can further comprise one or more substitutions selected from the group consisting of: an aspartic acid substitution at position 141; a glutamic acid substitution at position 143; an alanine substitution at position 485; a valine substitution at position 93; an arginine substitution at position 97; a tyrosine substitution at position 384; a valine substitution at position 389; a phenylalanine substitution at position 494; a phenylalanine substitution at position 588; a glutamic acid substitution at position 665; a serine substitution at position 712; a tryptophan substitution at position 769; an isoleucine substitution at position 2; an isoleucine substitution at position 38; a lysine substitution at position 118; a isoleucine substitution at position 137; an arginine substitution at position 381; a lysine substitution at position 466; a tyrosine substitution at position 514; an isoleucine substitution at position 521; and/an asparagine substitution at position 735 in SEQ ID NO: 1.

In some embodiments of the engineered family B polymerase described herein, the engineered family B polymerase is pfu and comprises the amino acid of SEQ ID NO: 1 and can further comprise one or more substitutions selected from the group consisting of: an aspartic acid to alanine substitution at position 141 (D141A); a glutamic acid to alanine substitution at position 143 (E143A); an alanine to leucine substitution at position 485 (A485L); a valine to glutamine substitution at position 93 (V93Q); an arginine to methionine substitution at position 97 (R97M); a tyrosine to histidine substitution at position 384 (Y384H); a valine to isoleucine substitution at position 389 (V389I); a phenylalanine to leucine substitution at position 494 (F494L); a phenylalanine to leucine substitution at position 588 (F588L); a glutamic acid to lysine substitution at position 665 (E665K); a serine to valine substitution at position 712 (S712V); a tryptophan to arginine substitution at position 769 (W769R); an isoleucine to valine substitution at position 2 (I2V); an isoleucine to leucine substitution at position 38 (I38L); a lysine to isoleucine substitution at position 118 (K118I); a isoleucine to leucine substitution at position 137 (I137L); an arginine to histidine substitution at position 381 (R381H); a lysine to arginine substitution at position 466 (K466R); a tyrosine to isoleucine substitution at position 514 (T514I); an isoleucine to leucine substitution at position 521 (I521L); and/an asparagine to lysine substitution at position 735 (N735K) in SEQ ID NO: 1.

In some embodiments, the engineered family B polymerase (pfu) comprises a substitution at positions 141 and 143 of SEQ ID NO: 1 and lacks proofreading activity. In some embodiments, the engineered family B polymerase (pfu) comprises a substitution at position 141 of SEQ ID NO: 1 and lacks proofreading activity.

In some embodiments, the engineered family B polymerase comprises R97M, D141A, E143A, Y385H, V393I, Y494L, F588L, E665K, S712V, and W769R substitutions in SEQ ID NO: 1. Alternatively, the engineered family B polymerase comprises I2V, I38L, R97M, K118I, I137L, E143A, R382H; Y385H, V390I, K465R, F494L, T515I, I522L, F588L, E665K, S712V, N736K, and W769R substitutions in SEQ ID NO: 1. The engineered family B polymerase can also comprise I2V, I38L, R97M, K118I, I137L, D141A, E143A, R382H, Y385H, V390I, K465R, F494L, T515I, I522L, F588L, E665K, S712V, N736K, and W769R substitutions in SEQ ID NO: 1. In some embodiments, the engineered family B polymerase comprises I2V, I38L, V93Q, R97M, K118I, I137L, D141A, E143A, R382H, Y385H, A486L, V390I, K467R, F494L, T515I, I522L, F588L, E665K, S712V, N736K, and W769R substitutions in SEQ ID NO: 1.

In some embodiments, the engineered pfu comprises the amino acid sequence of SEQ ID NO: 2, 3, 4, 5, 26, or 27. Alternatively, the engineered pfu can comprise an amino acid sequence having at least 72% sequence to SEQ ID NO: 2, 3, 4, 5, 26, or 27.

### 2. Engineered Tgo

In some embodiments of the engineered family B polymerase described herein, the engineered family B polymerase is an engineered Thermococcus gorgonarius polymerase (Tgo polymerase). In some embodiments, the wild-type Tgo comprises the amino acid of SEQ ID NO: 10. In some embodiments, the engineered Tgo comprises the amino acid sequence of SEQ ID NO: 11, 12, or 25.

In one embodiment, the engineered Tgo enzyme described herein comprises a combination of R97M, D141A, E143A, Y384H, V389I, Y493L, F587L, E664K, G711V, and W768R substitutions in SEQ ID NO: 10. In another embodiment, the polymerase domain of the engineered Tgo enzyme described herein comprises I2V, I38L, R97M, K118I, M137L, E143A, R381H; Y384H, V389I, K466R, F493L, T514I, I521L, F587L, E664K, G711V, N735K, and W768R substitutions in SEQ ID NO: 10. Yet in another embodiment, the engineered Tgo enzyme described herein comprises I2V, I38L, R97M, K118I, M137L, D141A, E143A, R381H, Y384H, V389I, K466R, F493L, T514I, I521L, F587L, E664K, G711V, N735K, and W768R substitutions in SEQ ID NO: 10. Alternatively, the engineered Tgo enzyme described herein comprises I2V, I38L, V93Q, R97M, K118I, M137L, D141A, E143A, R381H, Y384H, A485L, V389I, K466R, F493L, T514I, I521L, F587L, E664K, G711V, N735K, and W768R substitutions in SEQ ID NO: 10.

In some embodiments of the nucleic acid extension method disclosed herein, the engineered family B polymerase comprises any of the amino acid sequence disclosed herein. In some embodiments, the engineered family B polymerase comprises the amino acid sequence set forth in SEQ ID NO: 2-5, 11, 12, and 25-30 or an amino acid sequence having at least 90% sequence identity to the amino acid sequence set forth in SEQ ID NO: 2-5, 11, 12, and 25-30.

In some embodiments, the one of the plurality of nucleic acid barcoded molecules hybridizes to the target nucleic acid molecule; and the engineered family B polymerase extends the one of the plurality of nucleic acid barcoded molecules that is hybridized to the target nucleic acid molecule.

### 3. RNA Template

In some embodiments, the nucleic acid is a ribonucleic acid (RNA) molecule; and the engineered family B polymerase reverse transcribes the RNA molecule thereby generating a first strand cDNA, and subsequently or concurrently amplifies the cDNA into a nucleic acid product in the same reaction. In one embodiment, the RNA molecule is a messenger RNA (mRNA) molecule.

In some embodiments of the nucleic acid extension method as described herein, each of the plurality of nucleic acid barcoded molecules comprises a molecular tag. Molecular tags include unique molecular identifiers (UMIs) and the UMIs comprise a polynucleotide. In some embodiments, the nucleic acid barcoded molecules further comprise capture sequences. A capture sequence can comprise a random N-mer sequence where the random N-mer sequence is complementary to a 3′ sequence of the RNA molecules. In some embodiments, the capture sequence comprises a poly-dT sequence having a length of at least 5 bases. In some embodiments, the capture sequence comprises a poly-dT sequence having a length of at least 10 bases. In some embodiments, the capture sequence comprises a poly-dT sequence having a length of at least 5 bases, at least 6 bases, at least 7 bases, at least 8 bases, at least 9 bases, at least 10 bases.

In some embodiments, a reverse transcription reaction of the engineered family B polymerase of the present disclosure is initiated at the point of hybridization of the capture sequences to the RNA molecules, with the capture probe being extended by the engineered family B polymerase of the present disclosure in a template directed fashion using the hybridized mRNA as a template. In some embodiments, the reverse transcription reaction produces single stranded cDNA molecules each having a molecular tag and barcode associated with the cDNA, followed by amplification of cDNA to produce a double stranded cDNA that includes the sequences of the barcoded molecules.

In some embodiments, the plurality of nucleic acid barcoded molecules comprise an oligo(dT) sequence. In that embodiment, the engineered family B polymerase reverse transcribes the mRNA molecule into a complementary DNA molecule using the mRNA hybridized to the oligo(dT) sequence of the nucleic acid barcoded molecules as a template, and the nucleic acid binding domain binds and stabilizes the mRNA-oligo(dT) hybrid during the reverse transcription. Following reverse transcription, the engineered transcriptase enzyme as described herein further amplifies the complementary DNA molecule comprising the barcode sequence, thereby generating an amplified DNA product comprising the barcode sequence, molecular tag sequence, or complements thereof.

In some embodiments of the nucleic acid extension method described herein, the method further comprises a second nucleic acid molecule comprising an oligo(dT) sequence. In that embodiment, the plurality of nucleic acid barcoded molecules further comprise an oligo(dT) sequence; and the nucleic acid binding domain of the engineered family B polymerase binds and stabilizes the mRNA-Oligo(dT) hybrid, while the engineered family B polymerase reverse transcribes the mRNA molecule using the second nucleic acid molecule comprising the oligo(dT) sequence, thereby generating a complementary DNA molecule. In this embodiment, the engineered family B polymerase further amplifies the complementary DNA molecule, thereby generating an amplified DNA product comprising a barcode sequence.

In some embodiments, the nucleic acid extension method further comprises a cell, a population of cells, or a tissue and the template nucleic acid molecule is from the cell, population of cells or the tissue.

### 3. Volume

In some embodiments, the engineered reverse transcriptase enzymes or derivatives thereof as described herein are used in a reaction volume less than about 1 nanoliter (nL). In some embodiments, the engineered reverse transcriptase enzymes or derivatives thereof as described herein are used in a reaction volume is less than about 500 picoliter (pL). In some embodiments, the reaction volume is contained within a partition. In some embodiments, the reaction volume is contained within a droplet. In some embodiments, the reaction volume is contained within a droplet in an emulsion. In some embodiments, the reaction volume is contained within a droplet emulsion having a reaction volume of less than about 1 nL. In some embodiments, the reaction volume is contained within a droplet emulsion having a reaction volume of less than about 500 pL. In some embodiments, the reaction volume is contained within a well. In some embodiments, the reaction volume is contained within a well having a reaction volume less than about 1 nL. In some embodiments, the reaction volume is contained within a well. In some embodiments, the reaction volume is contained within a well having a reaction volume less than about 500 pL. In some embodiments, the reaction volume is contained within a well in an array of wells having an extracted nucleic acid molecule, and where the template nucleic acid molecule is the extracted nucleic acid molecule. In some embodiments, the reaction volume is contained within a well in an array of wells having a cell comprising a template nucleic acid molecule, and where the template nucleic acid molecule is released from the cell.

### 4. Gel Bead

In some embodiments of the nucleic acid extension method described herein, the plurality of nucleic acid barcoded molecules are attached to a support (e.g., a particle, a slide, a chip, a bead, etc.). In one embodiment, the support is selected from the group consisting of an array, a bead, a gel bead, a microparticle, and a polymer. In some embodiments, the nucleic acid barcoded molecules attached to a support comprise molecular tags (UMIs), primer sequences, capture sequences, cleavage sequences, or additional functional sequences. In some embodiments, the support is a gel bead. In that embodiment, the nucleic acid barcoded molecules are releasably attached to the gel bead. In some embodiments, the gel bead comprises a polyacrylamide polymer.

In some embodiments, a cross-section of the gel bead is less than about 100 μm. In some embodiments, a cross-section of a gel bead is less than about 60 μm. In some embodiments, a cross-section of a gel bead is less than about 50 μm. In some embodiments, a cross-section of a gel bead is less than about 40 μm. In some embodiments, a cross-section of a gel bead is less than about 100 μm, less than about 99 μm, less than about 98 μm, less than about 97 μm, less than about 96 μm, less than about 95 μm, less than about 94 μm, less than about 93 μm, less than about 92 μm, less than about 91 μm, less than about 90 μm, less than about 89 μm, less than about 88 μm, less than about 87 μm, less than about 86 μm, less than about 85 μm, less than about 84 μm, less than about 83 μm, less than about 82 μm, less than about 81 μm, less than about 80 μm, less than about 79 μm, less than about 78 μm, less than about 77 μm, less than about 76 μm, less than about 75 μm, less than about 74 μm, less than about 73 μm, less than about 72 μm, less than about 71 μm, less than about 70 μm, less than about 69 μm, less than about 68 μm, less than about 67 μm, less than about 66 μm, less than about 65 μm, less than about 64 μm, less than about 63 μm, less than about 62 μm, less than about 61 μm, or less than about 60 μm.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 sequence for use in Illumina sequencing workflows. In some cases, the primer can comprise an R2 sequence for use in Illumina sequencing workflows. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference. However, the present disclosure is not limited as to a composition of any nucleic acid molecule or derivative thereof, or any particular sequencing platform and these characterizations serve as examples only which may be useful in a reverse transcription workflow.

In operation, a cell can be co-partitioned along with a barcode bearing bead. The barcoded nucleic acid molecules affixed to a bead can be released from the bead in the partition. By way of example, in the context of analyzing sample RNA, the poly-dT (poly-deoxythymine, also referred to as oligo (dT)) segment of one of the released nucleic acid molecules can hybridize to (e.g., capture)_the poly-A tail of a mRNA molecule. Reverse transcription may result in a cDNA transcript of the mRNA which cDNA transcript also includes each of the sequence segments of the nucleic acid molecule. Because the nucleic acid molecule comprises additional functional sequences (e.g., capture domains, primer domains, UMIs, barcodes, etc.), it can hybridize to and prime reverse transcription of the mRNA using the hybridized mRNA as a template. Within any given partition, all of the cDNA transcripts of the individual mRNA molecules may include a common barcode sequence. However, the transcripts made from the different mRNA molecules within a given partition may vary with respect to unique molecular identifying sequences (e.g., UMIs). Beneficially, following any subsequent amplification of the contents of a given partition, the number of different UMIs can be indicative of the quantity of mRNA originating from a given partition, and thus from the cell. As noted above, the transcripts can be amplified and sequenced to identify the sequence of the original mRNA captured template, as well as the sequence of the associated barcode and UMI. While a poly-dT capture sequence is described, other targeted or random capture sequences may also be used in capture or hybridize to a template for initiating the reverse transcription reaction.

Additional methods and systems for characterizing nucleic acids from small populations of cells, and in some cases, for characterizing nucleic acids from individual cells, especially in the context of larger populations of cells using the engineered family B polymerase of the present disclosure are known to those of skill in the art. See e.g., U.S. Patent Publication Nos. 2015/0376609, 2019/0367997; 2019/0064173, and 2021/0115415; and International Application Nos. PCT/US2020/17785, and PCT/US2018/016019. The methods and systems provide advantages of being able to provide the attribution advantages of the non-amplified single molecule methods with the high throughput of the other next generation systems, with the additional advantages of being able to process and sequence extremely low amounts of input nucleic acids derivable from individual cells or small collections of cells.

### C. RTL and Gap Filing

The present disclosure provides methods for use in various sample processing and analysis applications. The methods provided herein may involve hybridizing a probe to a target region of a nucleic acid molecule of interest, barcoding the resultant complex, and performing an extension, denaturation, and amplification processes to provide nucleic acid molecules comprising a sequence the same or substantially the same as or complementary to that of the target region of the nucleic acid molecule of interest.

The method may comprise hybridizing a first probe and a second probe to first and second target regions of the nucleic acid molecule, linking the first and second probes to provide a probe-linked nucleic acid molecule, and barcoding the probe-linked nucleic acid molecule.

RTL methods and application for analysis of nucleic acids from fresh and in fixed single cells are described in e.g., U.S. Pat. No. 10,208,343, and publication Chromium Fixed RNA Profiling Reagent Kits, User Guide CG000477 (e.g., RevD updated Feb. 14, 2023) which contents are incorporated by reference in its entirety.

RTL methods and applications for spatial analysis of nucleic acids, in fixed and/or fresh tissues are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in their entireties. The contents of each of these publications are herein incorporated by reference in their entirety.

Further non-limiting aspects of spatial analysis methodologies and compositions are described herein.

In some embodiments, a biological sample, e.g., cells in suspension, and/or a tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample comprises single cells. In some embodiments, the biological sample is a cell pellet, e.g., a fixed cell pellet, e.g., an FFPE cell pellet. FFPE samples are used in some instances in the RTL methods disclosed herein.

A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

In some embodiments, the first probe and the second probe hybridize to a first target region and a second target region which are adjacent to each other. In other embodiments, the first probe and the second probe are designed to hybridize to a first target region and a second target region which are not adjacent to each other. In certain embodiments the first and the second target regions are separated by 1-1000, 1-750, 1-500, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides; or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides.

One or more processes of the methods provided herein may be performed within a partition such as a droplet or well. The methods of the present disclosure may obviate the need for reverse transcription to generate cDNA, other than the cDNA generated during gap fill reaction between a first and a second probe, during analysis of ribonucleic acid molecules and may be useful, for example, in controlled analysis and processing of analytes such as biological particles, nucleic acids, and proteins.

### 1. Single RTL

Synthetic Biology relies on the ability to build novel DNAs from component parts. Double strand (ds) DNA molecules have been assembled by creating staggered ends at the both ends of a first DNA duplex. This has been achieved using restriction endonucleases or by using exonuclease digestion or by a wild-type DNA polymerase (e.g., a T4 polymerase) followed by hybridization and optional ligation of a second DNA duplex to the first duplex. Alternatively, to generate a ligation product using exonucleases and ligases in a reaction mixture (e.g., RTL), a non-strand displacing polymerases or RT is preferred.

In the RTL assay described herein, an enzyme lacking strand displacement is important to ensure that a hybridized oligonucleotide and/or probes are not removed by the polymerase during the extension of a first oligonucleotide. Without strand displacement, only the most 3′-directed primer to the preselected region is successfully extended to the location corresponding to the first primer.

An example of a non-strand displacing enzyme includes Phusion® polymerase (Thermo Fisher, Waltham, MA) (which is generally described as non-strand displacing), 9°N, Vent® or Pfu DNA polymerases. Additional DNA polymerases without strand displacement activity include T7, Q5 or T4 DNA polymerase. Indeed, as shown in FIGS. 3-4, non-strand displacing polymerases usually terminate synthesis of a template DNA when it encounters a blocking oligonucleotide.

Since DNA polymerases with strand displacement activity can displace a DNA oligonucleotide from a template strand of DNA at least as good as dissolving secondary or tertiary structure, the hybridization of the oligonucleotide and gap filling can be enhanced by using a non-strand displacing enzyme. In addition, the non-strand displacing requirement is necessary for successful post gap-fill ligation. Ligation typically does not occur if a portion of the probe is displaced, though a flap-endonuclease for example FEN1 endonuclease, could help remove the flap if some displacement occurs.

One aspect of the present disclosure provides a method of analyzing a sample comprising a nucleic acid molecule, the method comprising, consisting of, or consisting essentially of: (a) providing: (i) a sample comprising the nucleic acid molecule; (ii) a first probe comprising a first probe sequence and a second probe sequence; and (iii) a second probe comprising a third probe sequence; (b) subjecting the sample to conditions sufficient to (i) hybridize the first probe sequence of the first probe to the first target region of the nucleic acid molecule, and (ii) hybridize the third probe sequence of the second probe to the second target region of the nucleic acid molecule, such that the first reactive moiety of the first probe sequence of the first probe is adjacent to the second reactive moiety of the third probe sequence of the second probe; (c) subjecting the first reactive moiety and the second reactive moiety to conditions sufficient to yield a probe-linked nucleic acid molecule comprising the first probe linked to the second probe; and (d) barcoding the probe-linked nucleic acid molecule to generate a barcoded probe-linked nucleic acid.

In some embodiments, the method further comprises (e) optionally processing the nucleic acid to generate sequencing library from the barcoded probe-linked nucleic acids; (f) determining sequences from the sequencing library, and (g) correlating determined sequences with specific samples and/or partitions.

In step (a)(i), the nucleic acid molecule can comprise a first target region and a second target region, where the first target region is adjacent to the second target region. In step (a)(ii), the first probe sequence of the first probe is complementary to the first target region of the nucleic acid molecule, and the first probe sequence comprises a first reactive moiety. In step (a)(iii), the third probe sequence of the second probe can be complementary to the second target region of the nucleic acid molecule, and the third probe sequence can comprise a second reactive moiety.

In step (b), the first reactive moiety of the first probe sequence of the first probe can be separated from the second reactive moiety of the second probe sequence of the second probe. For example, the first reactive moiety of the first probe sequence of the first probe can be separated from the second reactive moiety of the second probe sequence of the second probe. by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides.

Alternatively, the first reactive moiety of the first probe sequence of the first probe can be separated from the second reactive moiety of the second probe sequence of the second probe by 1-1000, 1-900, 1-800, 1-700, 1-600, 1-500, 1-400, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides.

The first probe can be separated from the second probe by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides.

Optionally the first probe and the second probe that are so separated are part of the same molecule. In some embodiments, the first probe and the second probe that are so separated are part of different molecules.

In some embodiments, when the first probe can be so separated from the second reactive moiety of the second probe sequence of the second probe, the method further can comprise a gap fill reaction in the presence of an engineered family B polymerase. In that embodiment, the engineered family B polymerase can be an engineered recombinant Family-B polymerases (e.g., engineered family B polymerases; engineered DNA polymerase enzymes; engineered polymerases) that has reverse transcriptase activity and substantially lacks strand displacement activity.

The engineered family B polymerase can comprise an amino acid sequence having at least 75% sequence identity to the amino acid sequence of Pyrococcus furiosus (pfu) polymerase (SEQ ID NO: 1). The engineered family B polymerase can comprise an amino acid sequence having at least 75% sequence identity to the amino acid sequence of Thermococcus gorgonarius polymerase (Tgo polymerase) (SEQ ID NO: 10). The engineered family B polymerase can comprise an amino acid sequence having at least 75% sequence identity to the amino acid sequence of Thermococcus kodakarensis (KOD1) polymerase (SEQ ID NO: 6 or 8). The engineered family B polymerase can comprise an amino acid sequence having at least 75% sequence identity to the amino acid sequence of Thermococcus litoralis (VENT®) polymerase (SEQ ID NO: 20). The engineered family B polymerase can comprise an amino acid sequence having at least 75% sequence identity to the amino acid sequence of Pyrococcus sp. (Deep Vent) polymerase (SEQ ID NO: 21). The engineered family B polymerase can comprise an amino acid sequence having at least 75% sequence identity to the amino acid sequence of Thermococcus sp. (9°N) polymerase (SEQ ID NO: 22). The engineered family B polymerase can comprise an amino acid sequence having at least 75% sequence identity to the amino acid sequence of Thermococcus argininiproducens (Targ) polymerase (SEQ ID NO: 31). Optionally, the engineered family B polymerase has no detectable strand displacement activity as described herein.

The engineered family B polymerase can comprise an amino acid sequence that has at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31. The engineered family B polymerase can comprise an amino acid sequence that has at least 95% identity to the amino acid sequence of SEQ ID NO: 1, 6, 8, 10, 20-22, or 31. The engineered family B polymerase can comprise an amino acid sequence that has at least 97% identity to the amino acid sequence of SEQ ID NO: 1, 6, 8, 10, 20-22, or 31. The engineered family B polymerase can comprise an amino acid sequence that has at least about 10, at least about 15, at least about 16, at least about 18, at least about 20, at least about 25, or at least about 30 substitutions in the amino acid sequence of SEQ ID NO: 1. The engineered family B polymerase can comprise an amino acid sequence that has at least 97% identity to the amino acid sequence of SEQ ID NO: 1, 6, 8, 10, 20-22, or 31 and at least about 16 substitutions in the amino acid sequence of SEQ ID NO: 1, 6, 8, 10, 20-22, or 31. The engineered family B polymerase can comprise an amino acid sequence that has 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 6, 8, 10, 20-22, or 31.

The engineered family B polymerase can comprise the amino acid sequence set forth in SEQ ID NO: 2-5, 11, 12, and 25-30. The engineered family B polymerase can comprise an amino acid sequence having at least 90% sequence identity to the amino acid sequence set forth in SEQ ID NO: 2-5, 11, 12, and 25-30.

In some embodiments of the method of analyzing a sample comprising a nucleic acid molecule described herein, the gap fill reaction can be conducted in bulk and/or a partition. In some embodiments, the partition is a droplet, a well, a cell and/or a nucleus. In some embodiments, the sample is fixed.

In some embodiments of step (d), the probe-linked nucleic acid molecule is in a partition, and under suitable conditions and in some embodiments comprising a partition specific barcode to generate a barcoded probe-linked nucleic acid molecule.

Optionally, the partition can comprise a partition specific barcode to generate a barcoded probe-linked nucleic acid molecule, and the partition can comprise a single cell, a single nucleus, nucleic acids from a single cell, single cell nuclei, or a combination thereof.

In some embodiments, where the first and second probes are not immediately adjacent to each other, step (c) can comprise a gap fill reaction in the presence of one of the engineered family B polymerases (e.g., engineered family B polymerases; engineered DNA polymerase enzymes; engineered polymerases) of the disclosure. In that embodiment, the first probe and the second probe can be part of the same molecule. In some embodiments, the first probe and the second probe that can be part of different molecules.

In the gap fill reactions, an enzyme without strand displacing activity is desirable so as to fill in the gap between a first and a second probe which are not immediately adjacent to each other, without displacing the second/right hand side probe which may contain additional sequences which are not part of the nucleic acid target. Such additional sequences may include without limitation functional sequences such as constant sequence, probe barcode, and/or various capture sequences, or spatial capture sequences.

Particular embodiments of the methods, where the first and second probe are not immediately adjacent to each other, include without limitation applications for detection of variations in sequences between the first and second probes/the first and second targets. In that embodiment, the first and second probes can be operably linked to each other. For example, the first and second probes can be part of the same molecule. Such application include without limitation SNP detection, detection of insertions and/or deletions, and so forth.

In some embodiments, where a partition comprises multiple cells, the cells comprise any suitable barcode and/or index that permits computationally identifying nucleic acids that originated from a single cell and/or nucleus.

In some embodiments of the method of analyzing a sample comprising a nucleic acid molecule described herein, the first probe, the second probe, or the first and second probes comprise additional sequences selected from probe specific barcode sequences, UMI, or any further sequences for nucleic acid processing and sequencing library generation.

In some embodiments, the steps (a), (b) and (c) are conducted in bulk. In some embodiments, steps (a) and (b) are conducted in bulk, and (c) is conducted in a partition.

Another aspect of the present disclosure provides a method of analyzing a sample comprising a nucleic acid molecule, comprising, consisting essentially of, or consisting of: (a) providing: (i) a sample comprising the nucleic acid molecule, where the nucleic acid molecule comprises a first target region and a second target region, optionally where in some embodiments the first target region is adjacent to the second target region; (ii) a first probe comprising a first probe sequence, and optionally another probe sequence, where the first probe sequence of the first probe is complementary to the first target region of the nucleic acid molecule, and where the first probe sequence comprises a first reactive moiety; and (iii) a second probe comprising a second probe sequence, where the second probe sequence of the second probe is complementary to the second target region of the nucleic acid molecule, and where the second probe sequence comprises a second reactive moiety; (b) subjecting the sample to conditions sufficient to (i) hybridize the first probe sequence of the first probe to the first target region of the nucleic acid molecule, and (ii) hybridize the second probe sequence of the second probe to the second target region of the nucleic acid molecule, such that the first reactive moiety of the first probe sequence of the first probe is separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe; (c) subjecting the first reactive moiety and the second reactive moiety to conditions sufficient to yield a probe-linked nucleic acid molecule comprising the first probe linked to the second probe; in non-limiting embodiments, suitable conditions include contacting the first reactive moiety and the second reactive moiety with a ligase; and (d) barcoding the probe-linked nucleic acid molecule to generate a barcoded probe-linked nucleic acid.

In some embodiments, methods described herein include ligating an extended first probe to a second probe. In some embodiments, the ligating utilizes a ligase. In some embodiments, the ligase is a DNA ligase. The ligase can comprise a family B ligase. The ligase can be selected from the group consisting of T4 DNA ligase, T4 RNA ligase, Chlorella virus DNA ligase, Paramecium bursaria Chlorella virus 1 DNA ligase I (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase. the engineered family B polymerase.

In some embodiments, the method further comprise additional steps of nucleic acid processing to generate sequencing library from the barcoded probe-linked nucleic acids, determining sequences from the sequencing library, and correlating determined sequences with specific samples and/or partitions. In some embodiments of (d), the probe-linked nucleic acid molecule is in a partition, and under suitable conditions. In some embodiments the partition comprises a partition specific barcode to generate a barcoded probe-linked nucleic acid molecule.

In some embodiments, the partition comprises a single cell, single nucleus, nucleic acids from a single cell and/or single cell nucleus, or a combination of single cells, single nuclei, and/or nucleic acids from these. In some embodiments of the methods, where a partition comprises multiple cells, e.g., multiplexing, the cells comprise any suitable barcode and/or index that permits computationally identifying nucleic acid(s) that originated from a single cell and/or nucleus. In some embodiments of the methods, one or both of the probes comprise additional sequences, including without limitation probe specific barcode sequence(s), UMI, and any further sequences for nucleic acid processing, and sequencing library generation.

In some embodiments of the method of analyzing a sample described herein, steps (a), (b) and (c) are conducted in bulk. Alternatively, steps (a) and (b) are conducted in bulk, and (c) is conducted in a partition. In some embodiments, when the first probe is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides, the method further comprises a gap fill reaction in the presence of one of the engineered family B polymerases (e.g., engineered family B polymerases; engineered DNA polymerase enzymes; engineered polymerases) of the disclosure. In some embodiments, the engineered family B polymerase is an engineered Tgo enzyme or a variant thereof (FIGS. 3A-B).

In some embodiments of the method of analyzing a sample, the enzyme is any of the engineered family B polymerases described herein. In some embodiments, the engineered family B polymerase comprises the amino acid sequence set forth in SEQ ID NO: 2-5, 11, 12, and 25-30 or an amino acid sequence having at least 90% sequence identity to the amino acid sequence set forth in SEQ ID NO: 2-5, 11, 12, and 25-30. In some embodiments, the engineered family B polymerase comprises an amino acid that is at least 90% identical to SEQ ID NO: 10, 11 or 12. In some embodiments, the engineered family B polymerase comprises the amino acid of SEQ ID NO: 11. In some embodiments, the engineered family B polymerase comprises the amino acid of SEQ ID NO: 11.

In some embodiments, the gap fill reaction is conducted in bulk and/or a partition. In certain embodiments, the partition is a droplet, a well, a cell and/or a nucleus. In certain embodiments, the cell and/or nucleus is fixed.

Another aspect of the present disclosure provides a method for analyzing a target nucleic acid in a biological sample, the method comprising, consisting of, or consisting essentially of: (a) contacting the biological sample with a first probe comprising a first probe sequence, and optionally another probe sequence, where the first probe sequence of the first probe is complementary to a first target region of the nucleic acid molecule, and where the first probe sequence comprises a first reactive moiety; and (iii) a second probe comprising a second probe sequence, where the second probe sequence of the second probe is complementary to a second target region of the nucleic acid molecule, and where the second probe sequence comprises a second reactive moiety, (b) hybridizing the plurality of first probe oligonucleotide to the first target region and the plurality of second probe oligonucleotide to the second target region, such that the first reactive moiety of the first probe sequence of the first probe is separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe; (c) subjecting the first reactive moiety and the second reactive moiety to conditions sufficient to yield a probe-linked nucleic acid molecule comprising the first probe linked to the second probe; in non-limiting embodiments, suitable conditions include contacting the first reactive moiety and the second reactive moiety with a ligase; (d) (optionally) releasing the probe-linked nucleic acid molecule from the target nucleic acid; (e) contacting the probe-linked nucleic acid molecule with a substrate comprising a plurality of capture probes, to hybridize the probe-linked nucleic acid molecule to a capture domain of the capture probe which is affixed to the substrate; (f) further processing the hybridized probe-linked nucleic acid molecule(s) to generate a sequencing library, (g) determining sequences of the probe-linked nucleic acid molecules in the sequencing library or a complement thereof, and (h) using the determined sequence(s) identifying the location of the in the biological sample. In some embodiments, of this method, the plurality of first probe oligonucleotides and the plurality of second probe oligonucleotides target a plurality of nucleic acids in the biological sample.

In step (d), the ligase can be any ligase. The ligase can comprise a family B ligase. The ligase can be selected from the group consisting of T4 DNA ligase, T4 RNA ligase, Chlorella virus DNA ligase, Paramecium bursaria Chlorella virus 1 DNA ligase I (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase. Alternatively, the ligase can comprise a single stranded DNA ligase, or an Archaeal RNA ligase. The ligase can also be from the same species as the engineered family B polymerase described herein.

In some embodiments, each first probe and each second probe of the plurality comprise sequences are substantially complementary to a target nucleic acid in the biological sample, and each second probe of the plurality comprises a capture probe domain sequence. In some embodiments, the sample is fixed to a solid support, e.g., a slide, and method determines spatial position of the target nucleic acids in the sample.

In some embodiments of the method for analyzing a target nucleic acid in a biological sample the first probe is separated from the second probe by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95. In some embodiments of the method for analyzing a target nucleic acid in a biological sample when the first probe is separated from the second probe 100 nucleotides, or by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides. In that embodiment, the first probe and the second probe can be part of the same molecule. In some embodiments, the first probe and the second probe that can be part of different molecules.

In some embodiments of the method for analyzing a target nucleic acid in a biological sample when the first probe is separated from the second probe as described herein, the method further comprises a gap fill reaction in the presence of one of the engineered family B polymerases (e.g., engineered family B polymerases; engineered DNA polymerase enzymes; engineered polymerases) of the disclosure.

In some embodiments, the enzyme is any of the enzymes in the preceding claims. In some embodiments, the engineered family B polymerase comprises the amino acid sequence set forth in SEQ ID NO: 2-5, 11, 12, and 25-30 or an amino acid sequence having at least 90% sequence identity to the amino acid sequence set forth in SEQ ID NO: 2-5, 11, 12, and 25-30. In some embodiments of the methods, one or both of the probes comprise additional sequences, including without limitation probe specific barcode sequence(s), UMI, or any further sequences for nucleic acid processing, and sequencing library generation.

### 2. Spatial RTL

One aspect of the present disclosure provides a method for determining a location of a target nucleic acid in a biological sample, the method comprising, consisting of, or consisting essentially of (a) contacting the biological sample with a plurality of first probe oligonucleotides and a plurality of second probe oligonucleotides, (b) hybridizing the plurality of first probe oligonucleotide and the plurality of second probe oligonucleotide to the target nucleic; (c) extending each first probe oligonucleotide of the plurality using an engineered family B polymerase of the disclosure, e.g. a non-strand displacing reverse transcriptase, to generate an extended first probe oligonucleotide, thereby filling in a gap between the first probe oligonucleotide and the second probe oligonucleotide; (d) optionally cleaving the sequence of non-complementary nucleotides; (e) ligating the extended first probe oligonucleotide and the second probe oligonucleotide, thereby creating a ligated probe that is substantially complementary to the target nucleic acid; (f) releasing the ligated probe from the target nucleic acid; (g) contacting the biological sample with a substrate comprising a plurality of capture probes; (h) hybridizing the ligation product to the capture domain of the capture probe affixed to the substrate; and (i) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the location of the analyte in the biological sample. In some embodiments, of this method, the plurality of first probe oligonucleotides and the plurality of second probe oligonucleotides target a plurality of nucleic acids in the biological sample. In some embodiments, each first probe and each second probe of the plurality comprise sequences are substantially complementary to a target nucleic acid in the biological sample, and each second probe of the plurality comprises a capture probe domain sequence. In some embodiments, each first probe oligonucleotide and each second probe oligonucleotide of the plurality hybridize to sequences that are not adjacent to each other on the plurality of target nucleic acids.

In step (c), each first probe oligonucleotide of the plurality using an engineered family B polymerase that has no detectable strand displacement activity. the engineered family B polymerase can be an engineered recombinant Family-B polymerases (e.g., engineered family B polymerases; engineered DNA polymerase enzymes; engineered polymerases) that has reverse transcriptase activity and substantially lacks strand displacement activity.

The engineered family B polymerase can comprise an amino acid sequence having at least 75% sequence identity to the amino acid sequence of Pyrococcus furiosus (pfu) polymerase (SEQ ID NO: 1). The engineered family B polymerase can comprise an amino acid sequence having at least 75% sequence identity to the amino acid sequence of Thermococcus gorgonarius polymerase (Tgo polymerase) (SEQ ID NO: 10). The engineered family B polymerase can comprise an amino acid sequence having at least 75% sequence identity to the amino acid sequence of Thermococcus kodakarensis (KOD1) polymerase (SEQ ID NO: 6 or 8). The engineered family B polymerase can comprise an amino acid sequence having at least 75% sequence identity to the amino acid sequence of Thermococcus litoralis (VENT®) polymerase (SEQ ID NO: 20). The engineered family B polymerase can comprise an amino acid sequence having at least 75% sequence identity to the amino acid sequence of Pyrococcus sp. (Deep Vent) polymerase (SEQ ID NO: 21). The engineered family B polymerase can comprise an amino acid sequence having at least 75% sequence identity to the amino acid sequence of Thermococcus sp. (9°N) polymerase (SEQ ID NO: 22). The engineered family B polymerase can comprise an amino acid sequence having at least 75% sequence identity to the amino acid sequence of Thermococcus argininiproducens (Targ) polymerase (SEQ ID NO: 31). Optionally, the engineered family B polymerase has no detectable strand displacement activity as described herein.

In some embodiments, each first probe oligonucleotide of the plurality is extended with an engineered family B polymerase, optionally the engineered family B polymerase comprises an amino acid sequence having at least 75% sequence identity to the amino acid sequence of Thermococcus gorgonarius polymerase (Tgo polymerase) or SEQ ID NO: 10.

In some embodiments of the method for determining a location of a target nucleic acid in a biological sample described herein, each first probe oligonucleotide of the plurality and each second probe oligonucleotide of the plurality are operably linked; or each first probe oligonucleotide of the plurality and each second probe oligonucleotide of the plurality are part of the same molecule.

In some embodiments of the method for determining a location of a target nucleic acid in a biological sample described herein, the engineered family B polymerase comprises an amino acid sequence having: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31; (b) at least 95% identity to the amino acid sequence of SEQ ID NO: 1, 6, 8, 10, 20-22, or 31; (c)
at least 97% identity to the amino acid sequence of SEQ ID NO: 1, 6, 8, 10, 20-22, or 31; (d) at least about 10, at least about 15, at least about 16, at least about 18, at least about 20, at least about 25, or at least about 30 substitutions in the amino acid sequence of SEQ ID NO: 1; (e) at least 97% identity to the amino acid sequence of SEQ ID NO: 1, 6, 8, 10, 20-22, or 31 and at least about 16 substitutions in the amino acid sequence of SEQ ID NO: 1, 6, 8, 10, 20-22, or 31; or (f) 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 6, 8, 10, 20-22, or 31.

In some embodiments, the engineered family B polymerase comprises an amino acid sequence selected from SEQ ID NO: 2-5, 11, 12, and 25-30; optionally the engineered family B polymerase is an engineered non-strand displacing reverse RT.

Generating the ligation product can comprise ligating the extended first probe to the second probe of the plurality using an enzymatic ligation or a chemical ligation, optionally the enzymatic ligation utilizes a ligase. The ligase can be any ligase. The ligase can comprise a family B ligase. The ligase can be selected from the group consisting of T4 DNA ligase, T4 RNA ligase, Chlorella virus DNA ligase, Paramecium bursaria Chlorella virus 1 DNA ligase I (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase. Alternatively, the ligase can comprise a single stranded DNA ligase, or an Archaeal RNA ligase. The ligase can also be from the same species as the engineered family B polymerase described herein.

In some embodiments of the method for determining a location of a target nucleic acid in a biological sample described herein, each first probe oligonucleotide of the plurality and each second probe oligonucleotide of the plurality hybridized to nucleic acid sequences can be about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other.

Each first probe oligonucleotide of the plurality and each second probe oligonucleotide of the plurality hybridized to nucleic acid sequences can be 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900, or 1000 nucleotides away from each other.

In that embodiment, each first probe oligonucleotide of the plurality and each second probe oligonucleotide of the plurality hybridized to nucleic acid sequences can be at least about 1-100, at least about 1-90, at least about 1-80, at least about 1-70, at least about 1-60, at least about 1-50, at least about 1-40, at least about 1-30, at least about 1-20, at least about 1-10, at least about 1-9, at least about 1-8, at least about 1-7, at least about 1-6, at least about 1-5, at least about 1-4, at least about 1-3, at least about 1-2 nucleotides apart; or (b) 1-100, 1-90, 1-80, 1-70, 1-60, 1-50, 1-40, 1-30, 1-20, 1-10, 1-9, 1-8, 1-7, 1-6, 1-5, 1-4, 1-3, or 1-2 nucleotides apart.

In some embodiments of the method for determining a location of a target nucleic acid in a biological sample described herein, the method further can comprise, consist of or consist essentially of extending a 3′ end of the capture probe using the ligation product. In that embodiment, extending the 3′ end of the capture probe comprises reverse transcribing the target nucleic acid using the engineered family B polymerase.

In some embodiments of the method for determining a location of a target nucleic acid in a biological sample described herein, the determining step (i) comprises amplifying all or part of the ligation product using the engineered family B polymerase. In that embodiment, the amplified product comprises (i) all or part of sequence of the ligation product, or a complement thereof, and (ii) the sequence of the spatial barcode, or a complement thereof.

In some embodiments, each capture probe of the plurality of capture probes of the substrate comprises: (i) a spatial barcode and (ii) a capture domain that comprises a sequence that is complementary to all or a portion of the capture probe domain of the second probe oligonucleotide. In some embodiments, generating the ligation product comprises ligating the extended first probe to the second probe using enzymatic ligation or chemical ligation. In some embodiments, the enzymatic ligation utilizes a ligase. In some embodiments of the method for determining a location of a target nucleic acid in a biological sample described herein, the method may further comprise extending a 3′ end of the capture probe using the ligation product. In some embodiments, extending the 3′ end of the capture probe comprises reverse transcribing the target nucleic acid using an engineered family B polymerase described herein. In some embodiments of the method for determining a location of a target nucleic acid in a biological sample described herein, the determining step can comprise amplifying all or part of the ligation product using an engineered family B polymerase described herein. In some embodiments, the amplified product can comprise (i) all or part of sequence of the ligation product, or a complement thereof, and (ii) the sequence of the spatial barcode, or a complement thereof.

### 3. RNA-Templated Ligation (RTL)

Templated ligation or RNA-templated ligation (RTL) is a process that includes multiple oligonucleotides (also called “oligonucleotide probes” or simply “probes,” and a pair of probes can be called interchangeably “first probes” and “second probes,” or “first probe oligonucleotides” and “second probe oligonucleotides,”) that hybridize to adjacent complementary analyte (e.g., mRNA) sequences. Upon hybridization, the two oligonucleotides are ligated to one another, creating a ligation product in the event that both oligonucleotides hybridize to their respective complementary sequences. In some instances, at least one of the oligonucleotides includes a sequence (e.g., a poly-adenylation sequence) that can be hybridized to a probe on an array described herein (e.g., the probe comprises a poly-thymine sequence in some instances). In some instances, prior to hybridization of the poly-thymine to the poly(A) sequence, an endonuclease digests the analyte that is hybridized to the ligation product. This step frees the newly formed ligation product to hybridize to a capture probe on a spatial array. In this way, templated ligation provides a method to perform targeted RNA capture on a spatial array. Improved methods for identifying a location of an analyte in a biological sample through a method that utilizes templated ligation of multiple (e.g., two) oligonucleotides are known in the art. See e.g., U.S. Pat. Nos. 11,608,520 and 11,332,790, which are incorporated herein by reference in their entirety.

Additional features of capture probes are described in WO 2020/176788 and/or U.S. Patent Application Publication No. 2020/0277663, each of which is incorporated by reference in its entirety. Generation of capture probes can be achieved by any appropriate method, including those described in WO 2020/176788 and/or U.S. Patent Application Publication No. 2020/0277663, each of which is incorporated by reference in its entirety.

### 4. Gap Filing

In some embodiments of the method described herein, the method utilizes templated ligation of multiple oligonucleotides (e.g., two) that hybridize to substantially complementary sequences that are not immediately adjacent to one another. For example, the complementary sequences to which the first probe oligonucleotide and the second probe oligonucleotide bind are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other.

Thus, in some embodiments of the methods disclosed herein, each first probe oligonucleotide and each second probe oligonucleotide hybridize to sequences that are about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other. In some embodiments, each first probe oligonucleotide and each second probe oligonucleotide hybridize to sequences that 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900, or 1000 nucleotides away from each other.

In some embodiments, the complementary sequences to which the first probe oligonucleotide and the second probe oligonucleotide bind comprises a gap between the hybridized probes of at least 1-100, 1-90, 1-80, 1-70, 1-60, 1-50, 1-40, 1-30, 1-20, 1-10, 1-9, 1-8, 1-7, 1-6, 1-5, 1-4, 1-3, 1-2 or 1 nucleotides. In some embodiments, each first probe oligonucleotide and each second probe oligonucleotide hybridize to sequences that are at least about 1-100, at least about 1-90, at least about 1-80, at least about 1-70, at least about 1-60, at least about 1-50, at least about 1-40, at least about 1-30, at least about 1-20, at least about 1-10, at least about 1-9, at least about 1-8, at least about 1-7, at least about 1-6, at least about 1-5, at least about 1-4, 1-3, at least about 1-2 nucleotides apart. Alternatively, each first probe oligonucleotide and each second probe oligonucleotide hybridize to sequences that are 1-100, 1-90, 1-80, 1-70, 1-60, 1-50, 1-40, 1-30, 1-20, 1-10, 1-9, 1-8, 1-7, 1-6, 1-5, 1-4, 1-3, or 1-2 nucleotides apart. In certain embodiments the first and the second target regions are separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides.

In some embodiments, gaps between the probe oligonucleotides may first be filled prior to ligation, using, for example, a DNA polymerase, an RNA polymerase, or a reverse transcriptase and/or any combinations, derivatives, and/or variants (e.g., any engineered family B polymerases of the present disclosure) thereof.

Since DNA polymerases with strand displacement activity can displace a DNA oligonucleotide from a template strand of DNA at least as good as dissolving secondary or tertiary structure, the hybridization of the oligonucleotide and gap filling can be enhanced by using a non-strand displacing enzyme. Indeed, as shown in FIGS. 3-4, non-strand displacing polymerases usually terminate synthesis of a template DNA when its encounters a blocking oligonucleotide. In the RTL assays described herein, the non-displacement characteristic is important to ensure that hybridized oligonucleotides or probes are not removed by the nucleic acid processing enzyme, e.g., polymerase and/or RT, during the extension of a first oligonucleotide.

Accordingly, in some embodiments of the methods described herein, the gap are filled using an engineered family B polymerase described herein. To fill the gap, each first probe oligonucleotide of the plurality of oligonucleotide that hybridize to the complementary sequences is extended with an engineered family B polymerase described herein. In some embodiments, the engineered family B polymerase can comprise an amino acid sequence having at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 10, 20-22, or 31. In some embodiments, the engineered family B polymerase can comprise at least 95% identity to the amino acid sequence of SEQ ID NO: 1, 10, 20-22, or 31. In some embodiments, the engineered family B polymerase can comprise at least 97% identity to the amino acid sequence of SEQ ID NO: 1, 10, 20-22, or 31. In some embodiments, the engineered family B polymerase can comprise at least about 10, at least about 15, at least about 16, at least about 18, at least about 20, at least about 25, or at least about 30 substitutions in the amino acid sequence of SEQ ID NO: 1. In some embodiments, the engineered family B polymerase can comprise at least 97% identity to the amino acid sequence of SEQ ID NO: 1, 10, 20-22, or 31 and at least about 16 substitutions in the amino acid sequence of SEQ ID NO: 1, 10, 20-22, or 31. In some embodiments, the engineered family B polymerase can comprise 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 10, 20-22, or 31.

In some embodiments of the method for determining a location of a target nucleic acid in a biological sample described herein, the gap between two oligonucleotides that hybridize to substantially complementary sequences that are not immediately adjacent to one another, may be filled with an engineered family B polymerase comprising an amino acid sequence selected from SEQ ID NO: 2-5, 11,12, and 25-30.

A biological sample including an analyte (e.g., a nucleic acid) can be contacted with a first probe and a second probe. The first probe and the second probe can hybridize to the analyte at a first target sequence and a second target sequence, respectively. After hybridization, unbound first and second probes are washed away. The first probe and the second probe can include free ends. In some embodiments, the first and second target sequences are immediately adjacent to each other, such that the hybridizes probes are immediately adjacent to each other (i.e., there is no nucleotide gap between the hybridized probes). In some embodiments, the first and second target sequences are not directly adjacent in the analyte, such that the hybridizes probes are not immediately adjacent to each other (i.e., there is a gap between the hybridized probes). In some embodiments, gap filling reaction is performed so that the gap between the two probes is filled. In some embodiments, the first probe can be extended to the second probe, and then a ligation product is created that includes the first probe sequence and the second probe sequence.

In some embodiments, the gap between the first and second probes is 1-1000, 1-750, 1-500, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides; or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides.

In some embodiments, a third oligonucleotide may be added that hybridizes to the first and the second probes. Alternatively, instead of extending the first probe, the third oligonucleotide is used to “bind” the first probe and the second probe together. In such cases, the first probe and the second probe bound together by the third oligonucleotide can be referred to as a ligation product. The ligation product then is contacted with a substrate, and the ligation product is bound to a capture probe of the substrate on the array at distinct spatial positions. In some embodiments, the biological sample is contacted with the substrate prior to being contacted with the first probe and the second probe.

### D. Biological Sample

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some embodiments, a biological sample (e.g., tissue section) can be fixed with methanol, stained with hematoxylin and eosin, and imaged. In some embodiments, fixing, staining, and imaging occurs before one or more oligonucleotide probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the methods of targeted RNA capture as disclosed herein include hybridization of multiple probe oligonucleotides. In some embodiments, the methods include 2, 3, 4, or more probe oligonucleotides that hybridize to one or more analytes of interest. In some embodiments, the methods include two probe oligonucleotides. In some embodiments, the probe oligonucleotide includes sequences complementary that are complementary or substantially complementary to an analyte. For example, in some embodiments, the probe oligonucleotide includes a sequence that is complementary or substantially complementary to an analyte (e.g., an mRNA of interest (e.g., to a portion of the sequence of an mRNA of interest)). Methods provided herein may be applied to a single nucleic acid molecule or a plurality of nucleic acid molecules. A method of analyzing a sample comprising a nucleic acid molecule may comprise providing a plurality of nucleic acid molecules (e.g., RNA molecules), where each nucleic acid molecule comprises a first target region (e.g., a sequence that is 3′ of a target sequence or a sequence that is 5′ of a target sequence) and a second target region (e.g., a sequence that is 5′ of a target sequence or a sequence that is 3′ of a target sequence), a plurality of first probe oligonucleotides, and a plurality of second probe oligonucleotides.

In some embodiments, the templated ligation methods that allow for targeted RNA capture as provided herein include a first probe oligonucleotide and a second probe oligonucleotide. The first and second probe oligonucleotides each include sequences that are substantially complementary to the sequence of an analyte of interest. By substantially complementary, it is meant that the first and/or second probe oligonucleotide is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in an analyte. In some instances, the first probe oligonucleotide and the second probe oligonucleotide hybridize to adjacent sequences on an analyte.

In some embodiments, the first and/or second probe as disclosed herein includes one of at least two ribonucleic acid bases at the 3′ end; a functional sequence; a phosphorylated nucleotide at the 5′ end; and/or a capture probe binding domain. In some embodiments, the functional sequence is a primer sequence. The capture probe binding domain is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or both. In some embodiments, the capture probe binding domain includes a random sequence (e.g., a random hexamer or octamer). In some embodiments, the capture probe binding domain is complementary to a capture domain in a capture probe that detects a particular target(s) of interest.

In some embodiments, a capture probe binding domain blocking moiety that interacts with the capture probe binding domain is provided. In some instances, the capture probe binding domain blocking moiety includes a nucleic acid sequence. In some instances, the capture probe binding domain blocking moiety is a DNA oligonucleotide. In some instances, the capture probe binding domain blocking moiety is an RNA oligonucleotide. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a ligated probe) to a capture probe. In some embodiments, a capture probe binding domain blocking moiety comprises a poly-uridine sequence, a poly-thymidine sequence, or both.

In some embodiments, the first probe oligonucleotide hybridizes to an analyte. In some embodiments, the second probe oligonucleotide hybridizes to an analyte. In some embodiments, both the first probe oligonucleotide and the second probe oligonucleotide hybridize to an analyte. Hybridization can occur at a target having a sequence that is 100% complementary to the probe oligonucleotide(s). In some embodiments, hybridization can occur at a target having a sequence that is at least (e.g., at least about) 80%, at least (e.g., at least about) 85%, at least (e.g., at least about) 90%, at least (e.g., at least about) 95%, at least (e.g., at least about) 96%, at least (e.g. at least about) 97%, at least (e.g., at least about) 98%, or at least (e.g., at least about) 99% complementary to the probe oligonucleotide(s).

After hybridization of the first and second probe oligonucleotides, in some embodiments, the first probe oligonucleotide is extended. After hybridization, in some embodiments, the second probe oligonucleotide is extended. Extending probes can be accomplished using any method disclosed herein. In some instances, a polymerase (e.g., a DNA polymerase) extends the first and/or second oligonucleotide.

In some embodiments, methods disclosed herein include a wash step. In some instances, the wash step occurs after hybridizing the first and the second probe oligonucleotides. The wash step removes any unbound oligonucleotides and can be performed using any technique or solution disclosed herein or known in the art. In some embodiments, multiple wash steps are performed to remove unbound oligonucleotides.

In some embodiments, after hybridization of probe oligonucleotides (e.g., first and the second probe oligonucleotides) to the analyte, the probe oligonucleotides (e.g., the first probe oligonucleotide and the second probe oligonucleotide) are ligated together, creating a single ligated probe that is complementary to the analyte. Ligation can be performed enzymatically or chemically, as described herein.

### E. Additional Embodiments

Another aspect of the present disclosure provides a method of using an engineered family B polymerase described herein, the method comprising, consisting essentially of or consisting of contacting the engineered family B polymerase with a plurality of nucleic acid templates under suitable conditions to produce a polymerized nucleic acid product, where the plurality of nucleic acid templates can be a plurality of RNAs, DNAs, or nucleic acids comprising an unnatural nucleotide.

In certain embodiments, methods of using the engineered family B polymerases comprise providing a nucleic acid template, a first probe and a second probe which are hybridized and/or designed to hybridize to a first and a second target nucleic acid/target region in the nucleic acid template, e.g., mRNA. In certain embodiments of the method, the polymerized product produced by the engineered family B polymerases is generated between a first probe and a second probe hybridized to a first target sequence/target region and a second target sequence/target region, where the target sequences and/or the probes hybridized to these are not immediately adjacent to each other, e.g., there is more than zero nucleotides between the target sequences and/or the probes. In certain embodiments of the method, the polymerized product is generated between a first probe and a second probe hybridized to a first target sequence/target region and a second target sequence/target region, where the target sequences and/or the probes hybridized to these are separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, by 1-1000, 1-750, 1-500, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides, by 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 200, 300, 400, 500, 600, 700, 800, 900, or 1000 nucleotides; or by about: 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides.

In some embodiments, the plurality of nucleic acid templates can be located in a biological sample. In some embodiments, the biological sample: (is a Formalin-Fixed Paraffin-Embedded (FFPE) sample, a formalin-fixed sample, a paraffin-embedded sample, a frozen sample, or a fresh sample; (b) a single cell and/or a nucleus, for example in a suspension and/or from homogenized, which could be fresh, frozen, permeabilized, and/or fixed by any suitable fixative, including PFA, and/or (c) a tissue.

Another aspect of the present disclosure provides a nucleic acid extension method comprising: (a) contacting a target nucleic acid molecule with an engineered family B polymerase and a plurality of nucleic acid molecules, including without limitation nucleic acid barcoded molecules comprising a barcode sequence, and (b) incubating the target nucleic acid, the engineered family B polymerase and barcoded molecules under conditions in which the barcoded molecules are extended by the engineered family B polymerase; where: (i) the engineered family B polymerase comprises an amino acid sequence that has at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 10, 20-22, or 31; (ii) one of the plurality of nucleic acid barcoded molecules hybridizes to the target nucleic acid molecule; and (iii) the engineered family B polymerase extends the one of the plurality of nucleic acid barcoded molecules that is hybridized to the target nucleic acid molecule.

In some embodiments of the nucleic acid extension method: (a) the nucleic acid is a ribonucleic acid (RNA) molecule; and (b) the engineered family B polymerase reverse transcribes the RNA molecule into a complementary DNA, and then amplifies the complementary DNA into a nucleic acid product in the same reaction.

In some embodiments, the RNA molecule is a messenger RNA (mRNA) molecule. In some embodiments: (a) the plurality of nucleic acid barcoded molecules further comprise an oligo(dT) sequence; and (b) the engineered family B polymerase reverse transcribes the mRNA molecule into a complementary DNA (cDNA) molecule using the mRNA hybridized to the oligo(dT) sequence of the nucleic acid barcoded molecules as a template, thereby generating a complementary DNA molecule comprising the barcode sequence.

In some embodiments, the engineered family B polymerase further amplifies the complementary DNA molecule comprising the barcode sequence, thereby generating an amplified DNA product comprising the barcode sequence or complements thereof.

In some embodiments: (a) the method further comprises a second nucleic acid molecule comprising an oligo(dT) sequence; (b) the plurality of nucleic acid barcoded molecules further comprises an oligo(dT) sequence; and (c) the engineered reverse transcribes the mRNA molecule using the second nucleic acid molecule comprising the oligo(dT) sequence, thereby generating a complementary DNA molecule.

In some embodiments, the engineered family B polymerase further amplifies the complementary DNA molecule using the plurality of nucleic acid barcoded molecules, thereby generating an amplified DNA product comprising a barcode sequence. In some embodiments: (a) the plurality of nucleic acid barcoded molecules are attached to a support; and (b) the support is selected from the group consisting of an array, a bead, a gel bead, a microparticle, and a polymer.

In some embodiments, the engineered family B polymerase comprises the amino acid sequence set forth in SEQ ID NO: 2-5, 11, 12, and 25-30 or an amino acid sequence having at least 90% sequence identity to the amino acid sequence set forth in SEQ ID NO: 2-5, 11, 12, and 25-30.

Another aspect of the present disclosure provides a method for determining a location of a target nucleic acid in a biological sample, the method comprising (a) contacting the biological sample with a plurality of first probe oligonucleotides and a plurality of second probe oligonucleotides, where: (i) the plurality of first probe oligonucleotides and the plurality of second probe oligonucleotides target a plurality of nucleic acids in the biological sample, (ii) each first probe and each second probe of the plurality comprise sequences are substantially complementary to a target nucleic acid in the biological sample, and (iii) each second probe of the plurality comprises a capture probe domain sequence; (b) hybridizing the plurality of first probe oligonucleotide and the plurality of second probe oligonucleotide to the target nucleic, where each first probe oligonucleotide and each second probe oligonucleotide of the plurality hybridize to sequences that are not adjacent to each other on the plurality of target nucleic acids; (c) extending each first probe oligonucleotide of the plurality using an engineered non-strand displacing reverse transcriptase to generate an extended first probe oligonucleotide, thereby filling in a gap between the first probe oligonucleotide and the second probe oligonucleotide; (d) cleaving the sequence of non-complementary nucleotides; (e) ligating the extended first probe oligonucleotide and the second probe oligonucleotide, thereby creating a ligated probe that is substantially complementary to the target nucleic acid; (f) releasing the ligated probe from the target nucleic acid; (g) contacting the biological sample with a substrate comprising a plurality of capture probes, optionally each capture probe of the plurality of capture probes comprises: (i) a spatial barcode and (ii) a capture domain and optionally the capture domain comprises a sequence that is complementary to all or a portion of the capture probe domain of the second probe oligonucleotide; (h) hybridizing the ligation product to the capture domain of the capture probe affixed to the substrate; and (i) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the location of the analyte in the biological sample.

In some embodiments of the methods, generating a ligation product comprises ligating the extended first probe to the second probe using enzymatic ligation or chemical ligation, optionally the enzymatic ligation utilizes a ligase.

In some embodiments of the methods, each first probe oligonucleotide and each second probe oligonucleotide hybridize to sequences that are: (a) about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other; or (b) 0, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900, or 1000 nucleotides away from each other.

In some embodiments of the method for determining a location of a target nucleic acid in a biological sample described herein, each first probe oligonucleotide and each second probe oligonucleotide hybridize to sequences that are: (a) at least about 1-100, at least about 1-90, at least about 1-80, at least about 1-70, at least about 1-60, at least about 1-50, at least about 1-40, at least about 1-30, at least about 1-20, at least about 1-10, at least about 1-9, at least about 1-8, at least about 1-7, at least about 1-6, at least about 1-5, at least about 1-4, at least about 1-3, at least about 1-2 nucleotides apart; or (b) 1-100, 1-90, 1-80, 1-70, 1-60, 1-50, 1-40, 1-30, 1-20, 1-10, 1-9, 1-8, 1-7, 1-6, 1-5, 1-4, 1-3, or 1-2 nucleotides apart.

In some embodiments of the method for determining a location of a target nucleic acid in a biological sample described herein, each first probe oligonucleotide of the plurality is extended with an engineered family B polymerase described herein. In some embodiments, the engineered family B polymerase comprises an amino acid sequence having: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 10, 20-22, or 31; (b) at least 95% identity to the amino acid sequence of SEQ ID NO: 1, 10, 20-22, or 31; (c) at least 97% identity to the amino acid sequence of SEQ ID NO: 1, 10, 20-22, or 31; (d) at least about 10, at least about 15, at least about 16, at least about 18, at least about 20, at least about 25, or at least about 30 substitutions in the amino acid sequence of SEQ ID NO: 1; (e) at least 97% identity to the amino acid sequence of SEQ ID NO: 1, 10, 20-22, or 31 and at least about 16 substitutions in the amino acid sequence of SEQ ID NO: 1, 6, 8, 10, 20-22, or 31; or (f) 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 10, 20-22, or 31. In some embodiments, the engineered non-strand displacing reverse RT comprises an amino acid sequence selected from SEQ ID NO: 2-5, 11,12, and 25-30.

In some embodiments of the method for determining a location of a target nucleic acid in a biological sample described herein, the method further comprises extending a 3′ end of the capture probe using the ligation product. In some embodiments, extending the 3′ end of the capture probe comprises reverse transcribing the target nucleic acid using an engineered family B polymerase described herein.

In some embodiments of the method for determining a location of a target nucleic acid in a biological sample described herein, the determining step (i) comprises amplifying all or part of the ligation product using an engineered family B polymerase described herein. In some embodiments, the amplified product comprises (i) all or part of sequence of the ligation product, or a complement thereof, and (ii) the sequence of the spatial barcode, or a complement thereof.

Another aspect of the present disclosure provides a method of analyzing a sample, where the sample is a biological sample, where optionally in some embodiments it is a Formalin-Fixed Paraffin-Embedded (FFPE) sample, a formalin-fixed sample, a paraffin-embedded sample, a frozen sample, or a fresh sample; a single cell and/or a nucleus from a plurality of cells or nuclei, for example in a suspension and/or from homogenized tissues, which cells and/or nuclei are fresh, frozen, permeabilized, and/or fixed by any suitable fixative, including PFA, and/or (c) a tissue slice which is fresh, frozen, FFPE, formalin-fixed, paraffin embedded or in any other suitable form, comprising a nucleic acid molecule, comprising, consisting essentially of, or consisting of: (a) providing: (i) a sample comprising the nucleic acid molecule, where the nucleic acid molecule comprises a first target region and a second target region, where optionally in some embodiments the first target region is adjacent to the second target region; (ii) a first probe comprising a first probe sequence, and optionally another probe sequence, where the first probe sequence of the first probe is complementary to the first target region of the nucleic acid molecule, and where the first probe sequence comprises a first reactive moiety; and (iii) a second probe comprising a second probe sequence, where the second probe sequence of the second probe is complementary to the second target region of the nucleic acid molecule, and where the second probe sequence comprises a second reactive moiety; (b) subjecting the sample to conditions sufficient to (i) hybridize the first probe sequence of the first probe to the first target region of the nucleic acid molecule, and (ii) hybridize the second probe sequence of the second probe to the second target region of the nucleic acid molecule, such that the first reactive moiety of the first probe sequence of the first probe is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides. Alternatively, the first reactive moiety of the first probe sequence of the first probe is separated by about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides, or by 1-1400, 1-1300, 1-1200, 1-1100, by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe; (c) subjecting the first reactive moiety and the second reactive moiety to conditions sufficient to yield a probe-linked nucleic acid molecule comprising the first probe linked to the second probe; where in non-limiting embodiments, suitable conditions include contacting the first reactive moiety and the second reactive moiety with a ligase; and optionally (d) barcoding the probe-linked nucleic acid molecule to generate a barcoded probe-linked nucleic acid.

In some embodiments of the methods of analyzing a sample comprising a nucleic acid molecule, the method determines the presence or absence of a genetic variant in a nucleic acid, where in some embodiments the variant is detected in a nucleic acid in or from a single cell, and/or optionally at a spatial location in the biological sample. In some embodiments, the method determines the location of a genetic variant in a target nucleic acid in the biological sample. In some embodiments, the method comprises RNA-templated ligation.

In some embodiments, the methods of analyzing a sample comprising a nucleic acid molecule further comprise additional steps of nucleic acid processing to generate sequencing library from the barcoded probe-linked nucleic acids, determining sequences from the sequencing library, and correlating determined sequences with specific samples and/or partitions.

In some embodiments of the methods of analyzing a sample comprising a nucleic acid molecule, the probe-linked nucleic acid molecule of step (d) is in a partition, and under suitable conditions, where the partition comprises a partition specific barcode to generate a barcoded probe-linked nucleic acid molecule, and where, the partition is a single cell and/or a single nucleus, the partition comprises a single cell, single nucleus, nucleic acids from a single cell and/or single cell nucleus, or a combination of single cells, single nuclei, and/or nucleic acids from these.

In some herein, where a partition comprises multiple cells or nuclei, e.g., in multiplexing methods, the cells and/or nuclei comprise any suitable sequence, e.g., a barcode and/or index, that permits computationally identifying nucleic acid(s) that originated from a single cell and/or nucleus. In some embodiments, one or both of the probes comprise additional sequences, including without limitation probe specific barcode sequence(s), UMI, and any further sequences for nucleic acid processing, and sequencing library generation.

In some embodiments of the method of analyzing a sample comprising a nucleic acid molecule, steps (a), (b) and (c) are conducted in bulk; or (a) and (b) are conducted in bulk, and (c) is conducted in a partition.

In some embodiments of the method of analyzing a sample comprising a nucleic acid molecule described herein, when the first probe is separated by more than zero nucleotides, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides from the second probe; or by about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides, or by 1-1400, 1-1300, 1-1200, 1-1100, by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second probe, the method further comprises contacting an engineered family B polymerase of the disclosure, e.g. without limitation of Pfuengineered family B polymerases, Targengineered family B polymerases, with nucleic acid molecules from step (b) comprising hybridized probe and target sequences, under suitable conditions to permit extension from the first probe, to produce a polymerized nucleic acid product extending to the second probe, i.e., a gap fill reaction between the first and second probe in the presence of one of the engineered family B polymerases of the disclosure. In some embodiments, the enzyme is any of the engineered family B polymerases described herein. In some embodiments, the engineered family B polymerase comprises the amino acid sequence set forth in SEQ ID NO: 2-5, 11, 12, and 25-30 or an amino acid sequence having at least 90% sequence identity to the amino acid sequence set forth in SEQ ID NO: 2-5, 11, 12, and 25-30.

In some embodiments of the methods of analyzing a sample comprising a nucleic acid molecule, the gap fill reaction is conducted in bulk and/or in a partition. In certain embodiments, the partition is a droplet, a well, a cell and/or a nucleus. In certain embodiments, the cell and/or nucleus is fixed.

Another aspect of the present disclosure provides a method for analyzing a target nucleic acid in a biological sample, the method comprising, consisting of, or consisting essentially of: (a) contacting the biological sample with a first probe comprising a first probe sequence, and optionally another probe sequence, where the first probe sequence of the first probe is complementary to a first target region of the nucleic acid molecule, and where the first probe sequence comprises a first reactive moiety; and (iii) a second probe comprising a second probe sequence, where the second probe sequence of the second probe is complementary to a second target region of the nucleic acid molecule, and where the second probe sequence comprises a second reactive moiety; (b) hybridizing the plurality of first probe oligonucleotide to the first target region and the plurality of second probe oligonucleotide to the second target region, such that the first reactive moiety of the first probe sequence of the first probe is separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides, by about: 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides, or by 1-1400, 1-1300, 1-1200, 1-1100, 1-1000, 1-900, 1-800, 1-700, 1-600, 1-500, 1-400, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe; (c) subjecting the first reactive moiety and the second reactive moiety to conditions sufficient to yield a probe-linked nucleic acid molecule comprising the first probe linked to the second probe; in non-limiting embodiments, suitable conditions include contacting the first reactive moiety and the second reactive moiety with a ligase; (d) (optionally) releasing the probe-linked nucleic acid molecule from the target nucleic acid; (e) contacting the probe-linked nucleic acid molecule with a substrate comprising a plurality of capture probes, to hybridize the probe-linked nucleic acid molecule to a capture domain of the capture probe which is affixed to the substrate; (f) further processing the hybridized probe-linked nucleic acid molecule(s) to generate a sequencing library, (g) determining sequences of the probe-linked nucleic acid molecules in the sequencing library or a complement thereof, and (h) using the determined sequence(s) identifying the location of the in the biological sample.

In some embodiments, of this method, the plurality of first probe oligonucleotides and the plurality of second probe oligonucleotides target a plurality of nucleic acids in the biological sample. In some embodiments, each first probe and each second probe of the plurality comprise sequences are substantially complementary to a target nucleic acid in the biological sample, and each second probe of the plurality comprises a capture probe domain sequence. In some embodiments, the sample is fixed to a solid support, e.g., a slide, and method determines spatial position of the target nucleic acids in the sample.

In some embodiments, when the first probe is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides, the method further comprises a gap fill reaction in the presence of one of the engineered family B polymerases of the disclosure.

In some embodiments, the enzyme is any of the engineered family B polymerases described herein. In some embodiments, the engineered family B polymerase comprises the amino acid sequence set forth in SEQ ID NO: 2-5, 11, 12, and 25-30 or an amino acid sequence having at least 90% sequence identity to the amino acid sequence set forth in SEQ ID NO: 2-5, 11, 12, and 25-30. In some embodiments of the method for analyzing a target nucleic acid in a biological sample described herein, one or both of the probes comprise additional sequences, including without limitation probe specific barcode sequence(s), UMI, and any further sequences for nucleic acid processing, and sequencing library generation.

One aspect of the present disclosure provides a method of using an engineered family B polymerase (e.g., engineered recombinant Family-B polymerases; engineered DNA polymerase enzymes; engineered DNA polymerases) that has reverse transcriptase activity and substantially lacks strand displacement activity, the method comprising, consisting essentially of, or consisting of, contacting the engineered family B polymerase with a plurality of nucleic acid templates under suitable conditions to produce a polymerized nucleic acid product, where the plurality of nucleic acid templates comprises a plurality of RNAs, DNAs, or nucleic acids comprising an unnatural nucleotide; where the engineered family B polymerase comprises an amino acid sequence having at least 75% sequence identity to the amino acid sequence of Pyrococcus furiosus (pfu) polymerase (SEQ ID NO: 1), Thermococcus gorgonarius polymerase (Tgo polymerase) (SEQ ID NO: 10), Thermococcus kodakarensis (KOD1) polymerase (SEQ ID NO: 6 or 8), Thermococcus litoralis (VENT®) polymerase (SEQ ID NO: 20), Pyrococcus sp. (Deep Vent)polymerase (SEQ ID NO: 21), Thermococcus sp. (9°N) polymerase (SEQ ID NO: 22), or Thermococcus argininiproducens (Targ) polymerase (SEQ ID NO: 31). Optionally, the engineered family B polymerase has no detectable strand displacement activity.

In some embodiments, the nucleic acid template comprises a first probe and a second probe which are hybridized to a first and a second target nucleic acids/target regions, optionally, the second target nucleic acid/target region is a mRNA.

In some embodiments, (a) the first probe is operably linked to the second probe; or (b) the first probe and the second probe are part of the same molecule; or (c) the first probe and the second probe are part of different molecules.

In some embodiments, the first probe hybridized to the first target sequence and the second probe hybridized to the second target sequence are not immediately adjacent to each other. Optionally, there is more than 1 nucleotide between the first and the second target sequences and/or the first and the second probes.

In some embodiments, the polymerized product is generated between the first probe hybridized to the first target sequence/target region and the second probe hybridized to the second target sequence/target region.

In some embodiments, the first and the second target sequences and/or the first and the second probes hybridized to the first and the second target sequences are separated by: (a) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or (b) 1-1000, 1-750, 1-500, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides.

In some embodiments, the nucleic acid templates in the plurality of nucleic acid templates are located in a biological sample. In some embodiments, the biological sample comprises: (a) a Formalin-Fixed Paraffin-Embedded (FFPE) sample, a formalin-fixed sample, a paraffin-embedded sample, a frozen sample, or a fresh sample; (b) a single cell; and/or (c) a tissue.

In some embodiments of the method described herein, the method determines the presence of a genetic variant in a nucleic acid. Optionally, the variant is at a spatial location in the biological sample. In some embodiments, the method determines the location of a genetic variant in a target nucleic acid in the biological sample. In some embodiments, the method comprises RNA-templated ligation.

Another aspect of the present disclosure provides a nucleic acid extension method comprising: (a) contacting a target nucleic acid molecule with an engineered family B polymerase (e.g., engineered recombinant Family-B polymerases; engineered DNA polymerase enzymes; engineered DNA polymerases) that has reverse transcriptase activity and substantially lacks strand displacement activity and a plurality of nucleic acid barcoded molecules comprising a barcode sequence, and (b) incubating the target nucleic acid, the engineered the engineered family B polymerase, and the plurality of nucleic acid barcoded molecules under conditions in which the plurality of nucleic acid barcoded molecules are extended by the engineered family B polymerase;

where: (i) the engineered family B polymerase comprises an amino acid sequence that has at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31; (ii) one of the plurality of nucleic acid barcoded molecules hybridizes to the target nucleic acid molecule; and (iii) the engineered family B polymerase extends the one of the plurality of nucleic acid barcoded molecules that is hybridized to the target nucleic acid molecule.

In some embodiments, (a) the nucleic acid comprises a ribonucleic acid (RNA) molecule; and (b) the engineered family B polymerase reverse transcribes the RNA molecule into a complementary DNA, and then amplifies the complementary DNA into a nucleic acid product in the same reaction. In some embodiments, the RNA molecule comprises a messenger RNA (mRNA) molecule.

In some embodiments: (a) the plurality of nucleic acid barcoded molecules further comprises an oligo(dT) sequence; and (b) the engineered family B polymerase reverse transcribes the mRNA molecule into a complementary DNA (cDNA) molecule using the mRNA hybridized to the oligo(dT) sequence of the plurality of nucleic acid barcoded molecules as a template, thereby generating a complementary DNA molecule comprising the barcode sequence. In that embodiment, the engineered family B polymerase further amplifies the complementary DNA molecule comprising the barcode sequence, thereby generating an amplified DNA product comprising the barcode sequence or complements thereof.

In some embodiments of the nucleic acid method described herein, the method further comprises a second nucleic acid molecule comprising an oligo(dT) sequence; the plurality of nucleic acid barcoded molecules further comprises an oligo(dT) sequence; and the engineered family B polymerase reverse transcribes the mRNA molecule using the second nucleic acid molecule comprising the oligo(dT) sequence, thereby generating a complementary DNA molecule. In that embodiment, the engineered family B polymerase further amplifies the complementary DNA molecule using the plurality of nucleic acid barcoded molecules, thereby generating an amplified DNA product comprising a barcode sequence.

In some embodiments of the nucleic acid extension method described herein the plurality of nucleic acid barcoded molecules is attached to a support; and optionally, the support is selected from the group consisting of an array, a bead, a gel bead, a microparticle, and a polymer.

In some embodiments of the nucleic acid extension method described herein, the engineered family B polymerase comprises the amino acid sequence set forth in SEQ ID NOs: 2-5, 11, 12, and 25-30 or an amino acid sequence having at least 90% sequence identity to the amino acid sequence set forth in SEQ ID NO: 2-5, 11, 12, and 25-30.

Another aspect of the present disclosure provides a method for determining a location of a target nucleic acid in a biological sample, the method comprising: (a) contacting the biological sample with a plurality of first probe oligonucleotides and a plurality of second probe oligonucleotides, where: (i) the plurality of first probe oligonucleotides and the plurality of second probe oligonucleotides target a plurality of nucleic acids in the biological sample, (ii) each first probe and each second probe of the plurality of oligonucleotides comprise sequences that are substantially complementary to a target nucleic acid in the biological sample, and (iii) each second probe of the plurality of oligonucleotides comprises a capture probe domain sequence; (b) hybridizing the plurality of first probe oligonucleotides and the plurality of second probe oligonucleotides to the target nucleic, where each first probe oligonucleotide and each second probe oligonucleotide of the plurality hybridize to sequences that are not adjacent to each other on the plurality of target nucleic acids, optionally each first probe and each second probe of the oligonucleotide of the plurality are part of the same molecule or are part of different molecules; (c) extending each first probe oligonucleotide of the plurality using an engineered family B polymerase (e.g., engineered recombinant Family-B polymerases; engineered DNA polymerase enzymes; engineered DNA polymerases) that has reverse transcriptase activity and substantially lacks strand displacement activity to generate an extended first probe oligonucleotide of the plurality, thereby filling in a gap between the first probe oligonucleotide and the second probe oligonucleotide of the plurality; the engineered family B polymerase comprises an amino acid sequence having at least 75% sequence identity to the amino acid sequence of Pyrococcus furiosus (pfu) polymerase (SEQ ID NO: 1), Thermococcus gorgonarius polymerase (Tgo polymerase) (SEQ ID NO: 10), Thermococcus kodakarensis (KOD1) polymerase (SEQ ID NO: 6 or 8), Thermococcus litoralis (VENT®) polymerase (SEQ ID NO: 20), Pyrococcus sp. (Deep Vent)polymerase (SEQ ID NO: 21), Thermococcus sp. (9°N) polymerase (SEQ ID NO: 22), or Thermococcus argininiproducens (Targ) polymerase (SEQ ID NO: 31), optionally the engineered family B polymerase has no detectable strand displacement activity; (d) cleaving the sequence of non-complementary nucleotides; (e) ligating the extended first probe oligonucleotide and the second probe oligonucleotide of the plurality, thereby creating a ligated probe that is substantially complementary to the target nucleic acid; (f) releasing the ligated probe from the target nucleic acid; (g) contacting the biological sample with a substrate comprising a plurality of capture probes, where each capture probe of the plurality of capture probes comprises: (i) a spatial barcode and (ii) a capture domain and where the capture domain comprises a sequence that is complementary to all or a portion of the capture probe domain of the second probe oligonucleotide; (h) hybridizing the ligation product to the capture domain of the capture probe affixed to the substrate; and (i) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the location of the analyte in the biological sample.

In that embodiment, the generating the ligation product comprises ligating the extended first probe to the second probe of the plurality using an enzymatic ligation or a chemical ligation, optionally the enzymatic ligation utilizes a ligase.

In some embodiments of the method for determining a location of a target nucleic acid in a biological sample described herein, each first probe oligonucleotide of the plurality and each second probe oligonucleotide of the plurality hybridized to nucleic acid sequences that are: (a) about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other; or (b) 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900, or 1000 nucleotides away from each other.

In that embodiment, each first probe oligonucleotide of the plurality and each second probe oligonucleotide of the plurality hybridized to nucleic acid sequences that are: (a) at least about 1-100, at least about 1-90, at least about 1-80, at least about 1-70, at least about 1-60, at least about 1-50, at least about 1-40, at least about 1-30, at least about 1-20, at least about 1-10, at least about 1-9, at least about 1-8, at least about 1-7, at least about 1-6, at least about 1-5, at least about 1-4, at least about 1-3, at least about 1-2 nucleotides apart; or (b) 1-100, 1-90, 1-80, 1-70, 1-60, 1-50, 1-40, 1-30, 1-20, 1-10, 1-9, 1-8, 1-7, 1-6, 1-5, 1-4, 1-3, or 1-2 nucleotides apart.

In some embodiments, each first probe oligonucleotide of the plurality is extended with an engineered family B polymerase, optionally the engineered family B polymerase comprises an amino acid sequence having at least 75% sequence identity to the amino acid sequence of Thermococcus gorgonarius polymerase (Tgo polymerase) or SEQ ID NO: 10.

In some embodiments of the method for determining a location of a target nucleic acid in a biological sample described herein, each first probe oligonucleotide of the plurality and each second probe oligonucleotide of the plurality are operably linked; or each first probe oligonucleotide of the plurality and each second probe oligonucleotide of the plurality are part of the same molecule.

In some embodiments of the method for determining a location of a target nucleic acid in a biological sample described herein, the engineered family B polymerase comprises an amino acid sequence having: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31; (b) at least 95% identity to the amino acid sequence of SEQ ID NO: 1, 6, 8, 10, 20-22, or 31; (c) at least 97% identity to the amino acid sequence of SEQ ID NO: 1, 6, 8, 10, 20-22, or 31; (d) at least about 10, at least about 15, at least about 16, at least about 18, at least about 20, at least about 25, or at least about 30 substitutions in the amino acid sequence of SEQ ID NO: 1; (e) at least 97% identity to the amino acid sequence of SEQ ID NO: 1, 6, 8, 10, 20-22, or 31 and at least about 16 substitutions in the amino acid sequence of SEQ ID NO: 1, 6, 8, 10, 20-22, or 31; or (f) 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 6, 8, 10, 20-22, or 31.

In some embodiments, the engineered family B polymerase comprises an amino acid sequence selected from SEQ ID NO: 2-5, 11, 12, and 25-30; optionally the engineered family B polymerase is an engineered non-strand displacing reverse RT.

In some embodiments of the method for determining a location of a target nucleic acid in a biological sample described herein, the method further comprises, consists of or consists essentially of extending a 3′ end of the capture probe using the ligation product. In that embodiment, extending the 3′ end of the capture probe comprises reverse transcribing the target nucleic acid using the engineered family B polymerase.

In some embodiments of the method for determining a location of a target nucleic acid in a biological sample described herein, the determining step (i) comprises amplifying all or part of the ligation product using the engineered family B polymerase.

In that embodiment, the amplified product comprises (i) all or part of sequence of the ligation product, or a complement thereof, and (ii) the sequence of the spatial barcode, or a complement thereof.

Another aspect of the present disclosure provides a method of analyzing a sample comprising a nucleic acid molecule, the method comprising: (a) providing: (i) a sample comprising the nucleic acid molecule, where the nucleic acid molecule comprises a first target region and a second target region, optionally the first target region is adjacent to the second target region; (ii) a first probe comprising a first probe sequence, and optionally another probe sequence, where the first probe sequence of the first probe is complementary to the first target region of the nucleic acid molecule, and where the first probe sequence comprises a first reactive moiety; and (iii) a second probe comprising a second probe sequence, where the second probe sequence of the second probe is complementary to the second target region of the nucleic acid molecule, and where the second probe sequence comprises a second reactive moiety; (b) subjecting the sample to conditions sufficient to (i) hybridize the first probe sequence of the first probe to the first target region of the nucleic acid molecule, and (ii) hybridize the second probe sequence of the second probe to the second target region of the nucleic acid molecule, such that the first reactive moiety of the first probe sequence of the first probe is separated by (a) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides; or (b) by 1-1000, 1-900, 1-800, 1-700, 1-600, 1-500, 1-400, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe, optionally the first probe and the second probe are part of the same molecule or are part of different molecules; (c) subjecting the first reactive moiety and the second reactive moiety to conditions sufficient to yield a probe-linked nucleic acid molecule comprising the first probe linked to the second probe; where suitable conditions include contacting the first reactive moiety and the second reactive moiety with a ligase; and (d) barcoding the probe-linked nucleic acid molecule to generate a barcoded probe-linked nucleic acid; (e) optionally processing the nucleic acid to generate sequencing library from the barcoded probe-linked nucleic acids, (f) determining sequences from the sequencing library, and (g) correlating determined sequences with specific samples and/or partitions.

In some embodiments, in step (d), the probe-linked nucleic acid molecule is in a partition, optionally the partition comprises a partition specific barcode to generate a barcoded probe-linked nucleic acid molecule, and the partition comprises a single cell, a single nucleus, nucleic acids from a single cell, single cell nuclei, or a combination thereof.

In some embodiments, where a partition comprises multiple cells, the cells comprise any suitable barcode and/or index that permits computationally identifying nucleic acids that originated from a single cell and/or nucleus.

In some embodiments of the method of analyzing a sample comprising a nucleic acid molecule described herein, the first probe, the second probe, or the first and second probes comprise additional sequences selected from probe specific barcode sequences, UMI, or any further sequences for nucleic acid processing and sequencing library generation.

In some embodiments, the steps (a), (b) and (c) are conducted in bulk. In some embodiments, steps (a) and (b) are conducted in bulk, and (c) is conducted in a partition.

In some embodiments, when the first probe is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe, the method further comprises a gap fill reaction in the presence of an engineered family B polymerase that has reverse transcriptase activity and substantially lacks strand displacement activity; optionally the engineered family B polymerase comprises an amino acid sequence having at least 75% sequence identity to the amino acid sequence of Pyrococcus furiosus (pfu) polymerase (SEQ ID NO: 1), Thermococcus gorgonarius polymerase (Tgo polymerase) (SEQ ID NO: 10), Thermococcus kodakarensis (KOD1) polymerase (SEQ ID NO: 6 or 8), Thermococcus litoralis (VENT®) polymerase (SEQ ID NO: 20), Pyrococcus sp. (Deep Vent)polymerase (SEQ ID NO: 21), Thermococcus sp. (9°N) polymerase (SEQ ID NO: 22), or Thermococcus argininiproducens (Targ) polymerase (SEQ ID NO: 31); and optionally wherein the engineered family B polymerase has no detectable strand displacement activity.

In that embodiment, the engineered family B polymerase comprises an amino acid sequence that has: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31; (b) at least 95% identity to the amino acid sequence of SEQ ID NO: 1, 6, 8, 10, 20-22, or 31; (c) at least 97% identity to the amino acid sequence of SEQ ID NO: 1, 6, 8, 10, 20-22, or 31; (d) at least about 10, at least about 15, at least about 16, at least about 18, at least about 20, at least about 25, or at least about 30 substitutions in the amino acid sequence of SEQ ID NO: 1; (e) at least 97% identity to the amino acid sequence of SEQ ID NO: 1, 6, 8, 10, 20-22, or 31 and at least about 16 substitutions in the amino acid sequence of SEQ ID NO: 1, 6, 8, 10, 20-22, or 31; or (f) 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 6, 8, 10, 20-22, or 31.

In that embodiment, the engineered family B polymerase comprises the amino acid sequence set forth in SEQ ID NO: 2-5, 11, 12, and 25-30 or an amino acid sequence having at least 90% sequence identity to the amino acid sequence set forth in SEQ ID NO: 2-5, 11, 12, and 25-30.

In some embodiments of the method of analyzing a sample comprising a nucleic acid molecule described herein, the gap fill reaction is conducted in bulk and/or a partition. In some embodiments, the partition is a droplet, a well, a cell and/or a nucleus. In some embodiments, the sample is fixed.

Another aspect of the present disclosure provides a method for analyzing a target nucleic acid in a biological sample, the method comprising: (a) contacting the biological sample with: (i) a first probe comprising a first probe sequence, and optionally another probe sequence, where the first probe sequence of the first probe is complementary to a first target region of the nucleic acid molecule, and where the first probe sequence comprises a first reactive moiety; and (ii) a second probe comprising a second probe sequence, where the second probe sequence of the second probe is complementary to a second target region of the nucleic acid molecule, and where the second probe sequence comprises a second reactive moiety; (b) hybridizing the first probe to the first target region and the second probe to the second target region, such that the first reactive moiety of the first probe sequence of the first probe is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe, optionally the first probe and the second probe are part of the same molecule or part of different molecule; (c) subjecting the first reactive moiety and the second reactive moiety to conditions sufficient to yield a probe-linked nucleic acid molecule comprising the first probe linked to the second probe; wherein suitable conditions include contacting the first reactive moiety and the second reactive moiety with a ligase; (d) optionally releasing the probe-linked nucleic acid molecule from the target nucleic acid; (e) contacting the probe-linked nucleic acid molecule with a substrate comprising a plurality of capture probes to hybridize the probe-linked nucleic acid molecule to a capture domain of the capture probe which is affixed to the substrate; (f) further processing the hybridized probe-linked nucleic acid molecule to generate a sequencing library; (g) determining sequences of the probe-linked nucleic acid molecules in the sequencing library or a complement thereof, and (h) using the determined sequences to identify the location of the target nucleic acid sequence in the biological sample.

In some embodiments, the plurality of first probe oligonucleotides and the plurality of second probe oligonucleotides target a plurality of nucleic acids in the biological sample.

In some embodiments, each first probe and each second probe comprise sequences that are substantially complementary to a target nucleic acid in the biological sample, and each second probe comprises a capture probe domain sequence.

In some embodiments of the method for analyzing a target nucleic acid in a biological sample described herein, the biological sample is fixed to a solid support, optionally the solid support is a slide, and the method determines spatial position of the target nucleic acids in the biological sample.

In some embodiments of the method for analyzing a target nucleic acid in a biological sample described herein, when the first reactive moiety of the first probe is separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides, or by 1-1000, 1-900, 1-800, 1-700, 1-600, 1-500, 1-400, 1-300 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides from the second reactive moiety of the second probe sequence of the second probe, the method further comprises a gap fill reaction in the presence of an engineered family B polymerase that has reverse transcriptase activity and substantially lacks strand displacement activity; optionally the engineered family B polymerase comprises an amino acid sequence having at least 75% sequence identity to the amino acid sequence of Pyrococcus furiosus (pfu) polymerase (SEQ ID NO: 1), Thermococcus gorgonarius polymerase (Tgo polymerase) (SEQ ID NO: 10), Thermococcus kodakarensis (KOD1) polymerase (SEQ ID NO: 6 or 8), Thermococcus litoralis (VENT®) polymerase (SEQ ID NO: 20), Pyrococcus sp. (Deep Vent)polymerase (SEQ ID NO: 21), Thermococcus sp. (9°N) polymerase (SEQ ID NO: 22), or Thermococcus argininiproducens (Targ) polymerase (SEQ ID NO: 31); and optionally the engineered family B polymerase has no detectable strand displacement activity.

In some embodiments of the method for analyzing a target nucleic acid in a biological sample described herein, the first probe, the second probe, or the first and second probes comprise additional sequences selected from probe specific barcode sequences, UMI, or any further sequences for nucleic acid processing and sequencing library generation

In some embodiments of the method for analyzing a target nucleic acid in a biological sample described herein, the engineered family B polymerase comprises an amino acid sequence that has: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 6, 8, 10, 20-22, or 31; (b) at least 95% identity to the amino acid sequence of SEQ ID NO: 1, 6, 8, 10, 20-22, or 31; (c) at least 97% identity to the amino acid sequence of SEQ ID NO: 1, 6, 8, 10, 20-22, or 31; (d) at least about 10, at least about 15, at least about 16, at least about 18, at least about 20, at least about 25, or at least about 30 substitutions in the amino acid sequence of SEQ ID NO: 1; (e) at least 97% identity to the amino acid sequence of SEQ ID NO: 1, 6, 8, 10, 20-22, or 31 and at least about 16 substitutions in the amino acid sequence of SEQ ID NO: 1, 6, 8, 10, 20-22, or 31; or (f)
80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 6, 8, 10, 20-22, or 31.

In some embodiments, the engineered family B polymerase comprises the amino acid sequence set forth in SEQ ID NO: 2-5, 11, 12, and 25-30; or an amino acid sequence having at least 90% sequence identity to the amino acid sequence set forth in SEQ ID NO: 2-5, 11, 12, and 25-30.

In some embodiments of any of the methods, the ligase comprises a family B ligase. In some embodiments, the ligase is selected from the group consisting of T4 DNA ligase, T4 RNA ligase, Chlorella virus DNA ligase, Paramecium bursaria Chlorella virus 1 DNA ligase I (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase. In some embodiments, the ligase comprises a single stranded DNA ligase, or an Archaeal RNA ligase.

One aspect of the present disclosure provides an engineered family B polymerase comprising an amino acid sequence having at least 75% sequence identity to the amino acid sequence of Pyrococcus furiosus (pfu) polymerase (SEQ ID NO: 1), Thermococcus gorgonarius polymerase (Tgo polymerase) (SEQ ID NO: 10), Thermococcus litoralis (VENT®) polymerase (SEQ ID NO: 20), Pyrococcus sp. (Deep Vent) polymerase (SEQ ID NO: 21), Thermococcus sp. (9°N) polymerase (SEQ ID NO: 22), or Thermococcus argininiproducens (Targ) polymerase (SEQ ID NO: 31), where the engineered family B polymerase has reverse transcriptase activity and substantially lacks strand displacement activity, optionally where the enzyme has no detectable strand displacement activity. In some embodiments, the engineered family B polymerase has DNA and RNA polymerase activity.

In certain embodiments, the engineered family B polymerase substantially lacks strand displacement activity. In some embodiments, the engineered family B polymerase displaces no more than 10, 9, 8, 7, 6, 5, 4, 3, 2, or 1 nucleotides; 1-2, 1-3, 1-4, 1-5, 1-6, 1-7, 1-8, 1-9, 1-10 nucleotides; 1, 2, 3, 4, 5, 6, 7, 8, 9 or 10 nucleotides; about 6 nucleotides; or about 10 nucleotides.

In certain embodiments, the engineered family B polymerase is not KOD-RTX. In certain embodiments, the engineered family B polymerase does not comprise SEQ ID NO: 7.

In some embodiments of the engineered family B polymerase disclosed herein, the engineered family B polymerase comprises an amino acid sequence that has: (a) at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 1, 10, 20-22, or 31; (b) at least 95% identity to the amino acid sequence of SEQ ID NO: 1, 10, 20-22, or 31; (c) at least 97% identity to the amino acid sequence of SEQ ID NO: 1, 10, 20-22, or 31; (d) at least about 10, at least about 15, at least about 16, at least about 18, at least about 20, at least about 25, or at least about 30 substitutions in the amino acid sequence of SEQ ID NO: 1; (e) at least 97% identity to the amino acid sequence of SEQ ID NO: 1, 10, 20-22, or 31 and at least about 16 substitutions in the amino acid sequence of SEQ ID NO: 1, 10, 20-22, or 31; or (f) 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1, 10, 20-22, or 31. In that embodiment, the engineered family B polymerase is not KOD-RT. In that embodiment, the engineered family B polymerase is a Tgo-RT.

In some embodiments of the engineered family B polymerase disclosed herein, the engineered family B polymerase comprises an amino acid sequence having 90%, 91%, 92%, 93%, 94%, 95%, 96%, 97%, 98%, or 99% sequence identity to the amino acid sequence of SEQ ID NO: 1.

In some embodiments, the engineered family B polymerase comprises: (a) an amino acid substitution at position I38, R97, K118, I137, R382, Y385, V390, K467, F494, T515, I522, F588, E665, S712, N736, W769, or any combination thereof, or the combination of all substitutions in SEQ ID NO: 1; optionally the substitution is I38L, R97M, K118I, I137L, R382H, Y385H, V390I, K467R, F494L, T515I, I522L, F588L, E665K, S712V, N736K, or W769R in SEQ ID NO: 1, or any combination thereof, or the combination of all substitutions; or (b) any amino acid substitution selected from 38L, 97M, 118I, 137L, 381H, 384H, 389I, 466R, 493L, 514I, 521L, 587L, 664K, 711V, 735K, 768R, or any combination thereof, or the combination of all substitutions from SEQ ID NO: 7

In some embodiments, the engineered family B polymerase further comprises: (a) an amino acid substitution at a position corresponding to any one of position I2, V93, D141, E143, or A485 in SEQ ID NO: 7, or any combination thereof, or the combination of all substitutions, optionally the substitutions are I2V, V93Q, D141A, E143A, A485L, or any combination thereof, or the combination thereof from SEQ ID NO: 7; or (b) an amino acid substitution at position I2, V93, D141, E143, or A486 in SEQ ID NO: 1, any combination thereof, or the combination of all substitutions in SEQ ID NO: 1, optionally the substitution is I2V, V93Q, D141A, E143A, or A486L in SEQ ID NO: 1, any combination thereof, or the combination of all substitutions in SEQ ID NO: 1.

In some embodiments, the engineered family B polymerase comprises: (a) an aspartic acid substitution at position 141 (optionally in certain embodiments D141A); (b) a glutamic acid substitution at position 143 (optionally in certain embodiments E143A); (c) an alanine substitution at position 485 (optionally in certain embodiments A485L); (d) a valine substitution at position 93 (optionally in certain embodiments V93Q); (e) an arginine substitution at position 97 (in certain embodiments R97M); (f) a tyrosine substitution at position 384 (optionally in certain embodiments Y384H); (g) a valine substitution at position 389 (optionally in certain embodiments V389I); (h) a phenylalanine substitution at position 494 (optionally in certain embodiments F494L); (i) a phenylalanine substitution at position 588 (optionally in certain embodiments F588L); (j) a glutamic acid substitution at position 665 (optionally in certain embodiments E665K); (k) a serine substitution at position (optionally in certain embodiments S712V); (l) a tryptophan substitution at position 769 (optionally in certain embodiments W769R); (m) an isoleucine substitution at position 2 (optionally in certain embodiments I2V); (n) an isoleucine to leucine substitution at position 38 (optionally in certain embodiments I38L); (o) a lysine substitution at position 118 (optionally in certain embodiments K118I); (p) a isoleucine substitution at position 137 (optionally in certain embodiments I137L); (q) an arginine to histidine substitution at position 381 (optionally in certain embodiments R381H); (r) a lysine to arginine substitution at position 466 (optionally in certain embodiments K466R); (s) a tyrosine to isoleucine substitution at position 514 (optionally in certain embodiments T514I); (t) an isoleucine to leucine substitution at position 521 (optionally in certain embodiments I521L); and/or (u) an asparagine to lysine substitution at position 735 (optionally in certain embodiments N735K) in SEQ ID NO: 1.

In some embodiments, the engineered family B polymerase: (a) comprises a substitution at positions 141 and/or 143 of SEQ ID NO: 1; or (b) comprises a substitution at position 141 of SEQ ID NO: 1; and (c) lacks proofreading activity.

In some embodiments, the engineered family B polymerase comprises: (a) R97M, D141A, E143A, Y385H, V393I, Y494L, F588L, E665K, S712V, and W769R substitutions in SEQ ID NO: 1; (b) I2V, I38L, R97M, K118I, I137L, E143A, R382H; Y385H, V390I, K465R, F494L, T515I, I522L, F588L, E665K, S712V, N736K, and W769R substitutions in SEQ ID NO: 1; (c) I2V, I38L, R97M, K118I, I137L, D141A, E143A, R382H, Y385H, V390I, K465R, F494L, T515I, I522L, F588L, E665K, S712V, N736K, and W769R substitutions in SEQ ID NO: 1; or (d) I2V, I38L, V93Q, R97M, K118I, I137L, D141A, E143A, R382H, Y385H, A486L, V390I, K467R, F494L, T515I, I522L, F588L, E665K, S712V, N736K, and W769R substitutions in SEQ ID NO: 1.

In some embodiments, the engineered family B polymerase comprises, consists substantially of, or consists of the amino acid sequence of SEQ ID NO: 2, 3, 4, 5, 10, 11, 12, 26, or 27.

In some embodiments of the engineered family B polymerase disclosed herein, the enzyme comprises, consists essentially of or consists of: (a) a substitution at a position corresponding to a position selected from 38, 97, 118, 137, 381, 384, 389I, 466, 493, 514, 521, 587, 664, 711, 735, or 768 in SEQ ID NO: 7, or any combination thereof, or the combination of all substitutions; or (b) any an amino acid substitution selected from 38L, 97M, 118I, 137L, 381H, 384H, 389I, 466R, 493L, 514I, 521L, 587L, 664K, 711V, 735K, 768R in SEQ ID NO: 7, or any combination thereof, or the combination of all substitutions. In that embodiment, the engineered family B polymerase is not KOD-RT.

In some embodiments, the engineered family B polymerase further comprises any amino acid substitution at a position corresponding to position I2, V93, D141, E143, A485 in SEQ ID NO: 7, or any combination thereof, or the combination of all substitutions. Optionally the substitutions are I2V, V93Q, D141A, E143A, A485L, or any combination thereof, or the combination thereof in SEQ ID NO: 7. In that embodiment, the engineered family B polymerase is not KOD-RT.

In some embodiments, the engineered family B polymerase is an engineered Thermococcus gorgonarius polymerase (Tgo polymerase). In some embodiments, the engineered family B polymerase comprises an amino acid sequence that that has at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 10. In some embodiments, the engineered family B polymerase comprises an amino acid sequence that that has at least 80%, at least 90%, at least 91%, at least 92%, at least 93%, at least 94%, at least 95%, at least 96%, at least 97%, at least 98%, or at least 99% sequence identity to the amino acid sequence of SEQ ID NO: 11 or 12.

In some embodiments, the engineered family B polymerase comprises the amino acid sequence of SEQ ID NO: 11, 12, 25, or 28-30.

In some embodiments, the engineered family B polymerase binds a DNA, an RNA, or a DNA-RNA hybrid complex. In some embodiments, the DNA-RNA hybrid is continuous or discontinuous.

In some embodiments of the engineered family B polymerase disclosed herein, the engineered family B polymerase further comprises a tag protein selected from the group consisting of an affinity tag, a fluorescent tag, or an expression and/or solubility enhancement tag.

In some embodiments, the tag is selected from hexahistidine tag (his-tag), small ubiquitin-like modifier tag (SUMO), a short peptide C-terminal tag, Thioredoxin (Trx) tag, a VariFlex™ C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), Flag tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the engineered family B polymerase comprises: (a) an hexahistidine tag (his-tag); or (b) an amino acid sequence of SEQ ID NO: 13; or an amino acid sequence having at least 90% sequence identity to the amino acid sequence of SEQ ID NO: 13.

In some embodiments, the engineered family B polymerase comprises a solubility enhancer tag selected from the group consisting of a SUMO tag, a GST tag, a Trx tag, a VariFlex™ C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments, the engineered family B polymerase comprises: (a) a short peptide C-terminal tag; (b) an amino acid sequence of SEQ ID NO: 14; or (c) an amino acid sequence having at least 90% sequence identity to the amino acid sequence of SEQ ID NO: 14.

In some embodiments, the tag further comprises: (a) an endoprotein cleavage sequence; (b) a cleavage sequence recognized by an endoprotein selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase IL, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase; or (c) an endoprotein cleavage sequence comprising the amino acid sequence of SEQ ID NO: 15, SEQ ID NO: 16, SEQ ID NO: 17, SEQ ID NO: 18, or SEQ ID NO: 19.

In some embodiments of the engineered family B polymerase disclosed herein, the enzyme reverse transcribes a RNA molecule having: (a) at least about 6, at least about 7, at least about 8, at least about 9, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 100, at least about 200, at least about 300, at least about 400, at least about 500, at least about 600, at least about 700, at least about 800, at least about 900, or at least about 1000 nucleotides; (b) 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 200, 300, 400, 500, 600, 700, 800, 900, or 1000 nucleotides; or (c) about: 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides.

In some embodiments, the enzyme reverse transcribes a RNA molecule: (a) that is at least about 1-1000, at least about 1-750, at least about 1-500, at least about 1-300, at least about 1-200, at least about 1-100, at least about 1-90, at least about 1-80, at least about 1-70, at least about 1-60, at least about 1-50, at least about 1-40, at least about 1-30, at least about 1-20, at least about 1-10, at least about 1-9, at least about 1-8, at least about 1-7, at least about 1-6, at least about 1-5, at least about 1-4, at least about 1-3, or at least about 1-2 nucleotides; or (b) that is 1-1400, 1-1300, 1-1200, 1-1100, 1-1000, 1-750, 1-500, 1-300, 1-200, 1-100, 1-90, 1-80, 1-70, 1-60, 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 nucleotides; (c) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides; or (d) about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1350, or 1400 nucleotides.

One aspect of the present disclosure provides an isolated nucleic acid molecule encoding an engineered family B polymerase described herein.

Another aspect of the present disclosure provides an expression vector comprising an isolated nucleic acid molecule encoding an engineered family B polymerase described herein.

Another aspect of the present disclosure comprises a host cell transfected with the expression vector comprising a nucleic acid molecule encoding an engineered family B polymerase described herein.

Another aspect of the present disclosure provides a method of using an engineered family B polymerase described herein, the method comprising, consisting essentially of or consisting of contacting the engineered family B polymerase with a plurality of nucleic acid templates under suitable conditions to produce a polymerized nucleic acid product, where the plurality of nucleic acid templates is a plurality of RNAs, DNAs, or nucleic acids comprising an unnatural nucleotide.

Another aspect of the present disclosure provides a kit comprising an engineered family B polymerase described herein. In some embodiments, the kit further comprises one or more of a vector, a nucleotide, a buffer, dNTPs, a ligase, a salt, and/or instructions.

Another aspect of the present disclosure provides a composition comprising an engineered family B polymerase described herein, a nucleic acid and at least one reagent for carrying out a reaction with a plurality of nucleic acid templates under suitable conditions to produce a polymerized nucleic acid product.

### IV. Kits

One aspect of the present disclosure provides a kit comprising the engineered enzyme or a derivative thereof as described herein. In some embodiments, the kit further comprises one or more of a vector, a nucleotide, a buffer, a salt, dNTPs, a ligase, and/or instructions. In another embodiment, a kit may comprise an engineered family B polymerase or a derivative thereof for use in reverse transcription or amplification of a nucleic acid molecule. In yet another embodiment, a kit may be used for single cell profiling of the transcriptome. In yet another embodiment, a kit may be used for spatial transcriptomics methods and assays. In yet another embodiment, a kit may be used for in situ methods and assays.

The kit may include suitable reaction buffers, dNTPs, one or more primers, one or more control reagents, or any other reagents disclosed for performing the methods of the present disclosure. The engineered family B polymerase or a derivative thereof, reaction buffer, and dNTPs may be provided separately or may be provided together in a master mix solution. When the engineered family B polymerase or a derivative thereof, reaction buffer, and dNTPs are provided in a master mix, the master mix is present at a concentration at least two times the working concentration indicated in instructions for use in an extension reaction. In other cases, the master mix may be present at a concentration at least three times, at least four times, at least five times, at least six times, at least seven times, at least eight times, at least nine times, or at least ten times, the working concentration indicated. The primer in the kits may be a poly-dT primer, a random N-mer primer, or a target-specific primer.

The kits may further include one, two, three, four, five or more, up to all of partitioning fluids, including both aqueous buffers and non-aqueous partitioning fluids or oils, nucleic acid barcode capture probes that are releasably associated with beads, as described herein, microfluidic devices, reagents for disrupting cells, reagents for amplifying nucleic acids, as well as instructions for using any of the foregoing in the methods described herein. The kit may comprise a ligase. The ligase may comprise a family B ligase. The ligase may be selected from the group consisting of T4 DNA ligase, T4 RNA ligase, Chlorella virus DNA ligase, Paramecium bursaria Chlorella virus 1 DNA ligase 1 (PBCV-1), T4 RNA ligase 1 (T4Rnl1), T4 RNA ligase 2 (T4Rnl2), DraRN1 ligase, KOD ligase, or Acanthocystic turfacea chlorella virus 1 (ATCV-1) ligase. The ligase may comprise a single stranded DNA ligase, or an Archaeal RNA ligase.

The instructions for using any of the methods are generally recorded on a suitable recording medium (e.g., printed on a substrate such as paper or plastic), or available in a digital format. As such, the instructions may be present in the kits as a package insert, in the labeling of the container of the kit or components thereof (i.e., associated with the packaging or subpackaging). In some cases, the instructions may be present as an electronic storage data file present on a suitable computer readable storage medium. In other cases, the actual instructions may not be present in the kit but means for obtaining the instructions from a remote source, e.g., via the internet, may be provided. For example, a kit that includes a web address where the instructions may be viewed and/or from which the instructions may be downloaded. As with the instructions, this means for obtaining the instructions is recorded on a suitable substrate.

Kits according to this aspect of the disclosure comprise a carrier means, such as a box, carton, tube or the like, having in close confinement therein one or more container means, such as vials, tubes, ampoules, bottles and the like. As used herein, a first container can contain one or more of the engineered family B polymerases or derivatives thereof of the present disclosure having reverse transcriptase activity. When one or more engineered family B polymerases having reverse transcriptase activity are used, the one or more engineered family B polymerases may be in a single container as mixtures of two or more engineered family B polymerases or derivatives thereof, or in separate containers. The kits of the disclosure can also comprise (in the same or separate containers) one or more DNA polymerases, a suitable buffer, one or more nucleotides and/or one or more primers.

The kits of the disclosure can also comprise one or more hosts or cells including those that are competent to take up nucleic acids (e.g., DNA molecules including vectors). Preferred hosts may include chemically competent or electrocompetent bacteria such as E. coli (including DH5, DH5α, DH10B, HB101, Top 10, and other K-12 strains as well as E. coli B and E. coli W strains).

In a specific aspect of the disclosure, the kits of the disclosure (e.g., reverse transcription and amplification kits) can include one or more components (in mixtures or separately) including one or more engineered family B polymerases or derivative thereof having reverse transcriptase activity of the disclosure, one or more nucleotides (one or more of which may be labeled, e.g., fluorescently labeled) used for synthesis of a nucleic acid molecule, and/or one or more primers (e.g., oligo(dT) for reverse transcription, randomers for extension reactions, etc.). Such kits can further comprise one or more DNA polymerases. Such kits can further comprise one or more ligases described herein.

### V. Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. “A and/or B” is used herein to include all of the following alternatives: “A”, “B”, “A or B”, and “A and B”. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “About” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed but are used merely as labels to distinguish one claim element having a certain name from another element having a same name (but for use of the ordinal term) to distinguish the claim elements. Similarly, the use of these terms in the specification does not by itself connote any required priority, precedence, or order.

By “analyte” is intended a biological molecule. Analytes include but are not limited to a DNA analyte, an RNA analyte, an oligonucleotide, a reporter molecule, a reporter molecule configured to directly couple to a protein, a reporter molecule configured to indirectly couple to a protein, a reporter molecule configured to directly couple to a metabolite, and a reporter molecule configured to indirectly couple to a metabolite.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcoded molecule with a nucleic acid sequence (e.g., nucleic acid sequence complementary to a nucleic acid primer sequence encompassed by the nucleic acid barcoded molecule). The nucleic acid sequence may be a targeted sequence or a non-targeted sequence. The nucleic acid barcoded molecule may be coupled to or attached to the nucleic acid molecule comprising the nucleic acid sequence. For example, a nucleic acid barcoded molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcoded molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcoded molecule to generate the barcoded nucleic acid molecule, or the nucleic acid molecule comprising the nucleic acid sequence may be attached to the nucleic acid barcoded molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “partitioning” as used herein is intended to encompass parting, dividing, depositing, separating, or compartmentalizing into one or more partitions. Systems and methods for partitioning of one or more particles (such as, but not limited to, biological particles, macromolecular constituents of biological particles, beads, reagents, etc.) into discrete compartments or partitions (referred to interchangeably here as partitions), where each partition maintains separation of its own content from the contents of other partitions are known in the art. See for example US 2020/0032335, herein incorporated by reference in its entirety. The partition can be a droplet in an emulsion. A partition may comprise one or more other partitions.

A “plurality” can mean at least about 2, at least about 3, at least about 4, at least about 5, at least about 6, at least about 7, at least about 8, at least about 9, at least about 10, at least about 25, at least about 50, at least about 75, at least about 100, at least about 150, at least about 200, at least about 300, at least about 400, at least about 500, at least about 1,000, at least about 1500, at least about 2000, at least about 2500, at least about 5,000, at least about 10,000, at least about 1,000,000, at least about 5,000,000, at least about 10,000,000 n, at least about 100,000,000, or at least about 1,000,000,000.

A “plurality of nucleic acid barcoded molecules” may comprise at least about 500 nucleic acid barcoded molecules, at least about 1,000 nucleic acid barcoded molecules, at least about 5,000 nucleic acid barcoded molecules, at least about 10,000 nucleic acid barcoded molecules, at least about 50,000 nucleic acid barcoded molecules, at least about 100,000 nucleic acid barcoded molecules, at least about 500,000 nucleic acid barcoded molecules, at least about 1,000,000 barcoded molecules, at least about 5,000,000 nucleic acid barcoded molecules, at least about 10,000,000 nucleic acid barcoded molecules, at least about 100,000,000 nucleic acid barcoded molecules, at least about 1,000,000,000 nucleic acid barcoded molecules. In some cases, a plurality of nucleic acid barcoded molecules comprise a partition-specific barcode sequence.

Each of the plurality of nucleic acid barcoded molecules may include an identifier sequence separate from the partition-specific barcode sequence, where the identifier sequence is different for each nucleic acid partition-specific barcoded molecule of the plurality of nucleic acid partition specific barcoded molecules. In some cases, such an identifier sequence is a unique molecular identifier (UMI) as described elsewhere herein. As described elsewhere herein, UMI sequences can uniquely identify a particular nucleic acid molecule that is barcoded, which may be identifying particular nucleic acid molecules that are analyzed, counting particular nucleic acid molecules that are analyzed, etc. Furthermore, in some cases, each of the plurality of nucleic acid barcoded molecules can comprise the partition specific barcode sequence and the bead can be from plurality of beads, such as a population of barcoded beads. Each of the partition specific barcode sequences can be different from partition specific barcode sequences of nucleic acid barcoded molecules of other beads of the plurality of beads. Where this is the case, a population of barcoded beads, with each bead comprising a different partition specific barcode sequence can be analyzed.

As used herein, the terms “unique molecular identifier”, “unique molecular identifying sequence”, “UMI” and “UMI sequence” are used synonymously. Individual barcoded molecules may comprise a common barcode sequence such as a partition specific sequence or a spatial array where every capture probe has a unique barcode sequence.

By “binding sequence” is intended a nucleic acid sequence capable of binding to an analyte.

A nucleic acid barcoded molecule of a plurality of nucleic acid molecules may be used to generate a “barcoded nucleic acid molecule.” In some cases, a barcoded molecule comprises a different reporter barcode sequence that identifies a second analyte. A different reporter barcode sequence or an analyte-specific barcode sequence may identify a protein, a lipid, a metabolite or other second analyte.

As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

As used herein, a “first probe” can refer to a probe that hybridizes to all or a portion of an analyte and can be ligated to one or more additional probes (e.g., a second probe or a spanning probe). In some embodiments, “first probe” can be used interchangeably with “first probe oligonucleotide.”

In some embodiments, the first probe includes ribonucleotides, deoxyribonucleotides, and/or synthetic nucleotides that are capable of participating in Watson-Crick type or analogous base pair interactions. In some embodiments, the first probe includes deoxyribonucleotides. In some embodiments, the first probe includes deoxyribonucleotides and ribonucleotides. In some embodiments, the first probe includes a deoxyribonucleic acid that hybridizes to an analyte and includes a portion of the oligonucleotide that is not a deoxyribonucleic acid. For example, in some embodiments, the portion of the first oligonucleotide that is not a deoxyribonucleic acid is a ribonucleic acid or any other non-deoxyribonucleic acid nucleic acid as described herein. In some embodiments where the first probe includes deoxyribonucleotides, hybridization of the first probe to the mRNA molecule results in a DNA:RNA hybrid. In some embodiments, the first probe includes only deoxyribonucleotides and upon hybridization of the first probe to the mRNA molecule results in a DNA:RNA hybrid.

In some embodiments, the method includes a first probe that includes one or more sequences that are substantially complementary to one or more sequences of an analyte. In some embodiments, a first probe includes a sequence that is substantially complementary to a first target sequence in the analyte. In some embodiments, the sequence of the first probe that is substantially complementary to the first target sequence in the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the analyte.

In some embodiments, a first probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a first probe includes a functional sequence. In some embodiments, a functional sequence includes a primer sequence. In some embodiments, a first probe includes at least two ribonucleic acid bases at the 3′ end. In such cases, a second probe oligonucleotide comprises a phosphorylated nucleotide at the 5′ end. In some embodiments, a first probe includes at least three, at least four, at least five, at least six, at least seven, at least eight, at least nine, or at least ten ribonucleic acid bases at the 3′ end.

As used herein, a “second probe” can refer to a probe that hybridizes to all or a portion of an analyte and can be ligated to one or more additional probes (e.g., a first probe or a spanning probe). In some embodiments, “second probe” can be used interchangeably with “second probe oligonucleotide.” One of skill in the art will appreciate that the order of the probes is arbitrary, and thus the contents of the first probe and/or second probe as disclosed herein are interchangeable.

In some embodiments, the second probe includes ribonucleotides, deoxyribonucleotides, and/or synthetic nucleotides that are capable of participating in Watson-Crick type or analogous base pair interactions. In some embodiments, the second probe includes deoxyribonucleotides. In some embodiments, the second probe includes deoxyribonucleotides and ribonucleotides. In some embodiments, the second probe includes a deoxyribonucleic acid that hybridizes to an analyte and includes a portion of the oligonucleotide that is not a deoxyribonucleic acid. For example, in some embodiments, the portion of the second probe that is not a deoxyribonucleic acid is a ribonucleic acid or any other non-deoxyribonucleic acid nucleic acid as described herein. In some embodiments where the second probe includes deoxyribonucleotides, hybridization of the second probe to the mRNA molecule results in a DNA:RNA hybrid. In some embodiments, the second probe includes only deoxyribonucleotides and upon hybridization of the first probe to the mRNA molecule results in a DNA:RNA hybrid.

In some embodiments, the method includes a second probe that includes one or more sequences that are substantially complementary to one or more sequences of an analyte. In some embodiments, a second probe includes a sequence that is substantially complementary to a second target sequence in the analyte. In some embodiments, the sequence of the second probe that is substantially complementary to the second target sequence in the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence in the analyte.

In some embodiments, a second probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

As used herein, a “capture probe capture domain” is a sequence, domain, or moiety that can bind specifically to a capture domain of a capture probe. In some embodiments, “capture domain capture domain” can be used interchangeably with “capture probe binding domain.” In some embodiments, a second probe includes a sequence from 5′ to 3′: a sequence that is substantially complementary to a sequence in the analyte and a capture probe capture domain.

In some embodiments, a capture probe capture domain includes a poly(A) sequence. In some embodiments, the capture probe capture domain includes a poly-uridine sequence, a poly-thymidine sequence, or both. In some embodiments, the capture probe capture domain includes a random sequence (e.g., a random hexamer or octamer). In some embodiments, the capture probe capture domain is complementary to a capture domain in a capture probe that detects a particular target(s) of interest. In some embodiments, a capture probe capture domain blocking moiety that interacts with the capture probe capture domain is provided. In some embodiments, a capture probe capture domain blocking moiety includes a sequence that is complementary or substantially complementary to a capture probe capture domain. In some embodiments, a capture probe capture domain blocking moiety prevents the capture probe capture domain from binding the capture probe when present.

In some embodiments, a capture probe capture domain blocking moiety is removed prior to binding the capture probe capture domain (e.g., present in a ligated probe) to a capture probe. In some embodiments, a capture probe capture domain blocking moiety includes a poly-uridine sequence, a poly-thymidine sequence, or both. In some embodiments, the capture probe capture domain sequence includes ribonucleotides, deoxyribonucleotides, and/or synthetic nucleotides that are capable of participating in Watson-Crick type or analogous base pair interactions. In some embodiments, the capture probe binding domain sequence includes at least 10, 11, 12, 13, 14, 15, 16, 17, 18, 19, or 20 nucleotides. In some embodiments, the capture probe binding domain sequence includes at least 25, 30, or 35 nucleotides.

In some embodiments, a second probe includes a phosphorylated nucleotide at the 5′ end. The phosphorylated nucleotide at the 5′ end can be used in a ligation reaction to ligate the second probe to the first probe.

As used herein, the term “operably linked” or “conjugated” or “fusion” means that, in relation to the recombinant thermostable polymerase enzyme sequence there are one or more sequences at the N or C terminus that, when transcribed and translated, create additional polypeptides in association with the enzyme amino acid sequence, thereby created a conjugation or fusion of one or more polypeptides from one expression vector.

As used herein, the term “reverse transcriptase activity,” “reverse transcription activity,” or “reverse transcription” indicates the capability of an enzyme to synthesize a DNA strand (that is, complementary DNA or cDNA) using RNA as a template.

As used herein, the term “mutation” or “mutant” or “variant” indicates a change or changes introduced in a wildtype DNA sequence or a wildtype amino acid sequence. Examples of mutations or variants include, but are not limited to, substitutions, insertions, deletions, and point mutations. Mutations can be made either at the nucleic acid level or at the amino acid level.

As used herein, the term “thermoreactivity” or “thermoreactive” refers to the ability of a reverse transcriptase to exhibit enzyme activity at elevated temperatures.

As used herein, “thermostability” or “thermostable” refers to the ability of a reverse transcriptase to withstand exposure to elevated temperatures, but not necessarily show activity at such elevated temperatures. In some embodiments, thermostable reverse transcriptase (e.g., the engineered family B polymerase) or polymerase refers to any enzyme that catalyzes polynucleotide synthesis by addition of nucleotide units to a nucleotide chain using DNA or RNA as a template and has an optimal activity at a temperature above 53° C.

As used herein, the term “processivity” refers to the ability of a reverse transcriptase to continuously extend a primer without disassociating from the nucleic acid template. The length of a template a reverse transcriptase or polymerase is capable of replicating can also be used to describe the processivity of that reverse transcriptase or polymerase. In some embodiments, “Processivity” refers to the ability of a polymerase to remain bound to the template or substrate and perform DNA synthesis. Processivity is measured by the number of catalytic events that take place per binding event.

As used herein, the term “inhibitor resistance” refers to the ability of a reverse transcriptase to perform reverse transcription in the presence of a compound, chemical, protein, buffer, etc. that is typically inhibitory to the reverse transcriptase (prevents or inhibits reverse transcriptase activity).

As used herein, the term “fidelity” refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

As used herein, the term “identical” in the context of two nucleic acids or polypeptide sequences refers to the residues in the two sequences that are the same when aligned for maximum correspondence, as measured using a sequence comparison algorithms. Sequence comparison algorithms are known to those skill in the art. See e.g., ebi.ac.uk/Tools/msa/clustalo/.

As used herein, the term “efficiency” in the context of a nucleic acid modifying enzyme of this disclosure refers to the ability of the enzyme to perform its catalytic function under specific reaction conditions. Typically, “efficiency” as defined herein is indicated by the amount of product generated under given reaction conditions.

As used herein, the term “enhances” in the context of an enzyme refers to improving the activity of the enzyme, i.e., increasing the amount of product per unit enzyme per unit time.

As used herein, the term “strand-displacing polymerase”, refers to a polymerase that is able to displace one or more nucleotides, such as at least 10 or 100 or more nucleotides that are downstream from the enzyme. Strand displacing polymerases can be differentiated from non-strand displacing polymerase. In some embodiments, the strand displacing polymerase is stable and active at a temperature of at least 50° C. or at least 55° C. (including the strand displacing activity). Taq polymerase is a nick translating polymerase and, as such, is not a strand displacing polymerase.

### VI. Sequences

Wild-Type Pyrococcus furiosus (pfu) DNA polymerase; NCBI
Reference Sequence: WP_011011325.1
SEQ ID NO: 1
MILDVDYITEEGKPVIRLFKKENGKFKIEHDRTFRPYIYALLRDDSKIEEVKKITGERH
GKIVRIVDVEKVEKKFLGKPITVWKLYLEHPQDVPTIREKVREHPAVVDIFEYDIPFA
KRYLIDKGLIPMEGEEELKILAFDIETLYHEGEEFGKGPIIMISYADENEAKVITWKNID
LPYVEVVSSEREMIKRFLRIIREKDPDIIVTYNGDSFDFPYLAKRAEKLGIKLTIGRDGS
EPKMQRIGDMTAVEVKGRIHFDLYHVITRTINLPTYTLEAVYEAIFGKPKEKVYADEI
AKAWESGENLERVAKYSMEDAKATYELGKEFLPMEIQLSRLVGQPLWDVSRSSTGN
LVEWFLLRKAYERNEVAPNKPSEEEYQRRLRESYTGGFVKEPEKGLWENIVYLDFR
ALYPSIIITHNVSPDTLNLEGCKNYDIAPQVGHKFCKDIPGFIPSLLGHLLEERQKIKTK
MKETQDPIEKILLDYRQKAIKLLANSFYGYYGYAKARWYCKECAESVTAWGRKYIE
LVWKELEEKFGFKVLYIDTDGLYATIPGGESEEIKKKALEFVKYINSKLPGLLELEYE
GFYKRGFFVTKKRYAVIDEEGKVITRGLEIVRRDWSEIAKETQARVLETILKHGDVEE
AVRIVKEVIQKLANYEIPPEKLAIYEQITRPLHEYKAIGPHVAVAKKLAAKGVKIKPG
MVIGYIVLRGDGPISNRAILAEEYDPKKHKYDAEYYIENQVLPAVLRILEGFGYRKED
LRYQKTRQVGLTSWLNIKKS
Pfu-RTX
SEQ ID NO: 2
MILDVDYITEEGKPVIRLFKKENGKFKIEHDRTFRPYLYALLRDDSKIEEVKKITGERH
GKIVRIVDVEKVEKKFLGKPITVWKLYLEHPQDVPTIMEKVREHPAVVDIFEYDIPFAI
RYLIDKGLIPMEGEEELKLLAFDIETLYHEGEEFGKGPIIMISYADENEAKVITWKNID
LPYVEVVSSEREMIKRFLRIIREKDPDIIVTYNGDSFDFPYLAKRAEKLGIKLTIGRDGS
EPKMQRIGDMTAVEVKGRIHFDLYHVITRTINLPTYTLEAVYEAIFGKPKEKVYADEI
AKAWESGENLERVAKYSMEDAKATYELGKEFLPMEIQLSRLVGQPLWDVSRSSTGN
LVEWFLLRKAYERNEVAPNKPSEEEYQRRLHESHTGGFIKEPEKGLWENIVYLDFRA
LYPSIIITHNVSPDTLNLEGCKNYDIAPQVGHKFCKDIPGFIPSLLGHLLEERQKIKTRM
KETQDPIEKILLDYRQKAIKLLANSLYGYYGYAKARWYCKECAESVIAWGRKYLEL
VWKELEEKFGFKVLYIDTDGLYATIPGGESEEIKKKALEFVKYINSKLPGLLELEYEG
FYKRGLFVTKKRYAVIDEEGKVITRGLEIVRRDWSEIAKETQARVLETILKHGDVEEA
VRIVKEVIQKLANYEIPPEKLAIYKQITRPLHEYKAIGPHVAVAKKLAAKGVKIKPGM
VIGYIVLRGDGPIVNRAILAEEYDPKKHKYDAEYYIEKQVLPAVLRILEGFGYRKEDL
RYQKTRQVGLTSRLNIKKS
Pfu-RTXxo-1
SEQ ID NO: 3
MILDVDYITEEGKPVIRLFKKENGKFKIEHDRTFRPYLYALLRDDSKIEEVKKITGERH
GKIVRIVDVEKVEKKFLGKPITVWKLYLEHPQDVPTIMEKVREHPAVVDIFEYDIPFAI
RYLIDKGLIPMEGEEELKLLAFAIATLYHEGEEFGKGPIIMISYADENEAKVITWKNID
LPYVEVVSSEREMIKRFLRIIREKDPDIIVTYNGDSFDFPYLAKRAEKLGIKLTIGRDGS
EPKMQRIGDMTAVEVKGRIHFDLYHVITRTINLPTYTLEAVYEAIFGKPKEKVYADEI
AKAWESGENLERVAKYSMEDAKATYELGKEFLPMEIQLSRLVGQPLWDVSRSSTGN
LVEWFLLRKAYERNEVAPNKPSEEEYQRRLHESHTGGFIKEPEKGLWENIVYLDFRA
LYPSIIITHNVSPDTLNLEGCKNYDIAPQVGHKFCKDIPGFIPSLLGHLLEERQKIKTRM
KETQDPIEKILLDYRQKAIKLLANSLYGYYGYAKARWYCKECAESVIAWGRKYLEL
VWKELEEKFGFKVLYIDTDGLYATIPGGESEEIKKKALEFVKYINSKLPGLLELEYEG
FYKRGLFVTKKRYAVIDEEGKVITRGLEIVRRDWSEIAKETQARVLETILKHGDVEEA
VRIVKEVIQKLANYEIPPEKLAIYKQITRPLHEYKAIGPHVAVAKKLAAKGVKIKPGM
VIGYIVLRGDGPIVNRAILAEEYDPKKHKYDAEYYIEKQVLPAVLRILEGFGYRKEDL
RYQKTRQVGLTSRLNIKKS
Pfu-RTXxo-2
SEQ ID NO: 4
MVLDVDYITEEGKPVIRLFKKENGKFKIEHDRTFRPYLYALLRDDSKIEEVKKITGER
HGKIVRIVDVEKVEKKFLGKPITVWKLYLEHPQDVPTIMEKVREHPAVVDIFEYDIPF
AIRYLIDKGLIPMEGEEELKLLAFAIATLYHEGEEFGKGPIIMISYADENEAKVITWKNI
DLPYVEVVSSEREMIKRFLRIIREKDPDIIVTYNGDSFDFPYLAKRAEKLGIKLTIGRDG
SEPKMQRIGDMTAVEVKGRIHFDLYHVITRTINLPTYTLEAVYEAIFGKPKEKVYADE
IAKAWESGENLERVAKYSMEDAKATYELGKEFLPMEIQLSRLVGQPLWDVSRSSTG
NLVEWFLLRKAYERNEVAPNKPSEEEYQRRLHESHTGGFIKEPEKGLWENIVYLDFR
ALYPSIIITHNVSPDTLNLEGCKNYDIAPQVGHKFCKDIPGFIPSLLGHLLEERQKIKTR
MKETQDPIEKILLDYRQKAIKLLANSLYGYYGYAKARWYCKECAESVIAWGRKYLE
LVWKELEEKFGFKVLYIDTDGLYATIPGGESEEIKKKALEFVKYINSKLPGLLELEYE
GFYKRGLFVTKKRYAVIDEEGKVITRGLEIVRRDWSEIAKETQARVLETILKHGDVEE
AVRIVKEVIQKLANYEIPPEKLAIYKQITRPLHEYKAIGPHVAVAKKLAAKGVKIKPG
MVIGYIVLRGDGPIVNRAILAEEYDPKKHKYDAEYYIEKQVLPAVLRILEGFGYRKED
LRYQKTRQVGLTSRLNIKKS
Pfu-RTXxo-3
SEQ ID NO: 5
MVLDVDYITEEGKPVIRLFKKENGKFKIEHDRTFRPYLYALLRDDSKIEEVKKITGER
HGKIVRIVDVEKVEKKFLGKPITVWKLYLEHPQDQPTIMEKVREHPAVVDIFEYDIPF
AIRYLIDKGLIPMEGEEELKLLAFAIATLYHEGEEFGKGPIIMISYADENEAKVITWKNI
DLPYVEVVSSEREMIKRFLRIIREKDPDIIVTYNGDSFDFPYLAKRAEKLGIKLTIGRDG
SEPKMQRIGDMTAVEVKGRIHFDLYHVITRTINLPTYTLEAVYEAIFGKPKEKVYADE
IAKAWESGENLERVAKYSMEDAKATYELGKEFLPMEIQLSRLVGQPLWDVSRSSTG
NLVEWFLLRKAYERNEVAPNKPSEEEYQRRLHESHTGGFIKEPEKGLWENIVYLDFR
ALYPSIIITHNVSPDTLNLEGCKNYDIAPQVGHKFCKDIPGFIPSLLGHLLEERQKIKTR
MKETQDPIEKILLDYRQKLIKLLANSLYGYYGYAKARWYCKECAESVIAWGRKYLE
LVWKELEEKFGFKVLYIDTDGLYATIPGGESEEIKKKALEFVKYINSKLPGLLELEYE
GFYKRGLFVTKKRYAVIDEEGKVITRGLEIVRRDWSEIAKETQARVLETILKHGDVEE
AVRIVKEVIQKLANYEIPPEKLAIYKQITRPLHEYKAIGPHVAVAKKLAAKGVKIKPG
MVIGYIVLRGDGPIVNRAILAEEYDPKKHKYDAEYYIEKQVLPAVLRILEGFGYRKED
LRYQKTRQVGLTSRLNIKKS
Wild-Type Thermococus kodakarensis (KOD1) polymerase;
KodPol; NCBI Reference Sequence: 1WNS_A
SEQ ID NO: 6
MILDTDYITEDGKPVIRIFKKENGEFKIEYDRTFEPYFYALLKDDSAIEEVKKITAERH
GTVVTVKRVEKVQKKFLGRPVEVWKLYFTHPQDVPAIRDKIREHPAVIDIYEYDIPFA
KRYLIDKGLVPMEGDEELKMLAFDIETLYHEGEEFAEGPILMISYADEEGARVITWK
NVDLPYVDVVSTEREMIKRFLRVVKEKDPDVLITYNGDNFDFAYLKKRCEKLGINFA
LGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVS
RSSTGNLVEWFLLRKAYERNELAPNKPDEKELARRRQSYEGGYVKEPERGLWENIV
YLDFRSLYPSIHITHNVSPDTLNREGCKEYDVAPQVGHRFCKDFPGFIPSLLGDLLEER
QKIKKKMKATIDPIERKLLDYRQRAIKILANSYYGYYGYARARWYCKECAESVTAW
GREYITMTIKEIEEKYGFKVIYSDTDGFFATIPGADAETVKKKAMEFLKYINAKLPGA
LELEYEGFYKRGFFVTKKKYAVIDEEGKITTRGLEIVRRDWSEIAKETQARVLEALLK
DGDVEKAVRIVKEVTEKLSKYEVPPEKLVIHEQITRDLKDYKATGPHVAVAKRLAA
RGVKIRPGTVISYIVLKGSGRIGDRAIPFDEFDPTKHKYDAEYYIENQVLPAVERILRA
FGYRKEDLRYQKTRQVGLSAWLKPKGT
KOD-RTX
SEQ ID NO: 7
MILDTDYITEDGKPVIRIFKKENGEFKIEYDRTFEPYLYALLKDDSAIEEVKKITAERH
GTVVTVKRVEKVQKKFLGRPVEVWKLYFTHPQDVPAIMDKIREHPAVIDIYEYDIPF
AIRYLIDKGLVPMEGDEELKLLAFDIETLYHEGEEFAEGPILMISYADEEGARVITWK
NVDLPYVDVVSTEREMIKRFLRVVKEKDPDVLITYNGDNFDFAYLKKRCEKLGINFA
LGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVS
RSSTGNLVEWFLLRKAYERNELAPNKPDEKELARRHQSHEGGYIKEPERGLWENIVY
LDFRSLYPSIIITHNVSPDTLNREGCKEYDVAPQVGHRFCKDFPGFIPSLLGDLLEERQ
KIKKRMKATIDPIERKLLDYRQRAIKILANSLYGYYGYARARWYCKECAESVIAWGR
EYLTMTIKEIEEKYGFKVIYSDTDGFFATIPGADAETVKKKAMEFLKYINAKLPGALE
LEYEGFYKRGLFVTKKKYAVIDEEGKITTRGLEIVRRDWSEIAKETQARVLEALLKD
GDVEKAVRIVKEVTEKLSKYEVPPEKLVIHKQITRDLKDYKATGPHVAVAKRLAAR
GVKIRPGTVISYIVLKGSGRIVDRAIPFDEFDPTKHKYDAEYYIEKQVLPAVERILRAF
GYRKEDLRYQKTRQVGLSARLKPKGT
Wild-Type KodPol (NCBI PDB: 1WN7_A)
SEQ ID NO: 8
MILDTDYITEDGKPVIRIFKKENGEFKIEYDRTFEPYFYALLKDDSAIEEVKKITAERH
GTVVTVKRVEKVQKKFLGRPVEVWKLYFTHPQDVPAIRDKIREHPAVIDIYEYDIPFA
KRYLIDKGLVPMEGDEELKMLAFDIETLYEEGEEFAEGPILMISYADEEGARVITWKN
VDLPYVDVVSTEREMIKRFLRVVKEKDPDVLITYNGDNFDFAYLKKRCEKLGINFAL
GRDGSEPKIQRMGDRFAVEVKGRIHFDLYPVIRRTINLPTYTLEAVYEAVFGQPKEKV
YAEEITTAWETGENLERVARYSMEDAKVTYELGKEFLPMEAQLSRLIGQSLWDVSR
SSTGNLVEWFLLRKAYERNELAPNKPDEKELARRRQSYEGGYVKEPERGLWENIVY
LDFRSLYPSIIITHNVSPDTLNREGCKEYDVAPQVGHRFCKDFPGFIPSLLGDLLEERQ
KIKKKMKATIDPIERKLLDYRQRAIKILANSYYGYYGYARARWYCKECAESVTAWG
REYITMTIKEIEEKYGFKVIYSDTDGFFATIPGADAETVKKKAMEFLKYINAKLPGAL
ELEYEGFYERGFFVTKKKYAVIDEEGKITTRGLEIVRRDWSEIAKETQARVLEALLKD
GDVEKAVRIVKEVTEKLSKYEVPPEKLVIHEQITRDLKDYKATGPHVAVAKRLAAR
GVKIRPGTVISYIVLKGSGRIGDRAIPFDEFDPTKHKYDAEYYIENQVLPAVERILRAF
GYRKEDLRYQKTRQVGLSAWLKPKGT
KOD-RTX
SEQ ID NO: 9
MILDTDYITEDGKPVIRIFKKENGEFKIEYDRTFEPYLYALLKDDSAIEEVKKITAERH
GTVVTVKRVEKVQKKFLGRPVEVWKLYFTHPQDVPAIMDKIREHPAVIDIYEYDIPF
AIRYLIDKGLVPMEGDEELKLLAFDIETLYEEGEEFAEGPILMISYADEEGARVITWK
NVDLPYVDVVSTEREMIKRFLRVVKEKDPDVLITYNGDNFDFAYLKKRCEKLGINFA
LGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVS
RSSTGNLVEWFLLRKAYERNELAPNKPDEKELARRHQSHEGGYIKEPERGLWENIVY
LDFRSLYPSIIITHNVSPDTLNREGCKEYDVAPQVGHRFCKDFPGFIPSLLGDLLEERQ
KIKKRMKATIDPIERKLLDYRQRAIKILANSLYGYYGYARARWYCKECAESVIAWGR
EYLTMTIKEIEEKYGFKVIYSDTDGFFATIPGADAETVKKKAMEFLKYINAKLPGALE
LEYEGFYERGLFVTKKKYAVIDEEGKITTRGLEIVRRDWSEIAKETQARVLEALLKD
GDVEKAVRIVKEVTEKLSKYEVPPEKLVIHKQITRDLKDYKATGPHVAVAKRLAAR
GVKIRPGTVISYIVLKGSGRIVDRAIPFDEFDPTKHKYDAEYYIEKQVLPAVERILRAF
GYRKEDLRYQKTRQVGLSARLKPKGT
Wild-type Thermococcus gorgonarius (Tgo); TgoPol; NCBI
Reference Sequence: WP_088885078.1
SEQ ID NO: 10
MILDTDYITEDGKPVIRIFKKENGEFKIDYDRNFEPYIYALLKDDSAIEDVKKITAERH
GTTVRVVRAEKVKKKFLGRPIEVWKLYFTHPQDVPAIRDKIKEHPAVVDIYEYDIPFA
KRYLIDKGLIPMEGDEELKMLAFDIETLYHEGEEFAEGPILMISYADEEGARVITWKN
IDLPYVDVVSTEKEMIKRFLKVVKEKDPDVLITYNGDNFDFAYLKKRSEKLGVKFIL
GREGSEPKIQRMGDRFAVEVKGRIHFDLYPVIRRTINLPTYTLEAVYEAIFGQPKEKV
YAEEIAQAWETGEGLERVARYSMEDAKVTYELGKEFFPMEAQLSRLVGQSLWDVS
RSSTGNLVEWFLLRKAYERNELAPNKPDERELARRRESYAGGYVKEPERGLWENIV
YLDFRSLYPSIHITHNVSPDTLNREGCEEYDVAPQVGHKFCKDFPGFIPSLLGDLLEER
QKVKKKMKATIDPIEKKLLDYRQRAIKILANSFYGYYGYAKARWYCKECAESVTA
WGRQYIETTIREIEEKFGFKVLYADTDGFFATIPGADAETVKKKAKEFLDYINAKLPG
LLELEYEGFYKRGFFVTKKKYAVIDEEDKITTRGLEIVRRDWSEIAKETQARVLEAIL
KHGDVEEAVRIVKEVTEKLSKYEVPPEKLVIYEQITRDLKDYKATGPHVAVAKRLAA
RGIKIRPGTVISYIVLKGSGRIGDRAIPFDEFDPAKHKYDAEYYIENQVLPAVERILRAF
GYRKEDLRYQKTRQVGLGAWLKPKT
TgoRTxo (No proofreading)
SEQ ID NO: 11
MVLDTDYITEDGKPVIRIFKKENGEFKIDYDRNFEPYLYALLKDDSAIEDVKKITAER
HGTTVRVVRAEKVKKKFLGRPIEVWKLYFTHPQDVPAIMDKIKEHPAVVDIYEYDIP
FAIRYLIDKGLIPMEGDEELKLLAFAIATLYHEGEEFAEGPILMISYADEEGARVITWK
NIDLPYVDVVSTEKEMIKRFLKVVKEKDPDVLITYNGDNFDFAYLKKRSEKLGVKFI
LGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDV
SRSSTGNLVEWFLLRKAYERNELAPNKPDERELARRHESHAGGYIKEPERGLWENIV
YLDFRSLYPSIHITHNVSPDTLNREGCEEYDVAPQVGHKFCKDFPGFIPSLLGDLLEER
QKVKKRMKATIDPIEKKLLDYRQRAIKILANSLYGYYGYAKARWYCKECAESVIAW
GRQYLETTIREIEEKFGFKVLYADTDGFFATIPGADAETVKKKAKEFLDYINAKLPGL
LELEYEGFYKRGLFVTKKKYAVIDEEDKITTRGLEIVRRDWSEIAKETQARVLEAILK
HGDVEEAVRIVKEVTEKLSKYEVPPEKLVIYKQITRDLKDYKATGPHVAVAKRLAA
RGIKIRPGTVISYIVLKGSGRIVDRAIPFDEFDPAKHKYDAEYYIEKQVLPAVERILRAF
GYRKEDLRYQKTRQVGLGARLKPKTLEHHHHHH
TgoRT (with proofreading)
SEQ ID NO: 12
MVLDTDYITEDGKPVIRIFKKENGEFKIDYDRNFEPYLYALLKDDSAIEDVKKITAER
HGTTVRVVRAEKVKKKFLGRPIEVWKLYFTHPQDVPAIMDKIKEHPAVVDIYEYDIP
FAIRYLIDKGLIPMEGDEELKLLDFEIATLYHEGEEFAEGPILMISYADEEGARVITWK
NIDLPYVDVVSTEKEMIKRFLKVVKEKDPDVLITYNGDNFDFAYLKKRSEKLGVKFI
LGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDV
SRSSTGNLVEWFLLRKAYERNELAPNKPDERELARRHESHAGGYIKEPERGLWENIV
YLDFRSLYPSIHITHNVSPDTLNREGCEEYDVAPQVGHKFCKDFPGFIPSLLGDLLEER
QKVKKRMKATIDPIEKKLLDYRQRAIKILANSLYGYYGYAKARWYCKECAESVIAW
GRQYLETTIREIEEKFGFKVLYADTDGFFATIPGADAETVKKKAKEFLDYINAKLPGL
LELEYEGFYKRGLFVTKKKYAVIDEEDKITTRGLEIVRRDWSEIAKETQARVLEAILK
HGDVEEAVRIVKEVTEKLSKYEVPPEKLVIYKQITRDLKDYKATGPHVAVAKRLAA
RGIKIRPGTVISYIVLKGSGRIVDRAIPFDEFDPAKHKYDAEYYIEKQVLPAVERILRAF
GYRKEDLRYQKTRQVGLGARLKPKTLEHHHHHH
a histidine purification tag
SEQ ID NO: 13
HHHHHH
short peptide C-terminal tag
SEQ ID NO: 14
SEEDEEKEEDG
Tobacco etch virus protease (TEV) cleavage site
>SEQ ID NO: 15
ENLYFQ/G
Enterokinase (EntK) cleavage site
SEQ ID NO: 16
DDDDK/
Factor Xa (Xa) cleavage site
SEQ ID NO: 17
IEGR/
Thrombin (Thr) cleavage site
SEQ ID NO: 18
LVPR/GS
Genetically engineered derivative of human rhinovirus 3C
protease cleavage site
SEQ ID NO: 19
LEVLFQ/GP
VENT® polymerase; AAA72101.1 DNA dependent DNA
polymerase [Thermococcus litoralis]
SEQ ID NO: 20
MILDTDYITKDGKPIIRIFKKENGEFKIELDPHFQPYIYALLKDDSAIEEIKAIKGERHG
KTVRVLDAVKVRKKFLGREVEVWKLIFEHPQDVPAMRGKIREHPAVVDIYEYDIPFA
KRYLIDKGLIPMEGDEELKLLAFDIETFYHEGDEFGKGEIIMISYADEEEARVITWKNI
DLPYVDVVSNEREMIKRFVQVVKEKDPDVIITYNGDNFDLPYLIKRAEKLGVRLVLG
RDKEHPEPKIQRMGDSFAVEIKGRIHFDLFPVVRRTINLPTYTLEAVYEAVLGKTKSK
LGAEEIAAIWETEESMKKLAQYSMEDARATYELGKEFFPMEAELAKLIGQSVWDVS
RSSTGNLVEWYLLRVAYARNELAPNKPDEEEYKRRLRTTYLGGYVKEPEKGLWENI
IYLDFRSLYPSIIVTHNVSPDTLEKEGCKNYDVAPIVGYRFCKDFPGFIPSILGDLIAMR
QDIKKKMKSTIDPIEKKMLDYRQRAIKLLANSYYGYMGYPKARWYSKECAESVTA
WGRHYIEMTIREIEEKFGFKVLYADTDGFYATIPGEKPELIKKKAKEFLNYINSKLPGL
LELEYEGFYLRGFFVTKKRYAVIDEEGRITTRGLEVVRRDWSEIAKETQAKVLEAILK
EGSVEKAVEVVRDVVEKIAKYRVPLEKLVIHEQITRDLKDYKAIGPHVAIAKRLAAR
GIKVKPGTIISYIVLKGSGKISDRVILLTEYDPRKHKYDPDYYIENQVLPAVLRILEAFG
YRKEDLRYQSSKQTGLDAWLKR
Deep Vent® polymerase; AAA67131.1 DNA polymerase
[Pyrococcus sp.]
SEQ ID NO: 21
MILDADYITEDGKPIIRIFKKENGEFKVEYDRNFRPYIYALLKDDSQIDEVRKITAERH
GKIVRIIDAEKVRKKFLGRPIEVWRLYFEHPQDVPAIRDKIREHSAVIDIFEYDIPFAKR
YLIDKGLIPMEGDEELKLLAFDIETLYHEGEEFAKGPIIMISYADEEEAKVITWKKIDL
PYVEVVSSEREMIKRFLKVIREKDPDVIITYNGDSFDLPYLVKRAEKLGIKLPLGRDGS
EPKMQRLGDMTAVEIKGRIHFDLYHVIRRTINLPTYTLEAVYEAIFGKPKEKVYAHEI
AEAWETGKGLERVAKYSMEDAKVTYELGREFFPMEAQLSRLVGQPLWDVSRSSTG
NLVEWYLLRKAYERNELAPNKPDEREYERRLRESYAGGYVKEPEKGLWEGLVSLDF
RSLYPSIIITHNVSPDTLNREGCREYDVAPEVGHKFCKDFPGFIPSLLKRLLDERQEIKR
KMKASKDPIEKKMLDYRQRAIKILANSYYGYYGYAKARWYCKECAESVTAWGREY
IEFVRKELEEKFGFKVLYIDTDGLYATIPGAKPEEIKKKALEFVDYINAKLPGLLELEY
EGFYVRGFFVTKKKYALIDEEGKIITRGLEIVRRDWSEIAKETQAKVLEAILKHGNVE
EAVKIVKEVTEKLSKYEIPPEKLVIYEQITRPLHEYKAIGPHVAVAKRLAARGVKVRP
GMVIGYIVLRGDGPISKRAILAEEFDLRKHKYDAEYYIENQVLPAVLRILEAFGYRKE
DLRWQKTKQTGLTAWLNIKKK
9º N. polymerase; Q56366.1; DNA polymerase [Thermococcus
sp. 9° N.-7]
SEQ ID NO: 22
MILDTDYITENGKPVIRVFKKENGEFKIEYDRTFEPYFYALLKDDSAIEDVKKVTAKR
HGTVVKVKRAEKVQKKFLGRPIEVWKLYFNHPQDVPAIRDRIRAHPAVVDIYEYDIP
FAKRYLIDKGLIPMEGDEELTMLAFDIETLYHEGEEFGTGPILMISYADGSEARVITW
KKIDLPYVDVVSTEKEMIKRFLRVVREKDPDVLITYNGDNFDFAYLKKRCEELGIKFT
LGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVS
RSSTGNLVEWFLLRKAYKRNELAPNKPDERELARRRGGYAGGYVKEPERGLWDNIV
YLDFRSLYPSIIITHNVSPDTLNREGCKEYDVAPEVGHKFCKDFPGFIPSLLGDLLEER
QKIKRKMKATVDPLEKKLLDYRQRAIKILANSFYGYYGYAKARWYCKECAESVTA
WGREYIEMVIRELEEKFGFKVLYADTDGLHATIPGADAETVKKKAKEFLKYINPKLP
GLLELEYEGFYVRGFFVTKKKYAVIDEEGKITTRGLEIVRRDWSEIAKETQARVLEAI
LKHGDVEEAVRIVKEVTEKLSKYEVPPEKLVIHEQITRDLRDYKATGPHVAVAKRLA
ARGVKIRPGTVISYIVLKGSGRIGDRAIPADEFDPTKHRYDAEYYIENQVLPAVERILK
AFGYRKEDLRYQKTKQVGLGAWLKVKGKK
42B (RTx_His_(MMLV variant))
SEQ ID NO: 23
ACTTGGCTGTCTGATTTCCCTCAGGCGTGGGCCGAAACGGGTGGCATGGGTCTGG
CAGTGCGTCAGGCACCGCTGATTATTCCGCTGAAAGCGACGTCGACCCCGGTGA
GCATCAAGCAATATCCGATGTCCCAAAAGGCGCGCTTAGGTATTAAGCCGCACA
TTCAGCGTCTGCTGGATCAAGGTATTCTGGTTCCGTGTCAGAGCCCGTGGAATAC
CCCGCTTCTCCCGGTGAAGAAACCGGGCACGAACGATTACCGTCCAGTCCAAGA
CTTGCGCGAAGTTAACAAGCGCGTTGAAGATATTCACCCGACCGTCCCGAACCCG
TACAATCTGCTGAGCGGTCCGCCGCCAAGCCACCAATGGTACACCGTGCTGGATC
TGAAAGATGCTTTCTTCTGTCTGCGTCTGCACCCAACCAGCCAGCCTCTGTTTGCA
TTTGAGTGGCGTGACCCTGAGATGGGTATTAGCGGCCAGCTGACGTGGACCCGCC
TGCCGCAAGGTTTTAAGAATTCCCCTACGCTGTTTAACGAAGCGCTGCACCGTGA
CCTGGCGGATTTCCGTATCCAGCACCCGGACCTGATCTTGCTGCAGTACGTTGAT
GACCTGTTGCTGGCGGCGACGAGCGAGCTGGATTGCCAACAGGGCACCCGTGCG
CTGTTGCAGACCTTGGGTAACCTGGGTTATCGCGCTAGCGCGAAGAAAGCGCAG
ATTTGCCAAAAACAAGTTAAGTATCTGGGCTACCTGTTAAAGGAAGGCCAACGTT
GGCTGACCGAAGCCCGCAAAGAAACTGTCATGGGTCAGCCGACCCCGAAAACGC
CACGCCAACTGCGTAGGTTCTTGGGCAAAGCGGGTTTCTGCCGCCTGTTCATCCC
GGGCTTTGCCGAAATGGCAGCCCCGCTGTATCCGTTGACCAAGCCGGGCACCCTG
TTCAACTGGGGTCCGGACCAGCAGAAAGCGTACCAAGAAATTAAACAAGCACTG
CTGACGGCACCGGCGCTGGGTCTGCCGGACCTGACCAAGCCGTTTGAGCTGTTCG
TGGATGAGAAGCAAGGTTACGCGAAGGGCGTGTTGACCCAGAAATTGGGTCCGT
GGCGTCGTCCGGTTGCATACCTGTCCAAGAAACTGGACCCGGTTGCTGCTGGTTG
GCCGCCTTGCCTGCGCATGGTTGCCGCTATCGCGGTGCTGACTAAAGACGCGGGT
AAGCTGACGATGGGTCAACCGCTGGTGATCGGCGCACCGCATGCAGTCGAGGCC
CTTGTTAAGCAACCGGCAGGAAGATGGCTGAGCAAGGCGCGTATGACGCATTAC
CAGGCACTGCTGTTGGACACCGATCGTGTGCAGTTTGGCCCGGTCGTTGCGCTCA
ACCCGGCGACCCTGCTGCCGCTCCCGGAAGAAGGCTTGCAGCACAACTGTTTGG
ACATCCTGGCAGAGGCGCACGGCACTCGCCCGGATCTGACGGACCAGCCGCTGC
CGGACGCCGATCATACCTGGTATACGAATGGTAGCAGCCTGTTGCAAGAGGGTC
AGCGTAAGGCCGGTGCCGCGGTCACCACCGAGACTGAAGTGATTTGGGCTAAAG
CATTGCCTGCGGGTACCAGCGCGCAGCGTGCCGAGCTGATCGCACTGACCCAAG
CGCTGAAAATGGCTGAGGGTAAGAAACTGAATGTGTACACGGATAGCCGTTATG
CCTTTGCGACCGCCCACATTCACGGCGAGATCTATCGCCGTCGCGGCTGGCTGAC
GTCCAAAGGCAAAGAGATCAAGAATAAAGACGAAATTCTGGCGCTGCTGAAAGC
GCTGTTCCTGCCGAAACGTCTGTCGATCATCCATTGCCCGGGTCACCAGAAAGGC
CACAGCGCAGAGGCGCGTGGTAATCGCATGGCTGACCAGGCTGCGCGTAAAGCC
GCAATTACCGAAACCCCGGACACCAGCACGCTGCTGATCGAGAATAGCAGCCCG
AACAGCCGTCTGATCAAT
(MMLV variant))
SEQ ID NO: 24
TWLSDFPQAWAETGGMGLAVRQAPLIIPLKATSTPVSIKQYPMSQKARLGIKPHIQRL
LDQGILVPCQSPWNTPLLPVKKPGTNDYRPVQDLREVNKRVEDIHPTVPNPYNLLSG
PPPSHQWYTVLDLKDAFFCLRLHPTSQPLFAFEWRDPEMGISGQLTWTRLPQGFKNS
PTLFNEALHRDLADFRIQHPDLILLQYVDDLLLAATSELDCQQGTRALLQTLGNLGY
RASAKKAQICQKQVKYLGYLLKEGQRWLTEARKETVMGQPTPKTPRQLRRFLGKA
GFCRLFIPGFAEMAAPLYPLTKPGTLFNWGPDQQKAYQEIKQALLTAPALGLPDLTK
PFELFVDEKQGYAKGVLTQKLGPWRRPVAYLSKKLDPVAAGWPPCLRMVAAIAVL
TKDAGKLTMGQPLVIGAPHAVEALVKQPAGRWLSKARMTHYQALLLDTDRVQFGP
VVALNPATLLPLPEEGLQHNCLDILAEAHGTRPDLTDQPLPDADHTWYTNGSSLLQE
GQRKAGAAVTTETEVIWAKALPAGTSAQRAELIALTQALKMAEGKKLNVYTDSRY
AFATAHIHGEIYRRRGWLTSKGKEIKNKDEILALLKALFLPKRLSIIHCPGHQKGHSAE
ARGNRMADQAARKAAITETPDTSTLLIENSSPNSRLIN
RTx_(Tgo-RTX)
SEQ ID NO: 25
MVLDTDYITEDGKPVIRIFKKENGEFKIDYDRNFEPYLYALLKDDSAIEDVKKITAER
HGTTVRVVRAEKVKKKFLGRPIEVWKLYFTHPQDVPAIMDKIKEHPAVVDIYEYDIP
FAIRYLIDKGLIPMEGDEELKLLAFDIETLYHEGEEFAEGPILMISYADEEGARVITWK
NIDLPYVDVVSTEKEMIKRFLKVVKEKDPDVLITYNGDNEDFAYLKKRSEKLGVKFI
LGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDV
SRSSTGNLVEWFLLRKAYERNELAPNKPDERELARRHESHAGGYIKEPERGLWENIV
YLDFRSLYPSIIITHNVSPDTLNREGCEEYDVAPQVGHKFCKDFPGFIPSLLGDLLEER
QKVKKRMKATIDPIEKKLLDYRQRAIKILANSLYGYYGYAKARWYCKECAESVIAW
GRQYLETTIREIEEKFGFKVLYADTDGFFATIPGADAETVKKKAKEFLDYINAKLPGL
LELEYEGFYKRGLFVTKKKYAVIDEEDKITTRGLEIVRRDWSEIAKETQARVLEAILK
HGDVEEAVRIVKEVTEKLSKYEVPPEKLVIYKQITRDLKDYKATGPHVAVAKRLAA
RGIKIRPGTVISYIVLKGSGRIVDRAIPFDEFDPAKHKYDAEYYIEKQVLPAVERILRAF
GYRKEDLRYQKTRQVGLGARLKPKT
(Pfu-RTX))
SEQ ID NO: 26
MILDVDYITEEGKPVIRLFKKENGKFKIEHDRTFRPYLYALLRDDSKIE
EVKKITGERHGKIVRIVDVEKVEKKFLGKPITVWKLYLEHPQDVPTIMEKVREHPAV
VDIFEYDIPFAIRYLIDKGLIPMEGEEELKLLAFDIETLYHEGEEFGKGPIIMISYADENE
AKVITWKNIDLPYVEVVSSEREMIKRFLRIIREKDPDIIVTYNGDSFDFPYLAKRAEKL
GIKLTIGRDGSEPKMQRIGDMTAVEVKGRIHFDLYHVITRTINLPTYTLEAVYEAIFGK
PKEKVYADEIAKAWESGENLERVAKYSMEDAKATYELGKEFLPMEIQLSRLVGQPL
WDVSRSSTGNLVEWFLLRKAYERNEVAPNKPSEEEYQRRLHESHTGGFIKEPEKGL
WENIVYLDFRALYPSIIITHNVSPDTLNLEGCKNYDIAPQVGHKFCKDIPGFIPSLLGHL
LEERQKIKTRMKETQDPIEKILLDYRQKAIKLLANSLYGYYGYAKARWYCKECAESV
IAWGRKYLELVWKELEEKFGFKVLYIDTDGLYATIPGGESEEIKKKALEFVKYINSKL
PGLLELEYEGFYKRGLFVTKKRYAVIDEEGKVITRGLEIVRRDWSEIAKETQARVLET
ILKHGDVEEAVRIVKEVIQKLANYEIPPEKLAIYKQITRPLHEYKAIGPHVAVAKKLA
AKGVKIKPGMVIGYIVLRGDGPIVNRAILAEEYDPKKHKYDAEYYIEKQVLPAVLRIL
EGFGYRKEDLRYQKTRQVGLTSRLNIKKS
(Pfu-RTX (exo⁻)
SEQ ID NO: 27
MILDVDYITEEGKPVIRLFKKENGKFKIEHDRTFRPYLYALLRDDSKIEEVKKITGERH
GKIVRIVDVEKVEKKFLGKPITVWKLYLEHPQDVPTIMEKVREHPAVVDIFEYDIPFAI
RYLIDKGLIPMEGEEELKLLAFAIATLYHEGEEFGKGPIIMISYADENEAKVITWKNID
LPYVEVVSSEREMIKRFLRIIREKDPDIIVTYNGDSFDFPYLAKRAEKLGIKLTIGRDGS
EPKMQRIGDMTAVEVKGRIHFDLYHVITRTINLPTYTLEAVYEAIFGKPKEKVYADEI
AKAWESGENLERVAKYSMEDAKATYELGKEFLPMEIQLSRLVGQPLWDVSRSSTGN
LVEWFLLRKAYERNEVAPNKPSEEEYQRRLHESHTGGFIKEPEKGLWENIVYLDFRA
LYPSIIITHNVSPDTLNLEGCKNYDIAPQVGHKFCKDIPGFIPSLLGHLLEERQKIKTRM
KETQDPIEKILLDYRQKAIKLLANSLYGYYGYAKARWYCKECAESVIAWGRKYLEL
VWKELEEKFGFKVLYIDTDGLYATIPGGESEEIKKKALEFVKYINSKLPGLLELEYEG
FYKRGLFVTKKRYAVIDEEGKVITRGLEIVRRDWSEIAKETQARVLETILKHGDVEEA
VRIVKEVIQKLANYEIPPEKLAIYKQITRPLHEYKAIGPHVAVAKKLAAKGVKIKPGM
VIGYIVLRGDGPIVNRAILAEEYDPKKHKYDAEYYIEKQVLPAVLRILEGFGYRKEDL
RYQKTRQVGLTSRLNIKKS
Targ-RTX
SEQ ID NO: 28
MILAADYITKDGKPIVRIFKKENGEFKIELDPHFRPYLYALLRDDSAIEEIMQIKGERH
GKTVRIVDAIKVKKKFLRRPVEVWKLIFEHPQDVPAMMGKIRSHPAVVDIYEYDIPF
AIRYLIDKGLVPMEGEEDLKLLAFDIETFYHEGDEFGKGEIIMISYADDEEAGVITWK
RINLPYVHVVSNEREMIKRFVQIIKEKDPDVIITYNGDNFDLPYLIKRAEKLGVRLLLG
RDKEHPEPKIQRMGDSFAVEIKGRIHFDLFPVVRRTVNLPTYTLEAVYETVLGKQKT
KLGAEEIAAIWETEEGMKKLAQYSMEDAKATYELGREFFPMEAELAKVIGQSVWDV
SRSSTGNLVEWYMLRVAYERNELAPNKPSDEEYKRRLHTTHIGGYIKEPERGLWGNI
VYLDFRSLYPSIIVTHNVSPDTLEREGCQDYEVAPIVGYRFCKDFSGFIPSILENLIETR
QEVKKRMKSTTDPVERKMLDYRQRALKILANSLYGYQGYPKARWYSKECAESVIA
WGRHYLEMSIREIEEKFGFKVLYADTDGFYATIPGEKPDNIKKKAKEFLDYINSKLPG
LLELEYEGFYLRGLFVTKKRYAVIDEDGRITTRGLEVVRRDWSEIAKETQAKVLEAIL
REGSVEKAVEIVKSVVERIAKYKVPLEKLVIHKQITRELKDYKAIGPHVAIAKRLAAK
GIKVKPGTIISYIVLKGGGKIVDRVVLLTEYDPRKHKYDPDYYIDKQVLPAVLRILEAF
GYKKEDLRYQRSKQTGLEARLRR
Targ-RTX (exo⁻)
SEQ ID NO: 29
MILAADYITKDGKPIVRIFKKENGEFKIELDPHFRPYLYALLRDDSAIEEIMQIKGERH
GKTVRIVDAIKVKKKFLRRPVEVWKLIFEHPQDVPAMMGKIRSHPAVVDIYEYDIPF
AIRYLIDKGLVPMEGEEDLKLLAFAIATFYHEGDEFGKGEIIMISYADDEEAGVITWK
RINLPYVHVVSNEREMIKRFVQIIKEKDPDVIITYNGDNFDLPYLIKRAEKLGVRLLLG
RDKEHPEPKIQRMGDSFAVEIKGRIHFDLFPVVRRTVNLPTYTLEAVYETVLGKQKT
KLGAEEIAAIWETEEGMKKLAQYSMEDAKATYELGREFFPMEAELAKVIGQSVWDV
SRSSTGNLVEWYMLRVAYERNELAPNKPSDEEYKRRLHTTHIGGYIKEPERGLWGNI
VYLDFRSLYPSIIVTHNVSPDTLEREGCQDYEVAPIVGYRFCKDFSGFIPSILENLIETR
QEVKKRMKSTTDPVERKMLDYRQRALKILANSLYGYQGYPKARWYSKECAESVIA
WGRHYLEMSIREIEEKFGFKVLYADTDGFYATIPGEKPDNIKKKAKEFLDYINSKLPG
LLELEYEGFYLRGLFVTKKRYAVIDEDGRITTRGLEVVRRDWSEIAKETQAKVLEAIL
REGSVEKAVEIVKSVVERIAKYKVPLEKLVIHKQITRELKDYKAIGPHVAIAKRLAAK
GIKVKPGTIISYIVLKGGGKIVDRVVLLTEYDPRKHKYDPDYYIDKQVLPAVLRILEAF
GYKKEDLRYQRSKQTGLEARLRR
KOD-RTXKOD-RTX
SEQ ID NO: 30
MILDTDYITEDGKPVIRIFKKENGEFKIEYDRTFEPYLYALLKDDSAIEEVKKITAERH
GTVVTVKRVEKVQKKFLGRPVEVWKLYFTHPQDVPAIMDKIREHPAVIDIYEYDIPF
AIRYLIDKGLVPMEGDEELKLLAFDIETLYHEGEEFAEGPILMISYADEEGARVITWK
NVDLPYVDVVSTEREMIKRFLRVVKEKDPDVLITYNGDNFDFAYLKKRCEKLGINFA
LGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVS
RSSTGNLVEWFLLRKAYERNELAPNKPDEKELARRHQSHEGGYIKEPERGLWENIVY
LDFRSLYPSIIITHNVSPDTLNREGCKEYDVAPQVGHRFCKDFPGFIPSLLGDLLEERQ
KIKKRMKATIDPIERKLLDYRQRAIKILANSLYGYYGYARARWYCKECAESVIAWGR
EYLTMTIKEIEEKYGFKVIYSDTDGFFATIPGADAETVKKKAMEFLKYINAKLPGALE
LEYEGFYKRGLFVTKKKYAVIDEEGKITTRGLEIVRRDWSEIAKETQARVLEALLKD
GDVEKAVRIVKEVTEKLSKYEVPPEKLVIHKQITRDLKDYKATGPHVAVAKRLAAR
GVKIRPGTVISYIVLKGSGRIVDRAIPFDEFDPTKHKYDAEYYIEKQVLPAVERILRAF
GYRKEDLRYQKTRQVGLSARLKPKGT
(WT Targ); DNA-directed DNA polymerase [Thermococcus
argininiproducens]; NCBI Reference Sequence: WP_251948234.1
SEQ ID NO: 31
MILAADYITKDGKPIVRIFKKENGEFKIELDPHFRPYIYALLRDDSAIEEIMQIKGERHG
KTVRIVDAIKVKKKFLRRPVEVWKLIFEHPQDVPAMRGKIRSHPAVVDIYEYDIPFAK
RYLIDKGLVPMEGEEDLKLLAFDIETFYHEGDEFGKGEIIMISYADDEEAGVITWKRI
NLPYVHVVSNEREMIKRFVQIIKEKDPDVIITYNGDNFDLPYLIKRAEKLGVRLLLGR
DKEHPEPKIQRMGDSFAVEIKGRIHFDLFPVVRRTVNLPTYTLEAVYETVLGKQKTK
LGAEEIAAIWETEEGMKKLAQYSMEDAKATYELGREFFPMEAELAKVIGQSVWDVS
RSSTGNLVEWYMLRVAYERNELAPNKPSDEEYKRRLRTTYIGGYVKEPERGLWGNI
VYLDFRSLYPSIIVTHNVSPDTLEREGCQDYEVAPIVGYRFCKDFSGFIPSILENLIETR
QEVKKRMKSTTDPVERKMLDYRQRALKILANSYYGYQGYPKARWYSKECAESVTA
WGRHYIEMSIREIEEKFGFKVLYADTDGFYATIPGEKPDNIKKKAKEFLDYINSKLPG
LLELEYEGFYLRGFFVTKKRYAVIDEDGRITTRGLEVVRRDWSEIAKETQAKVLEAIL
REGSVEKAVEIVKSVVERIAKYKVPLEKLVIHEQITRELKDYKAIGPHVAIAKRLAAK
GIKVKPGTIISYIVLKGGGKISDRVVLLTEYDPRKHKYDPDYYIDNQVLPAVLRILEAF
GYKKEDLRYQRSKQTGLEAWLRR

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Engineered Family B Polymerases with Reverse Transcriptase Activity

This example demonstrates the generation of engineered nucleic acid processing enzymes (e.g., engineered recombinant Family-B polymerases, engineered enzymes; engineered DNA polymerase enzymes; engineered polymerases) having reverse transcriptase activity and substantially lacking or completely lacking strand displacement amplification activity. Wild type DNA polymerase enzymes that can be engineered using the method disclosed herein can include, but are not limited to, Pyrococcus furiosus (pfu) polymerase (SEQ ID NO: 1), Thermococcus gorgonarius polymerase (Tgo polymerase) (SEQ ID NO: 10), Thermococcus litoralis (VENT®) polymerase (SEQ ID NO: 20), Pyrococcus sp. (Deep Vent)polymerase (SEQ ID NO: 21), Thermococcus sp. (9°N) polymerase (SEQ ID NO: 22), or Thermococcus argininiproducens (Targ) polymerase (SEQ ID NO: 31). The sequences of exemplary engineered family B polymerases are shown in FIGS. 6A-D.

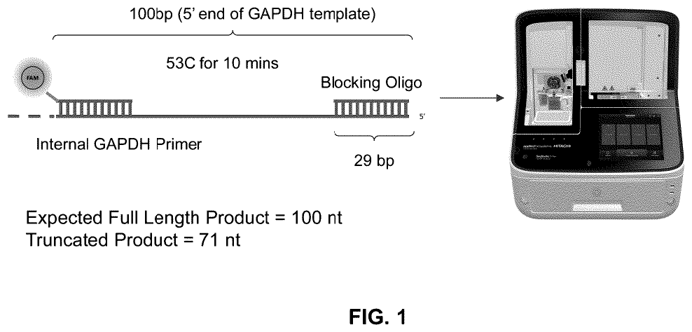

To determine whether the engineered family B polymerases disclosed herein could in fact exhibit reverse transcriptase activity with minimal to no strand displacement activity, an assay disclosed in FIG. 1 was developed. FIG. 1 shows an overview of the assay used to test the engineered family B polymerases of the present disclosure using 100 bp from the 5′ end of Glyceraldehyde 3-phosphate dehydrogenase (GAPDH) as a template and a blocking oligo of about 29 nucleotides was used. The full length product was expected to be 100 nucleotides (nt), and a truncated product was expected to be 71 nt in the presence of a blocking oligo. The four enzymes tested included a reverse transcriptase enzyme, a variant MMLV RT (control; SEQ ID NO: 23 and SEQ ID NO: 24); an engineered Thermococcus kodakarensis (KOD-RTX; Family B Engineered Polymerase); an engineered Thermococcus gorgonarius (Tgo-RTX), which was engineered to have mutations on a Tgo backbone that resulted in a Tgo variant enzyme (SEQ ID NO: 11, 12, 25) with minimal strand displacement activity; and Bst 3.0 (a Family A Engineered Polymerase) with strong strand displacement activity and no exonuclease (exo) activity. Bst 3.0 DNA Polymerase is an in silico designed homologue of Bacillus stearothermophilus DNA Polymerase I, Large Fragment. Bst 3.0 is a fusion protein comprising a polymerase domain fused to a novel nucleic acid binding domain for improved isothermal amplification performance and increased reverse transcription activity.

### TgoRTx has a Minimal Strand Displacement Activity

FIGS. 2A-B show chromatographs illustrating amplification products obtained using the control MMLV RT with RT reagent B in the absence (FIG. 2A) or the presence (FIG. 2B) of a blocking oligo. The absence of a pick at the expected start of the blocking oligo (about 71 nt) demonstrated that the control MMLV RT enzyme completely displaced the 29 bp blocking oligo without any issues. The size standard for the CE assay uses a Liz dye. The DNA was monitored with a FAM dye (e.g., the primer was FAM labeled). Since the dyes are different, there was a slight discrepancy between the size reported by the instrument (based off the Liz size standards) and DNA size. As such, the obtained sizing on CE was off by ˜5 nt. However, the difference was corrected for in the data shown in all the figures. For example, in FIG. 2, the sizing has been corrected as not to be off. RT reagent B is the RT buffer used on commercially available 10× Genomics single cell product. See e.g., 10×genomics.com/support/single-cell-gene-expression/documentation/steps/library-prep/chromium-next-gem-single-cell-3-reagent-kits-safety-data-sheets-v-3-1-chemistry; or Chromium Next GEM Single Cell 3′ GEM Kit v3.1.

FIGS. 3A-B show chromatographs illustrating amplification products obtained using an engineered Tgo RT enzyme (Tgo RTX; SEQ ID NO: 11, 12, or 25) with RT reagent B in the absence (FIG. 3A) or the presence (FIG. 3B) of the 29 bp blocking oligo. FIG. 3B shows some peaks at the expected start of the blocking oligo, demonstrating that the Tgo-RTx had minimal strand displacement activity. In particular, Tgo-RTx displaced about 6 nt of the blocking oligo before termination. FIG. 3B shows that Tgo-RTx could not produce a full length product in the presence of a blocking oligo. For example, Tgo-RTX generated an extension product of a size indicating maximum displacement of about 6 nucleotides before termination. The expected start of the blocking oligo was about 70 nt, while the major peak appeared around 76 nucleotides (FIG. 3B). In contrast, there was no peak after the expected start of the blocking oligo when KOD-RT was used (FIG. 4B).

FIGS. 4A-B show chromatographs illustrating amplification products obtained using an engineered KOD RT enzyme (KOD RTX; SEQ ID NO: 7, 9, or 30) with RT reagent B in the absence (FIG. 4A) or the presence (FIG. 4B) of the 29 bp blocking oligo. FIG. 4 demonstrates that the KOD-RTX has no strand displacement activity and its reverse transcriptase activity is not as efficient as Tgo-RTX. This could be due to difficulty transcribing through secondary structure and the blocking oligo may alleviate this. This effect may be resolved by performing the amplification assay at higher temperature.

As shown in FIG. 4B, the majority of termination occurs 1-2 bp upstream of the 5′ end of the blocking oligo. It is possible that longer incubation times and/or higher [RT] could have helped push the amplification reaction to completion (e.g., to obtain a full-length product). FIG. 4B also shows that KOD RTX did not seem to displace any base of the blocking oligo.

These results also show that Tgo-RTX was able to produce a full-length product without any intermediates (FIG. 3A vs. FIG. 4A) when compared to KOD-RTX in the absence of a blocking oligo. However, Tgo-RTX had some minimal strand displacement activity when compared to KOD-RTX (FIG. 3B vs. FIG. 4B).

FIGS. 5A-B show chromatographs illustrating amplification products obtained using Bst 3.0 with RT reagent B in the absence (FIG. 5A) or the presence (FIG. 5B) of the 29 bp blocking oligo. FIGS. 5A-B demonstrate that Bst 3.0, as expected, strand displaced very well. However, Bst 3.0 it was not as good as the control MMLV enzyme shown in FIGS. 2A-B because some truncated products were generated as shown in FIG. 5A.

Thus, this example demonstrates for the first time that KOD-RTX, a Family B Engineered Polymerase showed no strand displacement activity; while Tgo-RTX (KOD-RTX mutations on Tgo backbone) showed possible minimal strand displacement activity. Bst 3.0 (Family A Engineered Polymerase) showed strong strand displacement activity but had exonuclease activity. Last, the control MMLV RT enzyme showed the strongest strand displacement activity.

### Example 2: Rationale Design of Engineered Polymerase with Reverse Transcriptase Activity

Engineered polymerase enzymes that were capable of reverse transcribing RNA at temperature ranging from 37° C. to 70° C. were engineered by rational design using a Thermococcus gorgonarius (Tgo) polymerase (SEQ ID NO: 10), a Pyrococcus furiosus (pfu) polymerase (SEQ ID NO: 1), a VENT® polymerase (SEQ ID NO: 20), a Deep Vent polymerase (SEQ ID NO: 21), a 9°N polymerase (SEQ ID NO: 22), or a Targ polymerase (SEQ ID NO: 31). This rational design identified a group of 20 amino acids that were important for generating the reverse transcriptase activity: I2V, I38L, R97M, K118I, M137L, R381H, Y384H, V389L, K466R, F493L, T514I, I521L, F587L, E664K, G711V, N735K, and W768R in SEQ ID NO: 1 or SEQ ID NO: 10.

The novel engineered thermophilic enzymes functioned as a DNA polymerase and was capable of amplifying DNA. The dual RT/DNA polymerase activity was demonstrated by showing that the engineered thermophilic enzymes amplified DNA products following PCR amplification of a sample comprising only an RNA template. Furthermore, the engineered thermophilic enzymes reverse transcribed an RNA and generated an amplification product at low (53° C.) and high (68° C.) temperatures.

In contrast, a control Moloney Murine Leukemia Virus (MMLV) reverse-transcriptase (MMLV RT) variant reverse transcribed that same RNA at low temperatures (53° C.) but failed to reverse transcribe that RNA at high temperature (68° C.). The engineered thermophilic polymerase enzymes disclosed also herein demonstrated greater efficiency at reverse transcribing long RNA molecules (1300 nt) at temperatures ranging from 53° C. to 68° C. as compared to the control MMLV variant RT enzyme. In fact, the relative amount of product generated using the MMLV RT enzyme was about half (approximately 600) when compared to the TgoRTx product generation (approximately 1200). In addition, the TgoRTx product generation was increased at 68° C. when compared to a product generated at 53° C.

TgoRT and TgoRTx were More Efficient than a MMLV RT Variant Enzyme for RNA Analysis of Droplets of Less than 1 nL.

A clear body of evidence demonstrated that reverse transcription of mRNA from a single cell was inhibited from an unknown component(s) present in a cell lysate when the reaction volume was less than about 1 nL. To overcome this inhibition and facilitate the utilization of smaller reaction volumes, the control MMLV RT variant enzyme was tested in droplets containing picoliter-sized reaction volumes. The control MMLV RT enzyme variant effectively reduced the previously identified inhibition of reverse transcription in a 350 pL reaction volume in comparison to a second available mutant MMLV RT enzyme. However, the observation that TgoRT and TgoRTx were more efficient at high temperatures than either MMLV RT enzymes attested to the novelty and unexpected effect of the engineered family B polymerases in single cell analysis of RNA in small volume.

In addition to the thermophilic Tgo enzyme that is exonuclease proficient (TgoRT; SEQ ID NO: 12), a thermophilic Tgo enzyme that was exonuclease deficient (TgoRTx; SEQ ID NO: 11 or 25) was engineered.

TgoRT and TgoRTx were More Efficient than Corresponding T. kodakarensis Enzymes

The engineered thermophilic T. gorgonarius reverse transcriptase was found during experimentation to be more efficient at reverse transcribing a template than engineered reverse transcriptases known in the art. For example, a DNA polymerase from Thermococcus kodakarensis (KOD polymerase; SEQ ID NO: 6 or 8) was engineered to reverse transcribe RNA. See e.g., Elefson et al Science 336(6079): 341-344 (2016). This reverse transcriptase was engineered from the backbone of KOD DNA polymerase generated cDNA from RNA substrates using regular amplification techniques. When tested in a high throughput system, such as spatial array transcriptomics assay, single cell transcriptomics assay, a single cell profiling reaction, or related single cell sequencing system, the efficiency of the engineered KOD polymerase (KODRTx) was less than that seen from the TgoRTx disclosed herein. The KODRTx enzyme was also unable to reverse transcribe an RNA template at 53° C. However, the engineered TgoRTx of the present disclosure showed robust activity at 53° C. Indeed, the reverse transcriptase efficiency of the engineered TgoRTx at 53° C. was equal to or perhaps more efficient at transcribing a 1300 nt template compared to a variant Moloney Murine Leukemia Virus (MMLV) reverse-transcriptase (MMLV RT) enzyme.

A sequence comparison showed that wild type T. gorgonarius DNA polymerase (Tgo) is about 92.63% identical to wild type T. kodakarensis polymerase (KodPol) (FIGS. 6A-D and 7). While not being bound to any particular theory, it is possible that T. gorgonarius DNA polymerase is a better enzyme for high throughput amplification assays such as the single cell analysis or cellular RNA analysis using droplets in emulsion. In addition, T. gorgonarius DNA polymerase may be more efficiency for RNA analysis in a volume (e.g., droplet) that is less than 1 nL.

Additional Engineered Polymerases with Reverse Transcriptase Activity

For RTL-based gap fill, e.g., without limitation RNA targeted ligation SNP detection, a polymerase is needed that can fill in any gaps between adjacent RTL probes without displacing the probe down-stream. WT reverse transcriptases have varying levels of stand displacement activity (e.g., control enzyme in FIGS. 2A-B), making them unsuitable for gap fill RTL. As such the present inventors proposed using a B-family DNA polymerase that has been engineered to be a reverse transcriptase. As noted above, an engineered Tgo enzyme was generated and showed minimal strand displacement activity.

Accordingly, the present inventors contemplated using Pfu polymerase, which is another homologous B-family polymerase with ˜79% sequence identity to KOD. Pfu is used commercially in Gibson cloning for gap fill, meaning it lacks any appreciable strand displacement activity. Thus, an RT version of Pfu polymerase considered to be a perfect or ideal candidate for RTL gap fill.

Similar to the rationale design used with Tgo above, the present inventors engineered Pyrococcus furiosus (pfu) polymerase (SEQ ID NO: 1), VENT® polymerase (SEQ ID NO: 20), Deep Vent polymerase (SEQ ID NO: 21), 9°N polymerase (SEQ ID NO: 22), and Targ polymerase (SEQ ID NO: 31), to have a reverse transcriptase that is capable of reverse transcribing RNA at temperature ranging from 37° C. to 70° C. and while substantially lacking strand displacement amplification activity, or while not having detectable strand displacement activity. This rationale design identified a group of 20 amino acids that were important for generating the reverse transcriptase activity: 2V, 38L, 97M, 118I, 137L, 381H, 384H, V389I, 466R, 493L, 514I, 521L, 587L, 664K, 711V, 735K, and 768R of the KOD polymerase. Such engineered family B polymerases are disclosed in SEQ ID NO: 2-5, and 26-30. An alignment of these sequences is shown in FIGS. 6A-D.

It is expected that enzymes comprising an amino acid sequence selected from the group consisting of SEQ ID NO: 2-5, and 26-30 will have reverse transcriptase activity and substantially will lack strand displacement amplification activity, will not have detectable strand displacement activity.

### Example 3: Engineered Tgo Polymerases Showed Enhanced Gap Filling at High Temperatures

Given the surprising non-strand displacing property of Tgo-RTX and Tgo-RTXo in producing full length products suitable for gap filling reactions in Example 1 and FIGS. 3A-B using a 100 nt gap fill assay, the Tgo-RTX (exo⁺) and RTXo (exo⁻) were further assayed using a 200 nt gap fill assay. Four different concentrations of each of Tgo RTX and Tgo RTXo were tested: 0.125 μM, 0.250 μM, 0.500 μM, and 1.00 μM at four different temperatures: 37° C., 42° C., 48° C., and 53° C.

FIGS. 8A-B show an overview of the assay used to further demonstrate that Tgo-RTX and Tgo-RTXo had minimal strand displacing activity and these engineered family B polymerases were therefore suitable for gap filling reactions. FIG. 8A shows the 259 bp 5′ end of Glyceraldehyde 3-phosphate dehydrogenase (GAPDH) that was used as a template; the 30-bp FAM-labeled primer (probe) that can be extended by Tgo-RTX or Tgo-RTXo; and a 29 bp 5′ Phosho blocking oligonucleotide (blocking oligo) that can be displaced by an enzyme having a strand-displacement activity.

The expected size of the full length product was 259 nucleotides (nt) in the absence of the blocking oligo and about 230 nt in the presence of the blocking oligo (see also FIG. 1). The products were grouped into six categories for quantification. Group 1, “Fully Displaced” meant a full length product with full strand displacement and had the expected size of 259 nt. Group 2: “Truncated products” were products with less than 230 nt (i.e., the amplification reaction terminated before the expected start of the blocking oligo). Group 3: “Gapfill” referred to optimal desired products that had exactly 230 nt (i.e., no strand displacement). Group 4: “Partial Displaced” products referred to products that had between 231 nt to 258 nt (i.e., limited strand displacement). “Partial Displaced” products were also desirable products. Group 5: “Truncated Probe” referred to products that were shorter than the FAM-primer (fewer than 30 nt) and indicated issues with the probe during the assay. Group 6: “Probe” referred to products that were exactly the length of the FAM-primer (30 nt in length). These indicated issues with the amplification reaction itself.

FIG. 8B shows an exemplary chromatograph obtained from the assay illustrated in FIG. 8A and shows a single amplification product that illustrated obtaining of a gapfill target product (230 nt) and a product with limited strand displacement (e.g., size 231 nt) or “Partial Displaced” product.

FIGS. 9A-B show bar graphs quantifying amplification products obtained using various concentrations (0.125 μM, 0.250 μM, 0.500 μM, and 1.00 μM) of Tgo-RTX(exo⁻) (FIG. 9A) and Tgo-RT(exo⁺) (FIG. 9B) at a temperature of 37° C. A significant fraction of the products generated from the reactions were Truncated products (i.e., Products with less than 230 nt). This suggested that amplification reactions were terminated before the expected start of the blocking oligo. Thus, a significant amount of incomplete extension for both variants was observed. Such truncated products are not desired for a gapfilling reaction.

In Tgo-RTX(exo⁻) (FIG. 9A) and Tgo-RT(exo⁺) (FIG. 9B), the percentage of the “Partial Displaced” products increased with the concentration of the enzyme. However, the percent of “Partial Displaced” products was higher in reactions comprising the Tgo-RTX(exo⁻) and the percentage of Gapfill was higher in reactions comprising the Tgo-RTX(exo⁺). For example, about 35% of products were Gapfill when 1.000 μM Tgo-RTX(exo⁺) was used; and about 40% of products were Partial Displaced products when 1.000 μM Tgo-RTX(exo⁻) was used.

At a temperature of 42° C., reactions comprising Tgo-RTX(exo⁻) (FIG. 10A) generated products that were mostly “Partial Displaced” products (231 nt to 258 nt). 45% to about 60% of the products were Partial Displaced products. In reactions comprising Tgo-RTX(exo⁺) (FIG. 10B) 50-55% of the products were “Truncated Products” (less than 230 nt) and 25% to about 45% were Gapfill (230 nt). For all conditions at this temperature, at least 30% of products were truncated.

At a temperature of 48° C., reactions comprising Tgo-RTX(exo⁻) (FIG. 11A) generated products that were mostly “Partial Displaced” products (231 nt to 258 nt). 97% (0.125 μM), 95% (0.250 μM), 90% (0.500 μM), and 45% (1.000 μM) were “Partial Displaced” products. 20% (1.000 μM) were Fully Displaced products. In reactions comprising Tgo-RTX(exo⁺) (FIG. 11B) most products were Gapfill (230 nt). 55% (0.125 μM), 60% (0.250 μM), 40% (0.500 μM), and 30% (1.000 μM) were Gapfill products; and 15% (0.125 μM), 5% (0.250 μM), 30% (0.500 μM), and 47% (1.000 μM) were Truncated Products. In particular, and surprisingly, over half of products were the gapfill optimal target products using TgoRTX (exo+) at concentrations of 0.125 μM and 0.250 PM.

At a temperature of 53° C., reactions comprising Tgo-RTX(exo⁻) (FIG. 12A) generated products were mostly “Partial Displaced” products (231 nt to 258 nt) at lower enzyme concentrations (0.125 μM and 0.250 μM) and “Fully displaced” at higher enzyme concentrations (0.500 μM and 1.00 μM). 99% (0.125 μM), 90% (0.250 μM), 50% (0.500 μM), and 0% (1.000 μM) were Partial Displaced products and 1% (0.125 μM), 10% (0.250 μM), 50% (0.500 μM), and 90% (1.000 μM) were Fully Displaced products.

In reactions comprising Tgo-RTX(exo⁺) (FIG. 12B) most products were Gapfill (230 nt). 65% (0.125 μM), 68% (0.250 μM), 55% (0.500 μM), and 45% (1.000 μM) were Gapfill products and 30% (0.125 μM), 30% (0.250 μM), 25% (0.500 μM), and 10% (1.000 μM) were Partial Displaced products. Exo− variant began to fully displace the blocking oligo at 0.250 μM. Lower concentrations of exo⁺ gave nearly complete conversion to desired product (gapfill or partial displace). Surprisingly, over 50% of products were gapfill optimal products for the exo+ version at three of the concentrations tested (0.125 M, 0.250 μM, and 0.500 μM).

Unexpectedly, the engineered Tgo polymerase provided desired products useful for gapfilling at a variety of concentrations of the enzyme and temperatures of the reaction (particularly 48° C. and 53° C. (FIGS. 8A-B, 9A-B, 10A-B, 11A-B, and 12A-B). In particular, Tgo-RTX (exo⁺) generated mostly “Gapfill” products (i.e., product length 230 nt) 48° C. (FIG. 11B); and 53° C. (FIG. 12B).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

1. A method of producing a polymerized nucleic acid product, the method comprising:
(a) contacting an engineered family B polymerase with a probe-hybridized nucleic acid template and deoxyribonucleotide triphosphates, wherein the probe-hybridized nucleic acid template comprises a first probe end hybridized to a first region and a second probe end hybridized to a second region, and an unhybridized region between the first region and the second region; and
(b) generating an extended product by extending the first probe end in the unhybridized region;
wherein the engineered family B polymerase comprises mutations to positions 38, 97, 118, 137, 381, 384, 389, 466, 493, 514, 521, 587, 664, 711, 735, and 768 corresponding to positions of SEO ID NO: 10.

2. The method of claim 1, wherein the probe-hybridized nucleic acid template comprises an RNA template.

3. The method of claim 2, wherein:
(a) the first probe end and the second probe end are of a same probe molecule; or
(b) the first probe end and the second probe end are of different probe molecules.

4. The method of claim 1, wherein steps (a) and (b) are performed in a biological sample.

5. The method of claim 4, wherein the biological sample comprises a cell or tissue sample.

6. The method of claim 5, wherein the cell or tissue sample comprises a Formalin-Fixed Paraffin-Embedded (FFPE) sample, a formalin-fixed sample, a paraffin-embedded sample, a frozen sample, or a fresh sample.

7. The method of claim 1, wherein the unhybridized region comprises a site of genetic variability.

8. The method of claim 1, wherein the method further comprises ligating a 3′ end of the product to a 5′ end of the second probe end.

9. The method of claim 1, wherein the method further comprises modifying the extension product or an amplification copy thereof, to incorporate a barcode.

10. The method of claim 9, wherein the barcode comprises a spatial barcode.

11. The method of claim 10, wherein the method is performed in a spatial location in the biological sample, and the spatial barcode identifies the spatial location.

12. The method of claim 9, wherein the barcode comprises a single cell barcode.

13. The method of claim 12, wherein modifying the extension product is performed in a partitioned cell.

14. (canceled)

15. The method of claim 1, wherein the mutations comprise: 38L, 97M, 118I, 137L, 381H, 384H, 389I, 466R, 493L, 514I, 521L, 587L, 664K, 711V, 735K, and 768R corresponding to positions of SEQ ID NO: 10.

16. The method of claim 1, wherein the engineered family B polymerase is selected from the group consisting of Pyrococcus furiosus (pfu) polymerase, Thermococcus gorgonarius polymerase (Tgo polymerase), a Thermococcus kodakarensis (KOD1) polymerase, a Thermococcus litoralis (VENT®) polymerase, a Pyrococcus sp. (Deep Vent) polymerase, a Thermococcus sp. (9°N) polymerase, or a Thermococcus argininiproducens (Targ) polymerase.

17. The method of claim 1, wherein the engineered family B polymerase has an amino acid sequence selected from the group consisting of: SEQ ID NO: 2, SEQ ID NO: 3, SEQ ID NO: 4, SEQ ID NO: 5, SEQ ID NO: 11, SEQ ID NO: 12, SEQ ID NO: 25, SEQ ID NO: 26, SEQ ID NO: 27, SEQ ID NO: 28, SEQ ID NO: 29, and SEQ ID NO: 30.

18.-20. (canceled)

21. The method of claim 1, wherein the engineered family B polymerase further comprises one or more mutations that reduce or abolish exonuclease activity.

22. The method of claim 21, wherein the one or more mutations that reduce or abolish exonuclease activity are at one or more of positions 2, 93, 141, 143, and 485, with respect to the positions of SEQ ID NO: 10.

23. The method of claim 22, wherein the one or more mutations that reduce or abolish exonuclease activity comprise mutations at positions 141 and 143 with respect to the positions of SEQ ID NO: 10.

24.-64. (canceled)
